(19) United States
(12) Patent Application Publication
(10) Pub. No.: US 2026/0256751 A1
(43) Pub. Date: Sep. 3, 2026

(54) COMBINATION OF BETA-2-ADRENERGIC RECEPTOR AGONISTS AND GLP-1 RECEPTOR AGONISTS FOR USE IN TREATING HYPERGLYCAEMIA

(71) Applicant: ATROGI AB, Solna (SE)

(72) Inventors: Tore Bengtsson, Vaxholm (SE); Benjamin Pelcman, Stockholm (SE); Anastasia Kalinovich, Solna (SE)

(21) Appl. No.: 19/162,093

(22) PCT Filed: Mar. 6, 2024
(86) PCT No.: PCT/EP2024/055881

§ 371 Date: Sep. 4, 2025

(30) Foreign Application Priority Data
Mar. 6, 2023 (GB) 2303229.5

(51) Int. Cl. A61K 31/44; A61K 38/26; A61K 38/28; A61P 3/10; A61P 5/50

(52) U.S. Cl. CPC A61K 31/44; A61K 38/26; A61K 38/28; A61P 3/10; A61P 5/50

(57) ABSTRACT

There is provided herein a β₂-adrenergic receptor agonist, or a pharmaceutically acceptable salt thereof, for use in the treatment and/or prophylaxis of hyperglycaemia or a disorder characterized by hyperglycaemia, wherein the treatment and/or prophylaxis further comprises administration of: (a) insulin or a therapeutic insulin derivative; or (b) a therapeutic agent stimulating the release of insulin.

### FIELD OF THE INVENTION

The present invention relates to methods for the treatment and/or prophylaxis of hyperglycaemia or a disorder characterized by hyperglycaemia. In particular, the invention relates to methods for the treatment and/or prophylaxis of hyperglycaemia or a disorder characterized by hyperglycaemia, involving treatment and/or prophylaxis with a combination of a β₂-adrenergic receptor agonist and insulin or a therapeutic insulin derivative, or a therapeutic agent stimulating the release of insulin, and to compositions and kits-of-parts for use in such methods.

### BACKGROUND OF THE INVENTION

The listing or discussion of an apparently prior-published document in this specification should not necessarily be taken as an acknowledgement that the document is part of the state of the art or is common general knowledge.

Hyperglycaemia, or high blood sugar, is a condition in which an excessive amount of glucose circulates in the blood plasma. If not treated, hyperglycaemia can be a serious problem, potentially developing into life-threatening conditions such as ketoacidosis. For example, chronic hyperglycemia may cause injury to the heart, and is strongly associated with heart attacks and death in subjects with no coronary heart disease or history of heart failure. There are various causes of hyperglycaemia, including diabetes and severe insulin resistance.

Severe insulin resistance (SIR) is a condition wherein the patient experiences very low levels of (or, in extreme cases, no significant) response to insulin. There are several syndromes characterized by SIR, including Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndrome, pseudoacromegaly, and lipodystrophy. The majority of these conditions have genetic causes, such as mutations in the insulin receptor gene. The prevalence for Donohue's syndrome, Rabson-Mendenhall syndrome and Type A syndrome of insulin resistance, has been reported to vary from about 50 reported cases to 1 in 100,000. However, since some diseases are severe and extremely rare, it is likely that many patients do not get diagnosed before they die, particularly in less developed areas of the world. Thus, the exact number of patients with these syndromes is difficult to assess.

The current standard for hyperglycaemia treatment in patients having SIR is a controlled diet, supplemented with drugs affecting insulin receptor sensitivity, such as metformin, or insulin supplement. However, particularly for disorders caused by mutations in the insulin receptor gene, this treatment is not sufficiently effective and ultimately proves unsuccessful.

Diabetes comprises two distinct diseases, type 1 (or insulin-dependent diabetes) and type 2 (insulin-independent diabetes), both of which involve the malfunction of glucose homeostasis. Type 2 diabetes affects more than 400 million people in the world and the number is rising rapidly. Complications of type 2 diabetes include severe cardiovascular problems, kidney failure, peripheral neuropathy, blindness and, in the later stages of the disease, even loss of limbs and, ultimately death. Type 2 diabetes is characterized by insulin resistance in skeletal muscle and adipose tissue, and there is presently no definitive cure. Most treatments used today are focused on remedying dysfunctional insulin signalling or inhibiting glucose output from the liver but many of those treatments have several drawbacks and side effects. There is thus a great interest in identifying novel insulin-independent ways to treat type 2 diabetes.

In type 2 diabetes, the insulin-signalling pathway is blunted in peripheral tissues such as adipose tissue and skeletal muscle. Methods for treating type 2 diabetes typically include lifestyle changes, as well as insulin injections or oral medications to regulate glucose homeostasis. People with type 2 diabetes in the later stages of the disease develop ‘beta-cell failure’ i.e. the inability of the pancreas to release insulin in response to high blood glucose levels. In the later stages of the disease patients often require insulin injections in combination with oral medications to manage their diabetes. Further, most common drugs have side effects including downregulation or desensitization of the insulin pathway and/or the promotion of lipid incorporation in adipose tissue, liver and skeletal muscle. There is thus a great interest in identifying novel ways to treat metabolic diseases including type 2 diabetes that do not include these side effects.

Following a meal, increased blood glucose levels stimulate insulin release from the pancreas. Insulin mediates normalization of the blood glucose levels. Important effects of insulin on glucose metabolism include facilitation of glucose uptake into skeletal muscle and adipocytes, and an increase of glycogen storage in the liver. Skeletal muscle and adipocytes are responsible for insulin-mediated glucose uptake and utilization in the fed state, making them very important sites for glucose metabolism.

The signalling pathway downstream from the insulin receptor has been difficult to understand in detail. In brief, control of glucose uptake by insulin involves activation of the insulin receptor (IR), the insulin receptor substrate (IRS), the phosphoinositide 3-kinase (PI3K) and thus stimulation of phosphatidylinositol (3,4,5)-triphosphate (PIP3), the mammalian target of rapamycin (also called the mechanistic target of rapamycin, mTOR), Akt/PKB (Akt) and TBC1D4 (AS160), leading to translocation of the glucose transporter 4 (GLUT4) to the plasma membrane. Akt activation is considered necessary for GLUT4 translocation.

It should be noted that skeletal muscles constitute a major part of the body weight of mammals and have a vital role in the regulation of systemic glucose metabolism, being responsible for up to 85% of whole-body glucose disposal. Glucose uptake in skeletal muscles is regulated by several intra- and extracellular signals. Insulin is the most well studied mediator but others also exist. For example, AMP activated kinase (AMPK) functions as an energy sensor in the cell, which can increase glucose uptake and fatty acid oxidation. Due to the great influence skeletal muscles have on glucose homeostasis it is plausible that additional mechanisms exist. In the light of the increased prevalence of type 2 diabetes, it is of great interest to find and characterize novel insulin-independent mechanisms to increase glucose uptake in muscle cells.

Blood glucose levels may be regulated by both insulin and catecholamines, but they are released in the body in response to different stimuli. Whereas insulin is released in response to the rise in blood sugar levels (e.g. after a meal), epinephrine and norepinephrine are released in response to various internal and external stimuli, such as exercise, emotions and stress, and also for maintaining tissue homeostasis. Insulin is an anabolic hormone that stimulates many processes involved in growth including glucose uptake, glycogen and triglyceride formation, whereas catecholamines are mainly catabolic.

Although insulin and catecholamines normally have opposing effects, it has been shown that they have similar actions on glucose uptake in skeletal muscle (Nevzorova et al., Br. J. Pharmacol, 137, 9, (2002)). In particular, it has been reported that catecholamines stimulate glucose uptake via adrenergic receptors (Nevzorova et al., Br. J. Pharmacol, 147, 446, (2006); Hutchinson, Bengtsson Endocrinology 146, 901, (2005)) to supply muscle cells with an energy-rich substrate. Thus it is likely that in mammals, including humans, the adrenergic and the insulin systems can work independently to regulate the energy needs of skeletal muscle in different situations. Since insulin also stimulates many anabolic processes, including some that promote undesired effects such as stimulation of lipid incorporation into tissues, leading to e.g. obesity, it would be beneficial to be able to stimulate glucose uptake by other means; for example, by stimulation of the adrenergic receptors (ARs).

All ARs are G protein-coupled receptors (GPCRs) located in the cell membrane and characterized by an extracellular N-terminus, followed by seven transmembrane a-helices (TM-1 to TM-7) connected by three intracellular (IL-1 to IL-3) and three extracellular loops (EL-1 to EL-3), and finally an intracellular C-terminus. There are three different classes of ARs, with distinct expression patterns and pharmacological profiles: α₁-, α₂- and β-ARs. The α₁-ARs comprise the α_{1A}, α_{1B} and α_{1D} subtypes while α₂-ARs are divided into α_{2A}, α_{2B} and α_{2C}. The β-ARs are also divided into the subtypes β₁, β₂, and β₃, of which β₂-AR is the major isoform in skeletal muscle cells. ARs are G protein coupled receptors (GPCRs) that signal through classical secondary messengers such as cyclic adenosine monophosphate (cAMP) and phospholipase C (PLC).

Many effects occurring downstream of ARs in skeletal muscles have been attributed to classical secondary messenger signalling, such as increase in cAMP levels, PLC activity and calcium levels. Stimulation involving the classical secondary messengers has many effects in different tissues. For example, it increases heart rate, blood flow, airflow in lungs and release of glucose from the liver, which all can be detrimental or be considered unwanted side effects if stimulation of ARs should be considered as a type 2 diabetes treatment. Adverse effects of classical AR agonists are, for example, tachycardia, palpitation, tremor, sweats, agitation and increased glucose levels in the blood (glucose output from the liver). It would thus be beneficial to be able to activate ARs without activating these classical secondary messengers, such as cAMP, to increase glucose uptake in peripheral tissues without stimulating the unwanted side effects.

Glucose uptake is mainly stimulated via facilitative glucose transporters (GLUT) that mediate glucose uptake into most cells. GLUTs are transporter proteins that mediate transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied isoforms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in, for example, the brain, kidney, and liver. GLUT4 is the major isoform involved in insulin stimulated glucose uptake. The mechanism whereby insulin signalling increases glucose uptake is mainly via GLUT4 translocation from intracellular storage to the plasma membrane. It is known that GLUT4 translocation is induced by stimulation of the β₂-adrenergic receptor.

There are currently several treatments for diabetes and related conditions that act by increasing insulin sensitivity. These may be used in treatment alone, thus increasing sensitivity to naturally occurring insulin production, or in combination with therapeutic agents that act by increasing insulin levels, which may allow such agents to be used in lower doses and/or less frequently.

However, such insulin sensitizing treatments are known to have a range of limitations, including an association with serious adverse events.

Thus, there remains a need for safe and effective insulin sensitizing agents for use in the treatment of diabetes and related conditions.

### DESCRIPTION OF THE INVENTION

We have now surprisingly found that activation of the β₂-adrenergic receptor results in increased sensitivity of the insulin receptor, such that treatment and/or prophylaxis with β₂-adrenergic receptor agonists in combination with insulin or insulin releasing agents allows for effective treatment and/or prophylaxis of conditions characterized by high blood sugar levels (i.e. hyperglycaemia), such as diabetes (e.g. type 2 diabetes).

### Uses in Medicine

In a first aspect of the invention there is provided a β₂-adrenergic receptor agonist, or a pharmaceutically acceptable salt thereof, for use in the treatment and/or prophylaxis of hyperglycaemia or a disorder characterized by hyperglycaemia, wherein the treatment and/or prophylaxis further comprises administration of:
(a) insulin or a therapeutic insulin derivative; or
(b) a therapeutic agent stimulating the release of insulin.

Unless indicated otherwise, all technical and scientific terms used herein have the same meaning as commonly understood by one of ordinary skill in the art to which this invention pertains.

Preferences and options for a given aspect, embodiment, feature or parameter of the invention should, unless the context indicates otherwise, be regarded as having been disclosed in combination with any and all preferences and options for all other aspects, features and parameters of the invention.

Wherever the word “about” is employed herein (for example, in the context of doses of active ingredients) it will be appreciated that such variables are approximate and as such may vary by ±10%, for example ±5% and preferably ±2% (e.g. ±1%) from the numbers specified herein.

Wherever the word “optionally” is employed in relation to features described herein it will take its normal meaning, namely that the relevant feature may or may not be present.

In an alternative first aspect of the invention there is provided the use of a β₂-adrenergic receptor agonist, or a pharmaceutically acceptable salt thereof, in the manufacture of a medicament for the treatment and/or prophylaxis of hyperglycaemia or a disorder characterized by hyperglycaemia, wherein the treatment and/or prophylaxis further comprises administration of:
(a) insulin or a therapeutic insulin derivative; or
(b) a therapeutic agent stimulating the release of insulin.

In an alternative first aspect of the invention there is provided the use of a β₂-adrenergic receptor agonist, or a pharmaceutically acceptable salt thereof, in the treatment and/or prophylaxis of hyperglycaemia or a disorder characterized by hyperglycaemia, wherein the treatment and/or prophylaxis further comprises administration of:
(a) insulin or a therapeutic insulin derivative; or
(b) a therapeutic agent stimulating the release of insulin.

In an alternative first aspect of the invention there is provided a compound that is:
(a) insulin or a therapeutic insulin derivative; or
(b) a therapeutic agent stimulating the release of insulin
for use in the treatment and/or prophylaxis of hyperglycaemia or a disorder characterized by hyperglycaemia, wherein the use further comprises administration of a β₂-adrenergic receptor agonist, or a pharmaceutically acceptable salt thereof.

In an alternative first aspect of the invention there is provided the use of a compound that is:
(a) insulin or a therapeutic insulin derivative; or
(b) a therapeutic agent stimulating the release of insulin
in the manufacture of a medicament for the treatment and/or prophylaxis of hyperglycaemia or a disorder characterized by hyperglycaemia, wherein the use further comprises administration of a β₂-adrenergic receptor agonist, or a pharmaceutically acceptable salt thereof.

In an alternative first aspect of the invention there is provided the use of a compound that is:
(a) insulin or a therapeutic insulin derivative; or
(b) a therapeutic agent stimulating the release of insulin
in the treatment and/or prophylaxis of hyperglycaemia or a disorder characterized by hyperglycaemia, wherein the use further comprises administration of a β₂-adrenergic receptor agonist, or a pharmaceutically acceptable salt thereof.

In an alternative first aspect of the invention there is provided a combination of a β₂-adrenergic receptor agonist, or a pharmaceutically acceptable salt thereof, and a compound that is:
(a) insulin or a therapeutic insulin derivative; or
(b) a therapeutic agent stimulating the release of insulin
for use in the treatment and/or prophylaxis of hyperglycaemia or a disorder characterized by hyperglycaemia.

In an alternative first aspect of the invention there is provided a method for the treatment and/or prophylaxis of hyperglycaemia or a disorder characterized by hyperglycaemia comprising:
(i) administration of a therapeutically effective amount of
(a) a therapeutically effective amount of insulin or a therapeutic insulin derivative; or
(b) a therapeutically effective amount of a therapeutic agent stimulating the release of insulin, and
(ii) administration of a β₂-adrenergic receptor agonist, or a pharmaceutically acceptable salt thereof,
to a patient in need thereof.

In an alternative first aspect of the invention there is provided a method for the treatment and/or prophylaxis of hyperglycaemia or a disorder characterized by hyperglycaemia comprising administration of a therapeutically effective amount of
(a) insulin or a therapeutic insulin derivative; or
(b) a therapeutic agent stimulating the release of insulin
to a patient in need thereof, wherein the patient is also being treated with a therapeutically effective amount of a β₂-adrenergic receptor agonist, or a pharmaceutically acceptable salt thereof.

In an alternative first aspect of the invention there is provided a method for the treatment and/or prophylaxis of hyperglycaemia or a disorder characterized by hyperglycaemia comprising administration of a therapeutically effective amount of a β₂-adrenergic receptor agonist, or a pharmaceutically acceptable salt thereof, to a patient in need thereof, wherein the patient is also being treated with a therapeutically effective amount of
(a) insulin or a therapeutic insulin derivative; or
(b) a therapeutic agent stimulating the release of insulin.

For the avoidance of doubt, uses, methods, compositions and kits-of-parts of other aspects of the invention as described herein (including all embodiments thereof) may have any of the particular features described above for the first aspect of the invention, including all combinations thereof.

For the avoidance of doubt, treatments (and associated prophylaxis) as described herein may be referred to herein as the “treatments of the invention”, or the like.

The skilled person will understand that references to the “treatment of” a particular condition (and similarly “treating”) take their normal meanings in the field of medicine. In particular, the terms may refer to achieving a reduction in the severity of one or more clinical symptom associated with the condition. In particular, the terms may refer to achieving a reduction in the severity of one or more clinical symptom associated with the condition.

As used herein, references to patients will refer to a living subject being treated, including mammalian (e.g. human) patients. In particular embodiments of the relevant aspects of the invention (e.g. the first aspect of the invention), the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

As used herein, the term prophylaxis includes references to the prevention of (and, similarly, preventing) the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In particular embodiments, references to use in and methods for the treatment or prophylaxis of diseases and disorders as specified herein will refer in particular to uses in and methods for treatment of such diseases and disorders.

In a particular embodiment, the treatment is of a disorder (which may also be referred to as a condition or disease) characterised by hyperglycaemia.

In particular embodiments of the first aspect of the invention, the disorder is type 2 diabetes, such as type 2 diabetes of a sub-type selected from the list consisting of maturity-onset diabetes in the young (MODY), ketosis-prone diabetes in adults, latent autoimmune diabetes of adults (LADA), and gestational diabetes.

In further embodiments, the disorder is type 1 diabetes, particularly wherein the treatment further comprises treatment with insulin (or a derivative and/or functional mimetic thereof).

In particular embodiments, compounds of the invention (i.e. compounds of formula I, including all embodiments thereof) are for use in the treatment of type 2 diabetes (or useful in the manufacture of a medicament for such treatment, or useful in a method for such treatment, as described herein).

In further particular embodiments, the treatment of type 2 diabetes is in a non-obese patient.

For the avoidance of doubt, the skilled person will understand that patients with a Body Mass Index (BMI) of greater than 30 are considered to be obese.

In particular embodiments, the treatment may be of hyperglycaemia in a patient who is at risk of developing type 2 diabetes, which condition may be defined as pre-diabetes. Thus, compounds of the invention may be useful in the prevention of type 2 diabetes (e.g. in a patient having pre-diabetes).

As used herein, the term prevention (and, similarly, preventing) includes references to the prophylaxis of the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In more particular embodiments, the type 2 diabetes is characterised by the patient displaying severe insulin resistance (SIR).

In further embodiments, the treatment may be of hyperglycaemia in a patient having type 1 diabetes. Thus, compounds of the invention may be useful in the treatment of hyperglycaemia in type 1 diabetes.

In particular embodiments, the disorder characterised by hyperglycaemia is Type 1 diabetes or Type 2 diabetes.

The skilled person will understand that compounds of the invention may be useful in treating hyperglycaemia in patients having impaired insulin production, such as in patients having cystic fibrosis. Thus, in further embodiments, the disorder characterized by hyperglycaemia is cystic fibrosis-related diabetes.

In particular embodiments that may be mentioned, the disorder characterised by hyperglycaemia is (or is characterized by) severe insulin resistance (SIR), which may be understood by those in the art to refer to disorders wherein typically the subject has normal, or in some cases increased, insulin production but significantly reduced insulin sensitivity. In particular instances, such patients may be non-obese (e.g. being of a healthy weight). Thus, in particular embodiments, such treatments are performed in patients who are not defined as being obese (e.g. in patients who are defined as being of a healthy weight).

For example, SIR may be identified in a patient based in said patient having fasting insulin >150 pmol/L and/or a peak insulin on glucose tolerance testing of >1,500 pmol/L, particularly in individuals with a BMI <30 kg/m² (which patient may otherwise have normal glucose tolerance).

More particularly, SIR may be characterised by the patient having no significant response to the presence of insulin, which may result from a defect (e.g. a genetic defect) in the function of the insulin receptor.

Particular disorders that may be characterised by SIR include: Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

More particular disorders that may be characterised by SIR include Donohue's syndrome and Type A syndrome of insulin resistance and, yet more particularly, Rabson-Mendenhall syndrome.

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of type 2 diabetes, such as treatment with one or more other therapeutic agent that is useful in the treatment of type 2 diabetes as known to those skilled in the art, such as therapies comprising requiring the patient to undergo a change of diet and/or undertake exercise regiments, and/or surgical procedures designed to promote weight loss (such as gastric band surgery).

The skilled person will understand that treatments of the first aspect of the invention may further comprise (i.e. be combined with) further (i.e. additional/other) treatment(s) for the same condition.

### Compounds of the Invention

As described herein, the present invention requires the use of a β₂-adrenergic receptor agonist, examples of which will be known to those skilled in the art.

The skilled person will understand that compounds referred to herein, such as compounds referred to as a β₂-adrenergic receptor agonist, may be provided in the form of a pharmaceutically acceptable salt.

Pharmaceutically acceptable salts include acid addition salts and base addition salts, each of which may be in the form of salts in varying ratios of compound to counter ion (e.g. including hemi salts). Such salts may be formed by conventional means, for example by reaction of a free acid or a free base form of a compound comprised in the formulations of the invention with one or more equivalents of an appropriate acid or base, optionally in a solvent, or in a medium in which the salt is insoluble, followed by removal of said solvent, or said medium, using standard techniques (e.g. by rotary evaporation under reduced pressure, by freeze-drying or by filtration). Salts may also be prepared by exchanging a counter-ion of a compound comprised in the formulations of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, hemi-tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, 1-naphtoate, 2-naphtoate, 1-hydroxy-2-naphtoate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1,2-ethanedisulphonate, 1- or 2- naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds as described herein may exist as solids, and thus the scope of the invention includes all amorphous, crystalline and part crystalline forms thereof, and may also exist as oils. Where such compounds exist in crystalline and part crystalline forms, such forms may include solvates, which are included in the scope of the invention. The compounds may also exist in solution.

The skilled person will understand that references to an agonist will refer to compounds suitable for acting as such when administrated to a subject to be treated (i.e. a patient, e.g. a human, in need thereof). Suitable compounds may include compounds which provide the required effect and compounds which are converted to compounds providing the required effect after administration (i.e. in vivo), which compounds may be referred to as pro-drugs. Particular compounds that may be mentioned are compounds which elicit the required effect.

For the avoidance of doubt, the term “agonist” may be understood to indicate an agent (i.e. a compound) that induces activation of the relevant receptor to produce a biological response (e.g. in a subject, such as a human), such as by binding to the relevant receptor. As such, the term may also refer to partial agonists (which will be understood to refer to compounds that activate a given receptor, but have only partial efficacy at the receptor relative to a full agonist).

Agonists (and partial agonists) may display, for example, half maximal effective concentration (EC₅₀) values of less than about 100 μM, such as less than about 10 μM, or less than about 1 μM (e.g. less than about 200, about 150 or about 120 nM).

Unless otherwise stated or clear from the context, references herein to agonists will also include pharmaceutically-acceptable (e.g. “protected”) derivatives of compounds which may not possess the relevant activity per se, but may be administered (e.g. parenterally or orally) to a patient and thereafter be metabolised in the body to form compounds possessing the required activity, which compounds may be referred to as prodrugs. Suitable prodrugs of compounds as described herein will be known to those skilled in the art, such as suitable esters (e.g. methyl or ethyl esters, and the like).

For the avoidance of doubt, unless otherwise stated or clear from the context, references to compounds that are agonists, and pharmaceutically acceptable salts thereof, will include compounds that are prodrugs of such agonists, and pharmaceutically acceptable salts thereof.

Suitable β₂-adrenergic receptor agonists (which may also be referred to as β₂-agonists) may include those known to those skilled in the art.

In certain embodiments, suitable β₂-adrenergic receptor agonists will include those that are selective, which term will be known to those skilled in the art (i.e. compounds that are agonists of the relevant receptor(s) but which do not cause significant activation of other P-adrenergic receptors).

Suitable β₂-adrenergic receptor agonists can be identified using techniques known to those skilled in the art, including those as described in the examples provided herein.

Suitable β₂-adrenergic receptor agonists that may be employed in the various aspects of the invention include, but are not limited to, those described in: WO 2004/071388, EP 0 272 976, FR 2647310, DE 2 157 040, DE 2212600, DE 2015573, ZA 6705591, DE 2128258, WO 91/09596, GB 1 199 630, DE 4209989, BE 611502, NL 7804582, EP 0 043 807, WO 2008/022038, DE 2413102, U.S. Pat. No. 2,308,232, BE 823841, BE 660244, WO 2000/075114, WO 2005/102350, WO 2005/110990, JP 56055355, AT 285583, U.S. Pat. Nos. 4,223,137, 3,056,836, FR 1324914, DE 638650, DD 45721, U.S. Pat. No. 3,801,631, DE 2259282, DE 2300614, EP 0 290 122, US 2004/0266867, US 2010/0022658, US 2010/0022659, DE 2157040, GB 2133986, WO 2006/122788, Woo et al., Molecular Pharmacology, (2009), 75(1) 158-165, Baur et al., J. Med. Chem., (2010), 53(9), 3675-3684, Kaiser et al., J. Med. Chem. (1974), 17(1) 49-57, Baker et al., J. Pharmacology and Experimental Therapeutics, (2006), 319(1), 439-446, Engelhardt et al., Arzneimittel-Forschung, (1972), 22(5), 869-76, WO 2019/241744, WO 2019/241736, WO 2020/198466, WO 2021/003161, WO 2021/081292, WO 2021/127210 and WO 2021/247934, the relevant disclosures of each of which (e.g. the examples compounds described therein, and pharmaceutically acceptable salts thereof, and associated methods of preparation) are hereby incorporated by reference in their entirety.

For the avoidance of doubt, references to patent publications will typically refer to the initial publication of the full patent specification with the relevant publication number (which may be indicated by the suffix “Al”).

Further suitable β₂-adrenergic receptor agonists that may be employed in the various aspects of the invention (which compounds may be identified as also being suitable β₂-adrenergic receptor agonists) include those described in the following publications, the contents of which are hereby incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2017/153737
WO 2019/053429
WO 2019/053426
WO 2019/053425
WO 2019/053427
WO 2020/188299
WO 2020/188301
WO 2022/063895
WO 2022/063889
WO 2023/046885
WO 2023/046882
WO 2023/105035
WO 2023/203223
WO 2020/198466

A particular β₂-adrenergic receptor agonist that may be mentioned is the following compound:

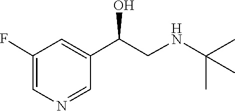

and pharmaceutically acceptable salts thereof.

Thus, a particular β₂-adrenergic receptor agonist that may be mentioned is (R)-2-(tert-butylamino)-1-(5-fluoropyridin-3-yl)ethan-1-ol, and pharmaceutically acceptable salts thereof.

Particular pharmaceutically acceptable salts of the above-mentioned compound (i.e. (R)-2-(tert-butylamino)-1-(5-fluoropyridin-3-yl)ethan-1-ol) that may be mentioned include the hemi-tartrate and dihydrochloride salts (such as the hemi-tartrate salt).

A further particular β₂-adrenergic receptor agonist that may be mentioned is the following compound:

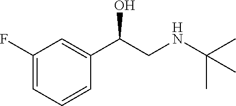

and pharmaceutically acceptable salts thereof.

Thus, a particular β₂-adrenergic receptor agonist that may be mentioned is (R)-2-(tert-butylamino)-1-(3-fluorophenyl)ethan-1-ol, and pharmaceutically acceptable salts thereof.

Particular pharmaceutically acceptable salts of (R)-2-(tert-butylamino)-1-(3-fluorophenyl)ethan-1-ol that may be mentioned include the HCl (hydrochloride) salt.

In particular embodiments, references to a specific stereoisomer of a compound may refer to the specific stereoisomer being present (e.g. in a composition or formulation comprising the same) in the substantial absence of the corresponding opposite stereoisomer.

As used herein, references to the substantial absence of the corresponding opposite stereoisomer may refer to the desired stereoisomer being present at a purity of at least 80% (e.g. at least 90%, such as at least 95%) relative to the opposite stereoisomer. Alternatively, in such instances, compounds may be indicated to be present in the substantial absence of the compound in the other configuration, which may indicate that the compound in the relevant configuration is present in an enantiomeric excess (e.e.) of at least 80% (such as at least 90%, at least 95%, at least 98% or, particularly, at least 99%, for example at least 99.9%).

In some embodiments, the compound in the relevant configuration is present in an enantiomeric excess (e.e.) of at least 90% (such as at least 95%, at least 98% or, particularly, at least 99%, for example at least 99.9%).

In certain embodiments the invention, the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672, trantinterol, clenproperol, clenpenterol, brombuterol, ractopamine and abediterol, and pharmaceutically acceptable salts thereof.

In further certain embodiments, the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672 and trantinterol, and pharmaceutically acceptable salts thereof.

In particular embodiments, the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol, trantinerol and abediterol, and pharmaceutically acceptable salts thereof.

In further particular embodiments, the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol and trantinerol, and pharmaceutically acceptable salts thereof.

In more particular embodiments, the β₂-adrenergic receptor agonist is selected from the group consisting of formoterol, arformoterol, clenbuterol, (R)-clenbuterol, bamethane, (R)-bamethane, and tulobuterol, and pharmaceutically acceptable salts thereof.

In yet more particular embodiments, the β₂-adrenergic receptor agonist is selected from the group consisting of formoterol, arformoterol, clenbuterol, (R)-clenbuterol, bamethane, (R)-bamethane and tulobuterol, and pharmaceutically acceptable salts thereof.

For the avoidance of doubt, the structures of bamethane (CAS: 3703-79-5) and (R)-bamethane (CAS: 912804-58-1) are shown below.

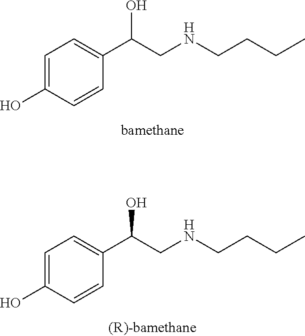

For the avoidance of doubt, in the case of a discrepancy between the name of the compound and the structure drawn in this specification, the structure should prevail.

In further embodiments, the β₂-adrenergic receptor agonist is selected from the group consisting of formeterol, arformeterol, salmeterol, clenbuterol, tulobuterol, bambuterol vilanterol, indacaterol, olodaterol, carmoterol and abediterol, and pharmaceutically acceptable salts thereof.

In yet further embodiments, the β₂-adrenergic receptor agonist is selected from the group consisting of salbutamol, ritodrine, colterol, hexaprenaline, tulobuterol and isoxsuprine, and pharmaceutically acceptable salts thereof.

In particular embodiments, the β₂-adrenergic receptor agonist is clenbuterol or (R)-clenbuterol, or a pharmaceutically acceptable salt thereof.

For the avoidance of doubt, the compound clenbuterol may be understood to have the following structure:

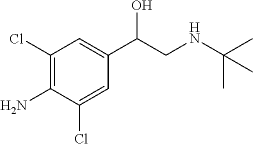 [SMILES: CC(C)(C)NCC(O)c1cc(Cl)c(N)c(Cl)c1]

In particular embodiments, the β₂-adrenergic receptor agonist is tulobuterol or (R)-tulobuterol, or a pharmaceutically acceptable salt thereof.

For the avoidance of doubt, the compound tulobuterol may be understood to have the following structure:

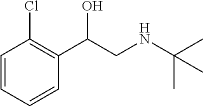

For the avoidance of doubt, the international nonpropriety name (INN) or developmental drug code (e.g. BRL-47672) for a compound generally indicates the stereochemical configuration of the compound, or a particular mixture of stereoisomers (e.g. a racemate). Within the scope of the present invention, where relevant and unless context indicates otherwise (for example where both the racemate and a single stereoisomer are explicitly named), such names may also be considered to encompass separate stereoisomers that display the relevant biological activity, and which have not presently been assigned an alternative INN or developmental drug code.

In particular embodiments, the INN or developmental drug code should be understood to represent the compound to which the relevant name or code has been assigned only.

Where no INN or developmental drug code is available for a compound, the compound may be identified by its Chemical Abstracts Service Registry Number (CAS number). As referred to herein, the indication “CAS: XXXXXX-XX-X” (wherein the number of figures in the first group may vary) is used to identify such compounds. Where relevant and unless context indicates otherwise, the CAS number for a compound may also be considered to encompass other stereoisomers, or mixtures thereof, that display the relevant biological activity, and which have not presently been assigned alternative CAS numbers (as described above for INNs and developmental drug codes).

In particular embodiments, the CAS number should be understood to represent the compound to which the relevant name or code has been assigned only.

In particular embodiments, the β₂-adrenergic receptor agonist is selected from the group consisting of (R)-bamethane, bamethane, clencyclohexerol, radopamine, tulobuterol, and (R)-tulobuterol.

The present invention also embraces pharmaceutical formulations comprising isotopically-labelled compounds, which are identical to the compounds recited herein but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the invention also encompasses pharmaceutical formulations comprising deuterated compounds, i.e. in which one or more hydrogen atoms are replaced by the hydrogen isotope deuterium.

As described herein, it will also be understood that certain compounds acting as β₂-adrenergic receptor agonists are able to activate the β₂-adrenergic receptor without inducing significant cAMP production.

Thus, particular β₂-adrenergic receptor agonists that may be mentioned include those able to activate the β₂-adrenergic receptor without (or with only a minimal effect in) inducing cAMP production.

Further, in particular embodiments, the methods and uses as described herein may be performed without inducing (or without inducing significant levels of) cAMP production.

In particular, the β₂-adrenergic receptor agonist as described herein may be further described as being a β₂-adrenergic receptor agonist that does not induce significant cAMP (i.e. levels and/or production thereof).

The skilled person will be able to determine the level of cAMP production provided by compounds, such as those referred to herein, using techniques known to those skilled in the art, such as those described in the examples as provided herein.

For example, the level of cAMP production induced by a given compound can be determined by reference to the amount induced by a defined concentration of that compound relative to that induced by the same concentration of a reference compound, such as isoprenaline, using techniques known to those skilled in the art (e.g. following the protocol as described in the biological examples provided in WO 2019/053427, i.e. in cells, such as differentiated L6-myotubes, having been stimulated with isoprenaline or the compound with a final concentration of 1×10⁻⁵ M, for 15 min in stimulation buffer, such as HBSS supplemented with 1% BSA, 5 mM HEPES and 1 mM IBMX, at pH 7.4).

In such embodiments, compounds that do not induce significant cAMP may be defined as being compounds that induce less than 50% (or, in some embodiments, less than 25%) of the cAMP induced by isoprenaline (e.g. in accordance with the protocol described above).

Particular compounds acting as β₂-adrenergic receptor agonists which are able to activate the β₂-adrenergic receptor without (or with only a minimal effect in) inducing cAMP production include those described in the following publications, the contents of which are incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2017/153737
WO 2019/053429
WO 2019/053426
WO 2019/053425
WO 2019/053427
WO 2020/188299
WO 2020/188301
WO 2022/063895
WO 2022/063889
WO 2023/046885
WO 2023/046882
WO 2023/105035
WO 2023/203223

Compounds described in publications referenced and incorporated herein were screened in accordance with the procedures outlined in Biological example 1 and Biological example 2 of WO 2019/053427. Test results are given in Table 1. If a compound at 10 μM shows activity of more than 75% of that of isoproterenol at 10 μM, the activity is denoted with +++; if it is between 75 and 50% it is denoted with ++; if it is between 50 and 25% it is denoted with +; if it less than 25% it is denoted with −. Compounds are drawn as salt-free molecules in Table 1, but the tested compounds might have been containing additional salt or solvent components that do not contribute to the biological activity.

In the event that there is a discrepancy between nomenclature and the structure of compounds as depicted graphically, it is the latter that presides (unless contradicted by any experimental details that may be given and/or unless it is clear from the context). Synthetic procedures and further biological data will be found in the relevant publications.

Therefore, particular β₂-adrenergic receptor agonists that may be mentioned are provided in Table 1 below.

TABLE 1
Particular compounds of the invention

| | | | Biological | Biological |
|---|---|---|---|---|
| | | | example 1 | example of |
| | | | of WO | WO |
| | | | 2019/ | 2019/ |
| | | | 053427 | 053427 |
| | Structure | Name | (GU) | (cAMP) |
| 1 | 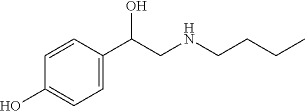 | 2-(butylamino)-1-(p- hydroxyphenyl)-1- ethanol | +++ | − |
| 2 | 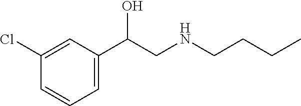 | 2-(butylamino)-1-(m- chlorophenyl)-1-ethanol | + | − |
| 3 | 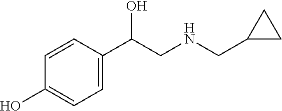 [SMILES: Oc1ccc(C(O)CNCC2CC2)cc1] | p-{2- [(cyclopropylmethyl)ami- no]-1- hydroxyethyl}phenol | ++ | − |
| 4 | 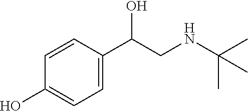 | 2-(tert-butylamino)-1- (p-hydroxyphenyl)-1- ethanol | +++ | + |
| 5 | 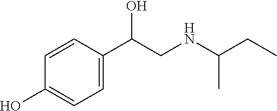 | 2-(sec-butylamino)-1- (p-hydroxyphenyl)-1- ethanol | ++ | − |
| 6 | 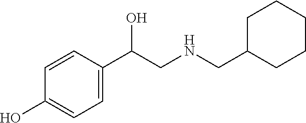 [SMILES: Oc1ccc(C(O)CNCC2CCCCC2)cc1] | 2- [(cyclohexylmethyl)ami- no]-1-(p- hydroxyphenyl)-1- ethanol | +++ | − |
| 7 | 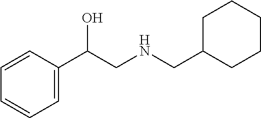 [SMILES: OC(CNCC1CCCCC1)c1ccccc1] | 2- [(cyclohexylmethyl)ami- no]-1-phenyl-1-ethanol | + | − |
| 8 | 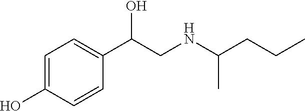 [SMILES: CCCC(C)NCC(O)c1ccc(O)cc1] | 1-(p-hydroxyphenyl)-2- (1-methylbutylamino)- 1-ethanol | ++ | + |
| 9 | 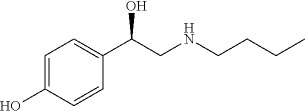 | (R)-2-(butylamino)-1- (p-hydroxyphenyl)-1- ethanol | +++ | − |
| 10 | 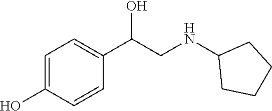 [SMILES: Oc1ccc(C(O)CNC2CCCC2)cc1] | p-[2- (cyclopentylamino)-1- hydroxyethyl]phenol | ++ | − |
| 11 | 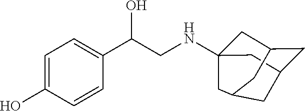 [SMILES: Oc1ccc(C(O)CNC23CC4CC(CC(C4)C2)C3)cc1] | p-[2-(1- adamantanylamino)-1- hydroxyethyl]phenol | + | − |
| 12 | 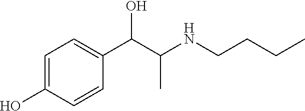 [SMILES: CCCCNC(C)C(O)c1ccc(O)cc1] | 2-(butylamino)-1-(p- hydroxyphenyl)-1- propanol | ++ | − |
| 13 | 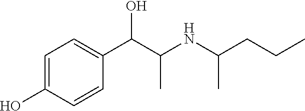 [SMILES: CCCC(C)NC(C)C(O)c1ccc(O)cc1] | 1-(p-hydroxyphenyl)-2- (1-methylbutylamino)- 1-propanol | ++ | − |
| 14 | 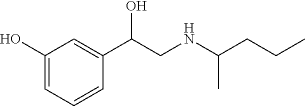 [SMILES: CCCC(C)NCC(O)c1cccc(O)c1] | 1-(m-hydroxyphenyl)-2- (1-methylbutylamino)- 1-ethanol | +++ | − |
| 15 | 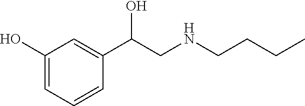 | 2-(butylamino)-1-(m- hydroxyphenyl)-1- ethanol | ++ | − |
| 16 | 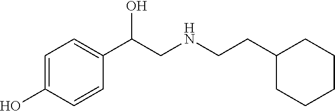 [SMILES: Oc1ccc(C(O)CNCCC2CCCCC2)cc1] | 2-(2- cyclohexylethylamino)- 1-(p-hydroxyphenyl)-1- ethanol | + | − |
| 17 | 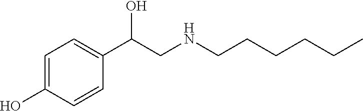 [SMILES: CCCCCCNCC(O)c1ccc(O)cc1] | 2-(hexylamino)-1-(p- hydroxyphenyl)-1- ethanol | + | − |
| 18 | 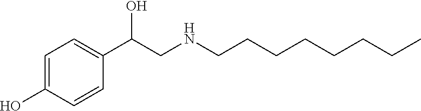 [SMILES: CCCCCCCCNCC(O)c1ccc(O)cc1] | 1-(p-hydroxyphenyl)-2- (octylamino)-1-ethanol | + | − |
| 19 | 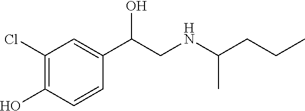 [SMILES: CCCC(C)NCC(O)c1ccc(O)c(Cl)c1] | 1-(3-chloro-4- hydroxyphenyl)-2-(1- methylbutylamino)-1- ethanol | + | − |
| 20 | 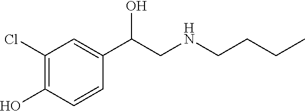 [SMILES: CCCCNCC(O)c1ccc(O)c(Cl)c1] | 2-(butylamino)-1-(3- chloro-4- hydroxyphenyl)-1- ethanol | + | − |
| 21 | 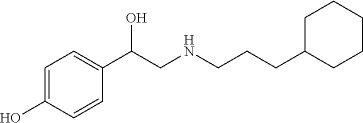 [SMILES: Oc1ccc(C(O)CNCCCC2CCCCC2)cc1] | 2-(3- cyclohexylpropylamino)- 1-(p-hydroxyphenyl)-1- ethanol | +++ | − |
| 22 | 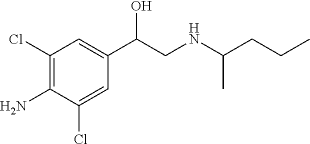 [SMILES: CCCC(C)NCC(O)c1cc(Cl)c(N)c(Cl)c1] | 1-(4-amino-3,5- dichlorophenyl)-2-(1- methylbutylamino)-1- ethanol | +++ | ++ |
| 23 | 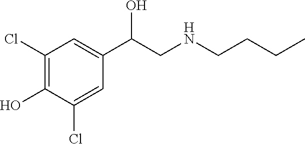 [SMILES: CCCCNCC(O)c1cc(Cl)c(O)c(Cl)c1] | 2-(butylamino)-1-(3,5- dichloro-4- hydroxyphenyl)-1- ethanol | − | − |
| 24 | 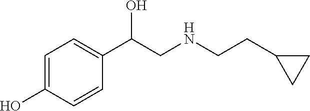 [SMILES: Oc1ccc(C(O)CNCCC2CC2)cc1] | p-[2-(2- cyclopropylethylamino)- 1-hydroxyethyl]phenol | ++ | − |
| 25 | 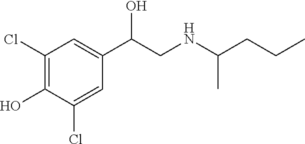 [SMILES: CCCC(C)NCC(O)c1cc(Cl)c(O)c(Cl)c1] | 1-(3,5-dichloro-4- hydroxyphenyl)-2-(1- methylbutylamino)-1- ethanol | − | − |
| 26 | 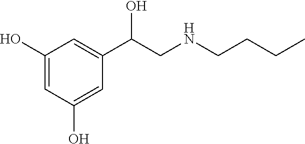 [SMILES: CCCCNCC(O)c1cc(O)cc(O)c1] | 5-[2-(butylamino)-1- hydroxyethyl]resorcinol | ++ | − |
| 27 | 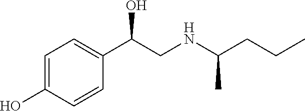 [SMILES: CCC[C@@H](C)NC[C@H](O)c1ccc(O)cc1] | (R)-2-[(R)-1- methylbutylamino]-1- (p-hydroxyphenyl)-1- ethanol | +++ | + |
| 28 | 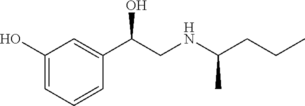 [SMILES: CCC[C@@H](C)NC[C@H](O)c1cccc(O)c1] | (R)-2-[(R)-1- methylbutylamino]-1- (m-hydroxyphenyl)-1- ethanol | +++ | ++ |
| 29 | 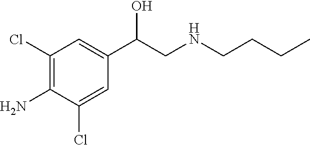 [SMILES: CCCCNCC(O)c1cc(Cl)c(N)c(Cl)c1] | 1-(4-amino-3,5- dichlorophenyl)-2- (butylamino)-1-ethanol | +++ | + |
| 30 | 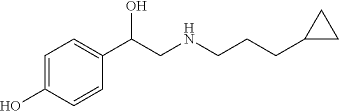 [SMILES: Oc1ccc(C(O)CNCCCC2CC2)cc1] | 2-(3- cyclopropylpropylamino)- 1-(p-hydroxyphenyl)- 1-ethanol | +++ | − |
| 31 | 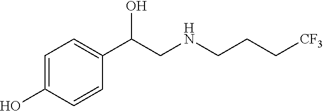 [SMILES: Oc1ccc(C(O)CNCCCC(F)(F)F)cc1] | 1-(p-hydroxyphenyl)-2- (4,4,4- trifluorobutylamino)-1- ethanol | ++ | n/a |
| 32 | 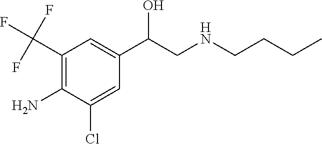 [SMILES: CCCCNCC(O)c1cc(Cl)c(N)c(C(F)(F)F)c1] | 1-[4-amino-3-chloro-5- (trifluoromethyl)phenyl]- 2-(butylamino)-1- ethanol | +++ | + |
| 33 | 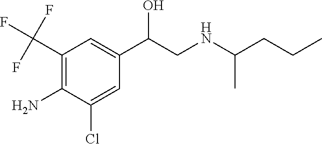 [SMILES: CCCC(C)NCC(O)c1cc(Cl)c(N)c(C(F)(F)F)c1] | 1-[4-amino-3-chloro-5- (trifluoromethyl)phenyl]- 2-(1- methylbutylamino)-1- ethanol | ++ | +++ |
| 34 | 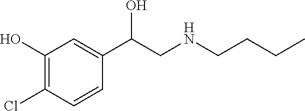 [SMILES: CCCCNCC(O)c1ccc(Cl)c(O)c1] | 2-(butylamino)-1-(4- chloro-3- hydroxyphenyl)-1- ethanol | +++ | − |
| 35 | 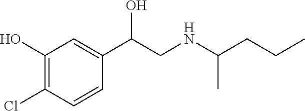 [SMILES: CCCC(C)NCC(O)c1ccc(Cl)c(O)c1] | 1-(4-chloro-3- hydroxyphenyl)-2-(1- methylbutylamino)-1- ethanol | +++ | − |
| 36 | 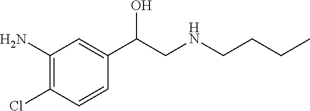 [SMILES: CCCCNCC(O)c1ccc(Cl)c(N)c1] | 1-(3-amino-4- chlorophenyl)-2- (butylamino)-1-ethanol | + | − |
| 37 | 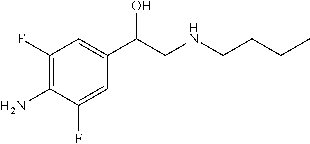 [SMILES: CCCCNCC(O)c1cc(F)c(N)c(F)c1] | 1-(4-amino-3,5- difluorophenyl)-2- (butylamino)-1-ethanol | +++ | +++ |
| 38 | 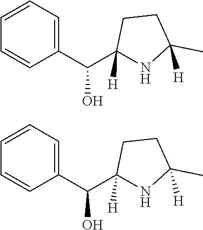 | Racemic mixture of (R)- [(2S,5S)-5-methyl-2- pyrrolidinyl]phenylmeth- anol and (S)-[(2R,5R)- 5-methyl-2- pyrrolidinyl]phenylmeth- anol | + | − |
| 39 | 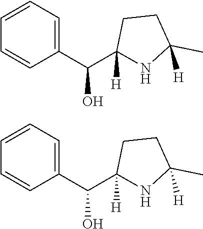 | Racemic mixture of (R)- [(2R,5R)-5-methyl-2- pyrrolidinyl]phenylmeth- anol and (S)-[(2S,5S)- 5-methyl-2- pyrrolidinyl]phenylmeth- anol | ++ | − |
| 40 | 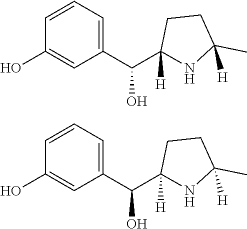 | Racemic mixture of m- {(R)-[(2S,5S)-5- methyl-2- pyrrolidinyl]hydroxymeth- yl}phenol and m-{(S)- [(2R,5R)-5-methyl-2- pyrrolidinyl]hydroxymeth- yl}phenol | ++ | − |
| 41 | 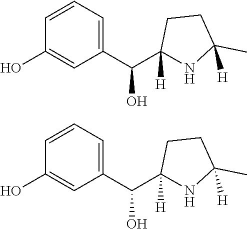 | Racemic mixture of m- {(R)-[(2R,5R)-5- methyl-2- pyrrolidinyl]hydroxymeth- yl}phenol and m-{(S)- [(2S,5S)-5-methyl-2- pyrrolidinyl]hydroxymeth- yl}phenol | +++ | − |
| 42 | 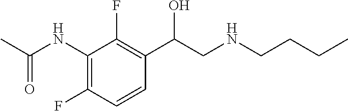 [SMILES: CCCCNCC(O)c1ccc(F)c(NC(C)=O)c1F] | N-{3-[2-(butylamino)- 1-hydroxyethyl]-2,6- difluorophenyl}acetamide | + | − |
| 43 | 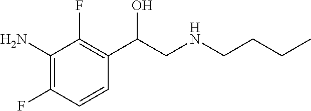 [SMILES: CCCCNCC(O)c1ccc(F)c(N)c1F] | 1-(3-amino-2,4- difluorophenyl)-2- (butylamino)-1-ethanol | +++ | − |
| 44 | 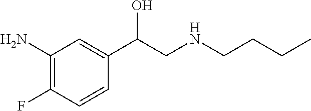 [SMILES: CCCCNCC(O)c1ccc(F)c(N)c1] | 1-(3-amino-4- fluorophenyl)-2- (butylamino)-1-ethanol | ++ | − |
| 45 | 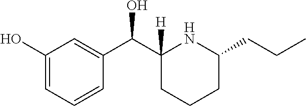 [SMILES: CCC[C@@H]1CCC[C@H]([C@H](O)c2cccc(O)c2)N1] | (R)-[(2R,6R)-6-propyl- 2-piperidyl](m- hydroxyphenyl)methanol | +++ | − |
| 46 | 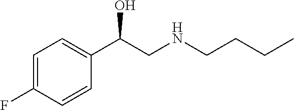 | (R)-2-(butylamino)-1- (p-fluorophenyl)-1- ethanol | ++ | − |
| 47 | 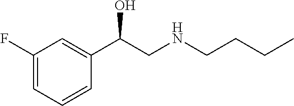 | (R)-2-(butylamino)-1- (m-fluorophenyl)-1- ethanol | ++ | − |
| 48 | 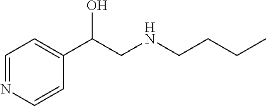 | 2-(butylamino)-1-(4- pyridyl)-1-ethanol | + | − |
| 49 | 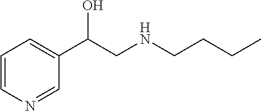 | 2-(butylamino)-1-(3- pyridyl)-1-ethanol | ++ | − |
| 50 | 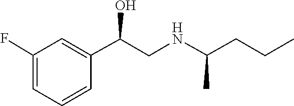 [SMILES: CCC[C@@H](C)NC[C@H](O)c1cccc(F)c1] | (R)-2-[(R)-1- methylbutylamino]-1- (m-fluorophenyl)-1- ethanol | +++ | − |
| 51 | 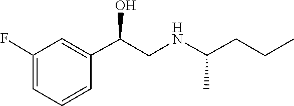 [SMILES: CCC[C@H](C)NC[C@H](O)c1cccc(F)c1] | (R)-2-[(S)-1- methylbutylamino]-1- (m-fluorophenyl)-1- ethanol | +++ | − |
| 52 | 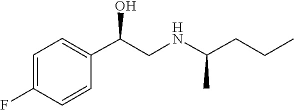 [SMILES: CCC[C@@H](C)NC[C@H](O)c1ccc(F)cc1] | (R)-2-[(R)-1- methylbutylamino]-1- (p-fluorophenyl)-1- ethanol | ++ | − |
| 53 | 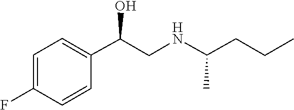 [SMILES: CCC[C@H](C)NC[C@H](O)c1ccc(F)cc1] | (R)-2-[(S)-1- methylbutylamino]-1- (p-fluorophenyl)-1- ethanol | ++ | − |
| 54 | 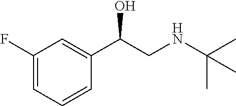 | (R)-2-(tert-butylamino)- 1-(m-fluorophenyl)-1- ethanol | +++ | − |
| 55 | 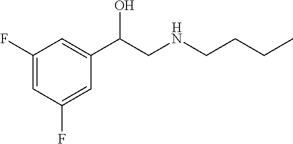 [SMILES: CCCCNCC(O)c1cc(F)cc(F)c1] | 2-(butylamino)-1-(3,5- difluorophenyl)-1- ethanol | ++ | − |
| 56 | 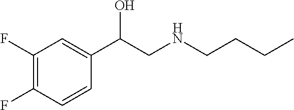 [SMILES: CCCCNCC(O)c1ccc(F)c(F)c1] | 2-(butylamino)-1-(3,4- difluorophenyl)-1- ethanol | ++ | − |
| 57 | 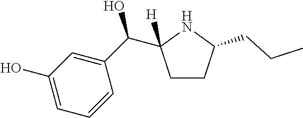 [SMILES: CCC[C@@H]1CC[C@H]([C@H](O)c2cccc(O)c2)N1] | m-{(R)-[(2R,5R)-5- propyl-2- pyrrolidinyl]hydroxymeth- yl}phenol | +++ | ++ |
| 58 | 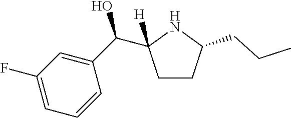 [SMILES: CCC[C@@H]1CC[C@H]([C@H](O)c2cccc(F)c2)N1] | (R)-[(2R,5R)-5-propyl- 2-pyrrolidinyl](m- fluorophenyl)methanol | +++ | + |
| 59 | 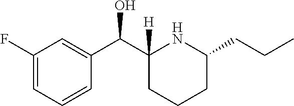 [SMILES: CCC[C@@H]1CCC[C@H]([C@H](O)c2cccc(F)c2)N1] | (R)-[(2R,6R)-6-propyl- 2-piperidyl](m- fluorophenyl)methanol | + | − |
| 60 | 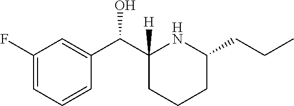 [SMILES: CCC[C@@H]1CCC[C@H]([C@@H](O)c2cccc(F)c2)N1] | (S)-[(2R,6R)-6-propyl- 2-piperidyl](m- fluorophenyl)methanol | + | − |
| 61 | 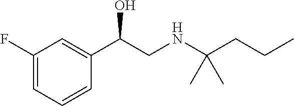 [SMILES: CCCC(C)(C)NC[C@H](O)c1cccc(F)c1] | (R)-2-(1,1- dimethylbutylamino)-1- (m-fluorophenyl)-1- ethanol | +++ | − |
| 62 | 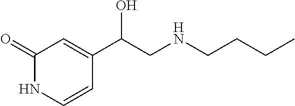 | 4-[2-(butylamino)-1- hydroyxethyl]-2(1H)- pyridinone | + | − |
| 63 | 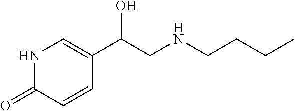 | 5-[2-(butylamino)-1- hydroxyethyl]-2(1H)- pyridinone | + | − |
| 64 | 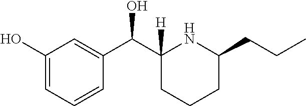 [SMILES: CCC[C@H]1CCC[C@H]([C@H](O)c2cccc(O)c2)N1] | (R)-[(2R,6S)-6-propyl- 2-piperidyl](m- hydroxyphenyl)methanol | +++ | − |
| 65 | 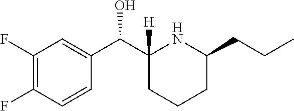 [SMILES: CCC[C@H]1CCC[C@H]([C@@H](O)c2ccc(F)c(F)c2)N1] | (S)-[(2R,6S)-6-propyl- 2-piperidyl](3,4- difluorophenyl)methanol | + | − |
| 66 | 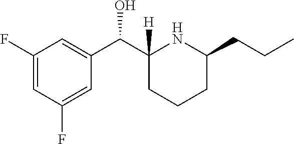 [SMILES: CCC[C@H]1CCC[C@H]([C@@H](O)c2cc(F)cc(F)c2)N1] | (S)-[(2R,6S)-6-propyl- 2-piperidyl](3,5- difluorophenyl)methanol | + | − |
| 67 | 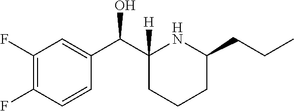 [SMILES: CCC[C@H]1CCC[C@H]([C@H](O)c2ccc(F)c(F)c2)N1] | (R)-[(2R,6S)-6-propyl- 2-piperidyl](3,4- difluorophenyl)methanol | + | − |
| 68 | 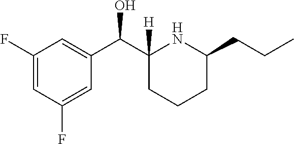 [SMILES: CCC[C@H]1CCC[C@H]([C@H](O)c2cc(F)cc(F)c2)N1] | (R)-[(2R,6S)-6-propyl- 2-piperidyl](3,5- difluorophenyl)methanol | ++ | − |
| 69 | 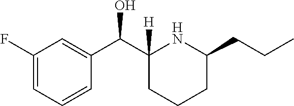 [SMILES: CCC[C@H]1CCC[C@H]([C@H](O)c2cccc(F)c2)N1] | (R)-[(2R,6S)-6-propyl- 2-piperidyl](m- fluorophenyl)methanol | ++ | − |
| 70 | 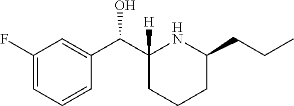 [SMILES: CCC[C@H]1CCC[C@H]([C@@H](O)c2cccc(F)c2)N1] | (S)-[(2R,6S)-6-propyl- 2-piperidyl](m- fluorophenyl)methanol | ++ | − |
| 71 | 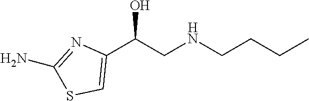 | (S)-1-(2-amino-1,3- thiazol-4-yl)-2- (butylamino)-1-ethanol | + | − |
| 72 | 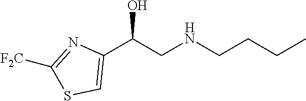 | (S)-2-(butylamino)-1- [2-(trifluoromethyl)- 1,3-thiazol-4-yl]-1- ethanol | + | − |
| 73 | 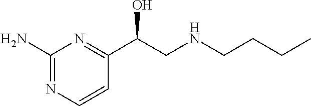 | (S)-1-(2-amino-4- pyrimidinyl)-2- (butylamino)-1-ethanol | + | − |
| 74 | 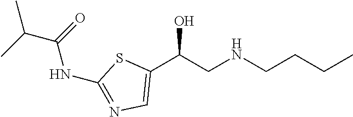 [SMILES: CCCCNC[C@H](O)c1cnc(NC(=O)C(C)C)s1] | N-{5-[(2S)-2- (butylamino)-1- hydroxyethyl]-1,3- thiazol-2-yl}2- methylpropionamide | + | − |
| 75 | 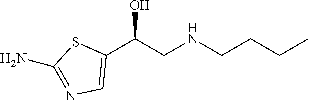 | (S)-1-(2-amino-1,3- thiazol-5-yl)-2- (butylamino)-1-ethanol | + | − |
| 76 | 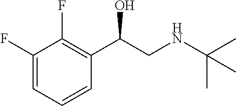 [SMILES: CC(C)(C)NC[C@H](O)c1cccc(F)c1F] | (R)-2-(tert-butylamino)- 1-(2,3-difluorophenyl)- 1-ethanol | +++ | + |
| 77 | 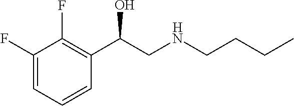 [SMILES: CCCCNC[C@H](O)c1cccc(F)c1F] | (R)-2-(butylamino)-1- (2,3-difluorophenyl)-1- ethanol | +++ | − |
| 78 | 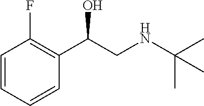 | (R)-2-(tert-butylamino)- 1-(o-fluorophenyl)-1- ethanol | +++ | + |
| 79 | 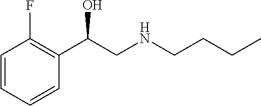 | (R)-2-(butylamino)-1- (o-fluorophenyl)-1- ethanol | +++ | − |
| 80 | 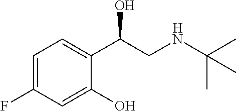 [SMILES: CC(C)(C)NC[C@H](O)c1ccc(F)cc1O] | (R)-2-(tert-butylamino)- 1-(4-fluoro-2- hydroxyphenyl)-1- ethanol | ++ | − |
| 81 | 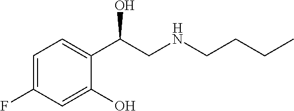 [SMILES: CCCCNC[C@H](O)c1ccc(F)cc1O] | (R)-2-(butylamino)-1- (4-fluoro-2- hydroxyphenyl)-1- ethanol | ++ | − |
| 82 | 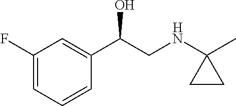 | (R)-1-(m-fluorophenyl)- 2-(1- methylcyclopropylamino)- 1-ethanol | +++ | − |
| 83 | 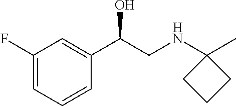 [SMILES: CC1(NC[C@H](O)c2cccc(F)c2)CCC1] | (R)-1-(m-fluorophenyl)- 2-(1- methylcyclobutylamino)- 1-ethanol | +++ | + |
| 84 | 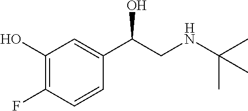 [SMILES: CC(C)(C)NC[C@H](O)c1ccc(F)c(O)c1] | (R)-2-(tert-butylamino)- 1-(4-fluoro-3- hydroxyphenyl)-1- ethanol | +++ | ++ |
| 85 | 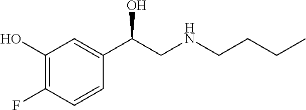 [SMILES: CCCCNC[C@H](O)c1ccc(F)c(O)c1] | (R)-2-(butylamino)-1- (4-fluoro-3- hydroxyphenyl)-1- ethanol | +++ | − |
| 86 | 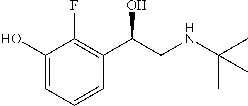 [SMILES: CC(C)(C)NC[C@H](O)c1cccc(O)c1F] | (R)-2-(tert-butylamino)- 1-(2-fluoro-3- hydroxyphenyl)-1- ethanol | ++ | ++ |
| 87 | 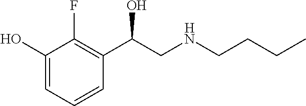 [SMILES: CCCCNC[C@H](O)c1cccc(O)c1F] | (R)-2-(butylamino)-1- (2-fluoro-3- hydroxyphenyl)-1- ethanol | +++ | + |
| 88 | 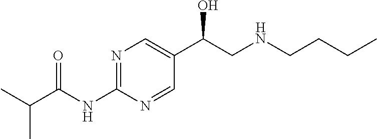 [SMILES: CCCCNC[C@H](O)c1cnc(NC(=O)C(C)C)nc1] | N-{5-[(R)-2- (butylamino)-1- hydroxyethyl]-2- pyrimidinyl}2- methylpropionamide | ++ | − |
| 89 | 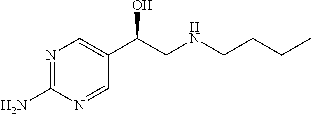 | (R)-1-(2-amino-5- pyrimidinyl)-2- (butylamino)-1-ethanol | ++ | − |
| 90 | 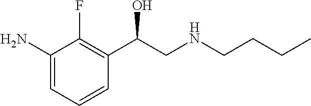 [SMILES: CCCCNC[C@H](O)c1cccc(N)c1F] | (R)-1-(3-amino-2- fluorophenyl)-2- (butylamino)-1-ethanol | ++ | − |
| 91 | 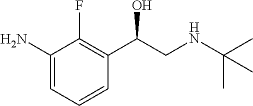 [SMILES: CC(C)(C)NC[C@H](O)c1cccc(N)c1F] | (R)-1-(3-amino-2- fluorophenyl)-2-(tert- butylamino)-1-ethanol | +++ | + |
| 92 | 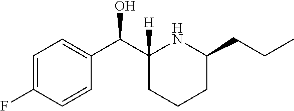 [SMILES: CCC[C@H]1CCC[C@H]([C@H](O)c2ccc(F)cc2)N1] | (R)-[(2R,6S)-6-propyl- 2-piperidyl](p- fluorophenyl)methanol | + | − |
| 93 | 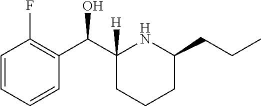 [SMILES: CCC[C@H]1CCC[C@H]([C@H](O)c2ccccc2F)N1] | (R)-[(2R,6S)-6-propyl- 2-piperidyl](o- fluorophenyl)methanol | + | − |
| 94 | 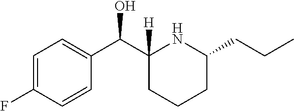 [SMILES: CCC[C@@H]1CCC[C@H]([C@H](O)c2ccc(F)cc2)N1] | (R)-[(2R,6R)-6-propyl- 2-piperidyl](p- fluorophenyl)methanol | ++ | − |
| 95 | 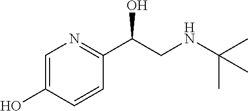 | 6-[(S)-2-(tert- butylamino)-1- hydroxyethyl]-3- pyridinol | ++ | − |
| 96 | 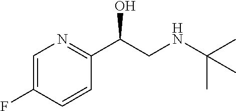 | (S)-2-(tert-butylamino)- 1-(5-fluoro-2-pyridyl)- 1-ethanol | ++ | − |
| 97 | 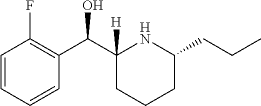 [SMILES: CCC[C@@H]1CCC[C@H]([C@H](O)c2ccccc2F)N1] | (R)-[(2R,6R)-6-propyl- 2-piperidyl](o- fluorophenyl)methanol | ++ | − |
| 98 | 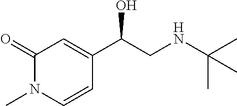 [SMILES: Cn1ccc([C@@H](O)CNC(C)(C)C)cc1=O] | 4-[(R)-2-(tert- butylamino)-1- hydroxyethyl]-1- methyl-2(1H)- pyridinone | +++ | − |
| 99 | 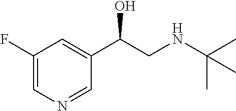 | (R)-2-(tert-butylamino)- 1-(5-fluoro-3-pyridyl)- 1-ethanol | +++ | + |
| 100 | 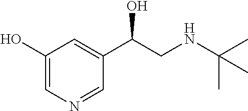 | 5-[(R)-2-(tert- butylamino)-1- hydroxyethyl]-3- pyridinol | +++ | − |
| 101 | 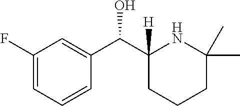 [SMILES: CC1(C)CCC[C@H]([C@@H](O)c2cccc(F)c2)N1] | (S)-[(R)-6,6-dimethyl- 2-piperidyl](m- fluorophenyl)methanol | + | − |
| 102 | 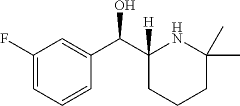 [SMILES: CC1(C)CCC[C@H]([C@H](O)c2cccc(F)c2)N1] | (R)-[(R)-6,6-dimethyl- 2-piperidyl](m- fluorophenyl)methanol | ++ | − |
| 103 | 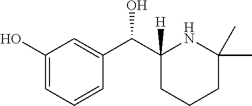 [SMILES: CC1(C)CCC[C@H]([C@@H](O)c2cccc(O)c2)N1] | (S)-[(R)-6,6-dimethyl- 2-piperidyl](m- hydroxyphenyl)methanol | + | − |
| 104 | 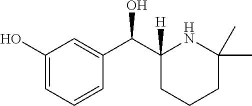 [SMILES: CC1(C)CCC[C@H]([C@H](O)c2cccc(O)c2)N1] | (R)-[(R)-6,6-dimethyl- 2-piperidyl](m- hydroxyphenyl)methanol | + | − |
| 105 | 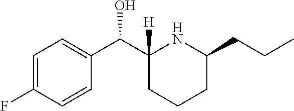 [SMILES: CCC[C@H]1CCC[C@H]([C@@H](O)c2ccc(F)cc2)N1] | (S)-[(2R,6S)-6-propyl- 2-piperidyl](p- fluorophenyl)methanol | + | − |
| 106 | 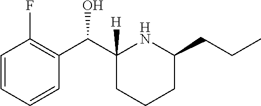 [SMILES: CCC[C@H]1CCC[C@H]([C@@H](O)c2ccccc2F)N1] | (S)-[(2R,6S)-6-propyl- 2-piperidyl](o- fluorophenyl)methanol | + | − |
| 107 | 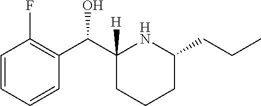 [SMILES: CCC[C@@H]1CCC[C@H]([C@@H](O)c2ccccc2F)N1] | (S)-[(2R,6R)-6-propyl- 2-piperidyl](o- fluorophenyl)methanol | + | − |
| 108 | 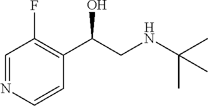 | (R)-2-(tert-butylamino)- 1-(3-fluoro-4-pyridyl)- 1-ethanol | + | − |
| 109 | 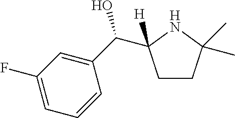 [SMILES: CC1(C)CC[C@H]([C@@H](O)c2cccc(F)c2)N1] | (S)-[(R)-5,5-dimethyl- 2-pyrrolidinyl](m- fluorophenyl)methanol | + | − |
| 110 | 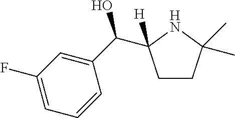 [SMILES: CC1(C)CC[C@H]([C@H](O)c2cccc(F)c2)N1] | (R)-[(R)-5,5-dimethyl- 2-pyrrolidinyl](m- fluorophenyl)methanol | +++ | − |
| 111 | 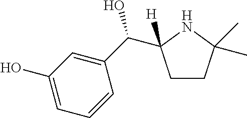 [SMILES: CC1(C)CC[C@H]([C@@H](O)c2cccc(O)c2)N1] | m-{(S)-[(R)-5,5- dimethyl-2- pyrrolidinyl]hydroxymeth- yl}phenol | ++ | − |
| 112 | 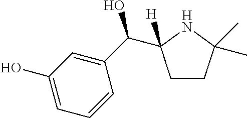 [SMILES: CC1(C)CC[C@H]([C@H](O)c2cccc(O)c2)N1] | m-{(R)-[(R)-5,5- dimethyl-2- pyrrolidinyl]hydroxymeth- yl}phenol | +++ | − |
| 113 | 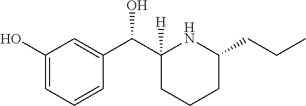 [SMILES: CCC[C@@H]1CCC[C@@H]([C@@H](O)c2cccc(O)c2)N1] | (S)-[(2S,6R)-6-propyl- 2-piperidyl](m- hydroxyphenyl)methanol | ++ | − |
| 114 | 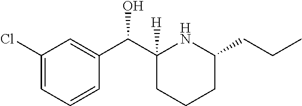 [SMILES: CCC[C@@H]1CCC[C@@H]([C@@H](O)c2cccc(Cl)c2)N1] | (S)-[(2S,6R)-6-propyl- 2-piperidyl](m- chlorophenyl)methanol | + | − |
| 115 | 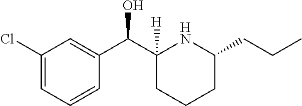 [SMILES: CCC[C@@H]1CCC[C@@H]([C@H](O)c2cccc(Cl)c2)N1] | (R)-[(2S,6R)-6-propyl- 2-piperidyl](m- chlorophenyl)methanol | +++ | − |
| 116 | 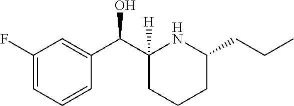 [SMILES: CCC[C@@H]1CCC[C@@H]([C@H](O)c2cccc(F)c2)N1] | (R)-[(2S,6R)-6-propyl- 2-piperidyl](m- fluorophenyl)methanol | + | − |
| 117 | 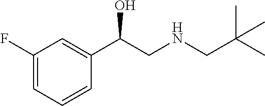 [SMILES: CC(C)(C)CNC[C@H](O)c1cccc(F)c1] | (R)-1-(m-flurophenyl)- 2-(neopentylamino)-1- ethanol | + | − |
| 118 | 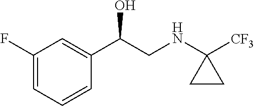 [SMILES: O[C@@H](CNC1(C(F)(F)F)CC1)c1cccc(F)c1] | (R)-1-(m-fluorophenyl)- 2-[1- (trifluoromethyl)cyclopro- pylamino]-1-ethanol | ++ | − |
| 119 | 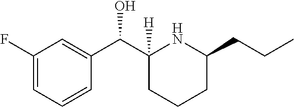 [SMILES: CCC[C@H]1CCC[C@@H]([C@@H](O)c2cccc(F)c2)N1] | (S)-[(2S,6S)-6-propyl- 2-piperidyl](m- fluorophenyl)methanol | + | − |
| 120 | 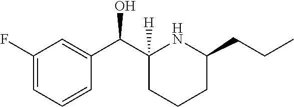 [SMILES: CCC[C@H]1CCC[C@@H]([C@H](O)c2cccc(F)c2)N1] | (R)-[(2S,6S)-6-propyl- 2-piperidyl](m- fluorophenyl)methanol | ++ | − |
| 121 | 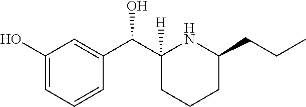 [SMILES: CCC[C@H]1CCC[C@@H]([C@@H](O)c2cccc(O)c2)N1] | (S)-[(2S,6S)-6-propyl- 2-piperidyl](m- hydroxyphenyl)methanol | +++ | − |
| 122 | 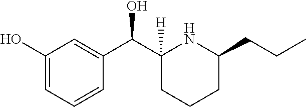 [SMILES: CCC[C@H]1CCC[C@@H]([C@H](O)c2cccc(O)c2)N1] | (R)-[(2S,6S)-6-propyl- 2-piperidyl](m- hydroxyphenyl)methanol | ++ | − |
| 123 | 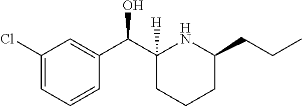 [SMILES: CCC[C@H]1CCC[C@@H]([C@H](O)c2cccc(Cl)c2)N1] | (R)-[(2S,6S)-6-propyl- 2-piperidyl](m- chlorophenyl)methanol | + | − |
| 124 | 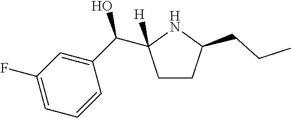 [SMILES: CCC[C@H]1CC[C@H]([C@H](O)c2cccc(F)c2)N1] | (R)-[(2R,5S)-5-propyl- 2-pyrrolidinyl](m- fluorophenyl)methanol | ++ | − |
| 125 | 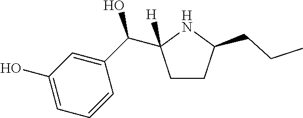 [SMILES: CCC[C@H]1CC[C@H]([C@H](O)c2cccc(O)c2)N1] | m-{(R)-[(2R,5S)-5- propyl-2- pyrrolidinyl]hydroxymeth- yl}phenol | ++ | − |
| 126 | 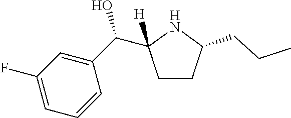 [SMILES: CCC[C@@H]1CC[C@H]([C@@H](O)c2cccc(F)c2)N1] | (S)-[(2R,5R)-5-propyl- 2-pyrrolidinyl](m- fluorophenyl)methanol | + | − |
| 127 | 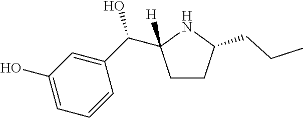 [SMILES: CCC[C@@H]1CC[C@H]([C@@H](O)c2cccc(O)c2)N1] | m-{(S)-[(2R,5R)-5- propyl-2- pyrrolidinyl]hydroxymeth- yl}phenol | + | − |
| 128 | 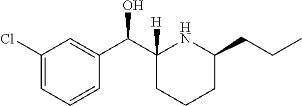 [SMILES: CCC[C@H]1CCC[C@H]([C@H](O)c2cccc(Cl)c2)N1] | (R)-[(2R,6S)-6-propyl- 2-piperidyl](m- chlorophenyl)methanol | + | − |
| 129 | 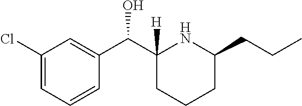 [SMILES: CCC[C@H]1CCC[C@H]([C@@H](O)c2cccc(Cl)c2)N1] | (S)-[(2R,6S)-6-propyl- 2-piperidyl](m- chlorophenyl)methanol | + | − |
| 130 | 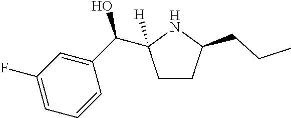 [SMILES: CCC[C@H]1CC[C@@H]([C@H](O)c2cccc(F)c2)N1] | (R)-[(2S,5R)-5-propyl- 2-pyrrolidinyl](m- fluorophenyl)methanol | + | − |
| 131 | 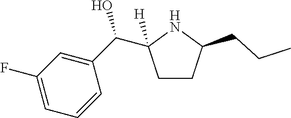 [SMILES: CCC[C@H]1CC[C@@H]([C@@H](O)c2cccc(F)c2)N1] | (S)-[(2S,5S)-5-propyl- 2-pyrrolidinyl](m- fluorophenyl)methanol | ++ | − |
| 132 | 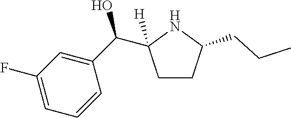 [SMILES: CCC[C@@H]1CC[C@@H]([C@H](O)c2cccc(F)c2)N1] | (R)-[(2S,5R)-5-propyl- 2-pyrrolidinyl](m- fluorophenyl)methanol | + | − |
| 133 | 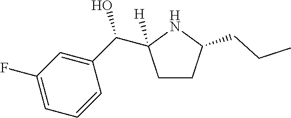 [SMILES: CCC[C@@H]1CC[C@@H]([C@@H](O)c2cccc(F)c2)N1] | (S)-[(2S,5R)-5-propyl- 2-pyrrolidinyl](m- fluorophenyl)methanol | ++ | − |
| 134 | 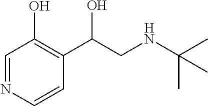 | 4-[2-(tert-butylamino)- 1-hydroxyethyl]-3- pyridinol | +++ | − |
| 135 | 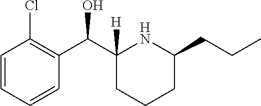 [SMILES: CCC[C@H]1CCC[C@H]([C@H](O)c2ccccc2Cl)N1] | (R)-[(2R,6S)-6-propyl- 2-piperidyl](o- chlorophenyl)methanol | + | − |
| 136 | 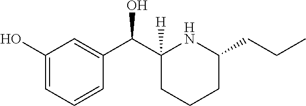 [SMILES: CCC[C@@H]1CCC[C@@H]([C@H](O)c2cccc(O)c2)N1] | (R)-[(2S,6R)-6-propyl- 2-piperidyl](m- hydroxyphenyl)methanol | +++ | − |
| 137 | 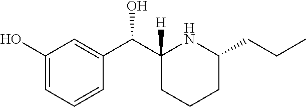 [SMILES: CCC[C@@H]1CCC[C@H]([C@@H](O)c2cccc(O)c2)N1] | (S)-[(2R,6R)-6-propyl- 2-piperidyl](m- hydroxyphenyl)methanol | + | − |
| 138 | 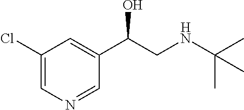 | (R)-2-(tert-butylamino)- 1-(5-chloro-3-pyridyl)- 1-ethanol | +++ | +++ |
| 139 | 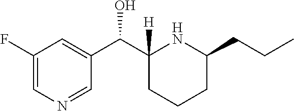 [SMILES: CCC[C@H]1CCC[C@H]([C@@H](O)c2cncc(F)c2)N1] | (S)-[(2R,6S)-6-propyl- 2-piperidyl](5-fluoro-3- pyridyl)methanol | − | − |
| 140 | 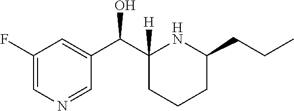 [SMILES: CCC[C@H]1CCC[C@H]([C@H](O)c2cncc(F)c2)N1] | (R)-[(2R,6S)-6-propyl- 2-piperidyl](5-fluoro-3- pyridyl)methanol | − | − |
| 141 | 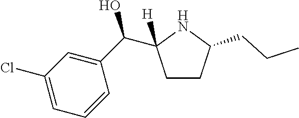 [SMILES: CCC[C@@H]1CC[C@H]([C@H](O)c2cccc(Cl)c2)N1] | (R)-[(2R,5R)-5-propyl- 2-pyrrolidinyl](m- chlorophenyl)methanol | ++ | − |
| 142 | 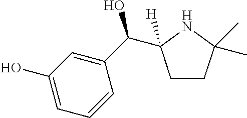 [SMILES: CC1(C)CC[C@@H]([C@H](O)c2cccc(O)c2)N1] | m-{(R)-[(S)-5,5- dimethyl-2- pyrrolidinyl]hydroxymeth- yl}phenol | + | − |
| 143 | 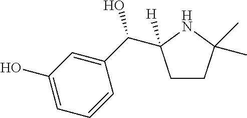 [SMILES: CC1(C)CC[C@@H]([C@@H](O)c2cccc(O)c2)N1] | m-{(S)-[(S)-5,5- dimethyl-2- pyrrolidinyl]hydroxymeth- yl}phenol | − | − |
| 144 | 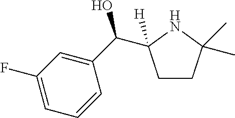 [SMILES: CC1(C)CC[C@@H]([C@H](O)c2cccc(F)c2)N1] | (R)-[(S)-5,5-dimethyl- 2-pyrrolidinyl](m- fluorophenyl)methanol | − | − |
| 145 | 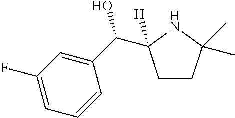 [SMILES: CC1(C)CC[C@@H]([C@@H](O)c2cccc(F)c2)N1] | (S)-[(S)-5,5-dimethyl- 2-pyrrolidinyl](m- fluorophenyl)methanol | + | − |
| 146 | 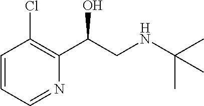 | (S)-2-(tert-butylamino)- 1-(3-chloro-2-pyridyl)- 1-ethanol | + | − |
| 147 | 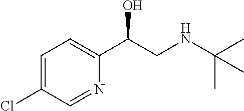 | (S)-2-(tert-butylamino)- 1-(5-chloro-2-pyridyl)- 1-ethanol | + | − |
| 148 | 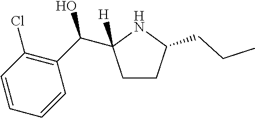 [SMILES: CCC[C@@H]1CC[C@H]([C@H](O)c2ccccc2Cl)N1] | (R)-[(2R,5R)-5-propyl- 2-pyrrolidinyl](o- chlorophenyl)methanol | +++ | − |
| 149 | 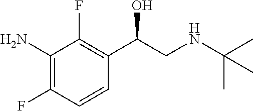 [SMILES: CC(C)(C)NC[C@H](O)c1ccc(F)c(N)c1F] | (R)-1-(3-amino-2,4- difluorophenyl)-2-(tert- butylamino)-1-ethanol | +++ | + |
| 150 | 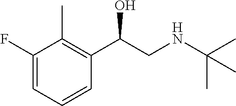 [SMILES: Cc1c(F)cccc1[C@@H](O)CNC(C)(C)C] | (R)-2-(tert-butylamino)- 1-(3-fluoro-2-tolyl)-1- ethanol | +++ | + |
| 151 | 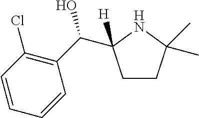 [SMILES: CC1(C)CC[C@H]([C@@H](O)c2ccccc2Cl)N1] | (S)-[(R)-5,5-dimethyl- 2-pyrrolidinyl](o- chlorophenyl)methanol | − | − |
| 152 | 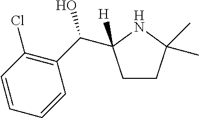 [SMILES: CC1(C)CC[C@H]([C@@H](O)c2ccccc2Cl)N1] | (R)-[(R)-5,5-dimethyl- 2-pyrrolidinyl](o- chlorophenyl)methanol | +++ | − |
| 153 | 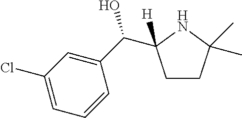 [SMILES: CC1(C)CC[C@H]([C@@H](O)c2cccc(Cl)c2)N1] | (S)-[(R)-5,5-dimethyl- 2-pyrrolidinyl](m- chlorophenyl)methanol | + | − |
| 154 | 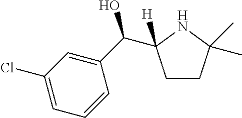 [SMILES: CC1(C)CC[C@H]([C@H](O)c2cccc(Cl)c2)N1] | (R)-[(R)-5,5-dimethyl- 2-pyrrolidinyl](m- chlorophenyl)methanol | +++ | − |
| 155 | 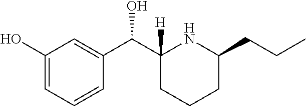 [SMILES: CCC[C@H]1CCC[C@H]([C@@H](O)c2cccc(O)c2)N1] | (S)-[(2R,6S)-6-propyl- 2-piperidyl](m- hydroxyphenyl)methanol | − | − |
| 156 | 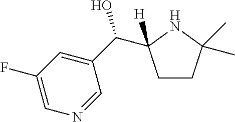 [SMILES: CC1(C)CC[C@H]([C@@H](O)c2cncc(F)c2)N1] | (S)-[(R)-5,5-dimethyl- 2-pyrrolidinyl](5-fluoro- 3-pyridyl)methanol | − | − |
| 157 | 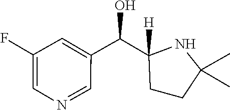 [SMILES: CC1(C)CC[C@H]([C@H](O)c2cncc(F)c2)N1] | (R)-[(R)-5,5-dimethyl- 2-pyrrolidinyl](5-fluoro- 3-pyridyl)methanol | +++ | − |
| 158 | 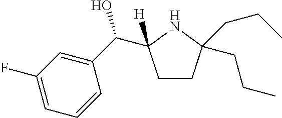 [SMILES: CCCC1(CCC)CC[C@H]([C@@H](O)c2cccc(F)c2)N1] | (S)-[(R)-5,5-dipropyl-2- pyrrolidinyl](m- fluorophenyl)methanol | − | − |
| 159 | 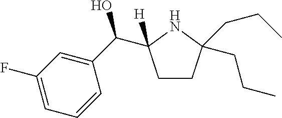 [SMILES: CCCC1(CCC)CC[C@H]([C@H](O)c2cccc(F)c2)N1] | (R)-[(R)-5,5-dipropyl-2- pyrrolidinyl](m- fluorophenyl)methanol | ++ | − |
| 160 | 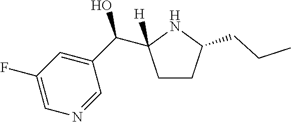 [SMILES: CCC[C@@H]1CC[C@H]([C@H](O)c2cncc(F)c2)N1] | (R)-[(2R,5R)-5-propyl- 2-pyrrolidinyl](5-fluoro- 3-pyridyl)methanol | +++ | − |
| 161 | 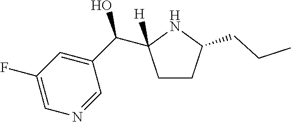 [SMILES: CCC[C@@H]1CC[C@H]([C@H](O)c2cncc(F)c2)N1] | (S)-[(2R,5R)-5-propyl- 2-pyrrolidinyl](5-fluoro- 3-pyridyl)methanol | + | − |
| 162 | 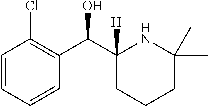 [SMILES: CC1(C)CCC[C@H]([C@H](O)c2ccccc2Cl)N1] | (R)-[(R)-6,6-dimethyl- 2-piperidyl](o- chlorophenyl)methanol | + | − |
| 163 | 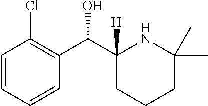 [SMILES: CC1(C)CCC[C@H]([C@@H](O)c2ccccc2Cl)N1] | (S)-[(R)-6,6-dimethyl- 2-piperidyl](o- chlorophenyl)methanol | +++ | − |
| 164 | 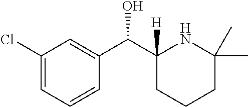 [SMILES: CC1(C)CCC[C@H]([C@@H](O)c2cccc(Cl)c2)N1] | (S)-[(R)-6,6-dimethyl- 2-piperidyl](m- chlorophenyl)methanol | ++ | − |
| 165 | 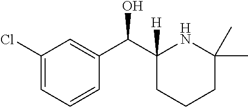 [SMILES: CC1(C)CCC[C@H]([C@H](O)c2cccc(Cl)c2)N1] | (R)-[(R)-6,6-dimethyl- 2-piperidyl](m- chlorophenyl)methanol | + | − |
| 166 | 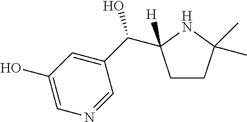 [SMILES: CC1(C)CC[C@H]([C@@H](O)c2cncc(O)c2)N1] | 5-{(S)-[(R)-5,5- dimethyl-2- pyrrolidinyl]hydroxymeth- yl}-3-pyridinol | + | − |
| 167 | 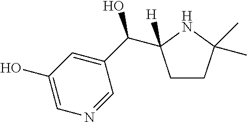 [SMILES: CC1(C)CC[C@H]([C@H](O)c2cncc(O)c2)N1] | 5-{(R)-[(R)-5,5- dimethyl-2- pyrrolidinyl]hydroxymeth- yl}-3-pyridinol | ++ | − |
| 168 | 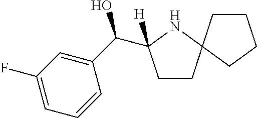 [SMILES: O[C@H](c1cccc(F)c1)[C@H]1CCC2(CCCC2)N1] | (R)-{(R)-1-aza-2- spiro[5.5]non}(m- fluorophenyl)methanol | ++ | + |
| 169 | 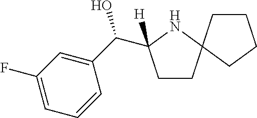 [SMILES: O[C@@H](c1cccc(F)c1)[C@H]1CCC2(CCCC2)N1] | (S)-{(R)-1-aza-2- spiro[4.4]nonyl}(m- fluorophenyl)methanol | − | − |
| 170 | 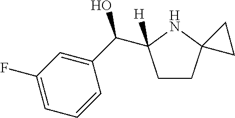 [SMILES: O[C@H](c1cccc(F)c1)[C@H]1CCC2(CC2)N1] | (R)-{(R)-4-aza-5- spiro[2.4]heptyl}(m- fluorophenyl)methanol | ++ | − |
| 171 | 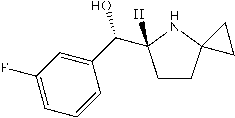 [SMILES: O[C@@H](c1cccc(F)c1)[C@H]1CCC2(CC2)N1] | (S)-{(R)-4-aza-5- spiro[2.4]heptyl}(m- fluorophenyl)methanol | + | − |
| 172 | 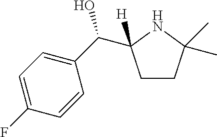 [SMILES: CC1(C)CC[C@H]([C@@H](O)c2ccc(F)cc2)N1] | (S)-[(R)-5,5-dimethyl- 2-pyrrolidinyl](p- fluorophenyl)methanol | − | − |
| 173 | 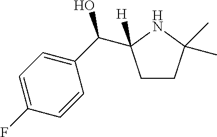 [SMILES: CC1(C)CC[C@H]([C@H](O)c2ccc(F)cc2)N1] | (R)-[(R)-5,5-dimethyl- 2-pyrrolidinyl](p- fluorophenyl)methanol | ++ | − |
| 174 | 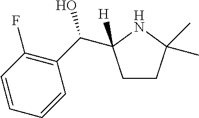 [SMILES: CC1(C)CC[C@H]([C@@H](O)c2ccccc2F)N1] | (S)-[(R)-5,5-dimethyl- 2-pyrrolidinyl](o- fluorophenyl)methanol | − | − |
| 175 | 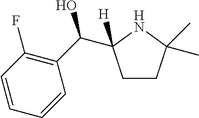 [SMILES: CC1(C)CC[C@H]([C@H](O)c2ccccc2F)N1] | (R)-[(R)-5,5-dimethyl- 2-pyrrolidinyl](o- fluorophenyl)methanol | +++ | + |
| 176 | 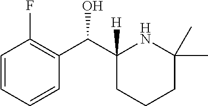 [SMILES: CC1(C)CCC[C@H]([C@@H](O)c2ccccc2F)N1] | (S)-[(R)-6,6-dimethyl- 2-piperidyl](o- fluorophenyl)methanol | +++ | + |
| 177 | 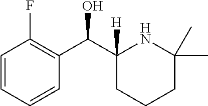 [SMILES: CC1(C)CCC[C@H]([C@H](O)c2ccccc2F)N1] | (R)-[(R)-6,6-dimethyl- 2-piperidyl](o- fluorophenyl)methanol | ++ | − |
| 178 | 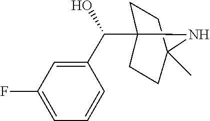 [SMILES: CC12CCC([C@@H](O)c3cccc(F)c3)(CC1)N2] | (S)-(m-fluorophenyl)(4- methyl-7- azabicyclo[2.2.1]hept- 1-yl)methanol | + | − |
| 179 | 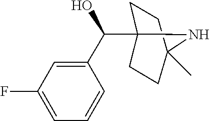 [SMILES: CC12CCC([C@H](O)c3cccc(F)c3)(CC1)N2] | (R)-(m-fluoropehnyl)(4- methyl-7- azabicyclo[2.2.1]hept- 1-yl)methanol | ++ | − |
| 180 | 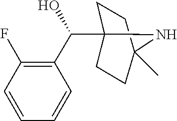 [SMILES: CC12CCC([C@@H](O)c3ccccc3F)(CC1)N2] | (S)-(o-fluorophenyl)(4- methyl-7- azabicyclo[2.2.1]hept- 1-yl)methanol | +++ | − |
| 181 | 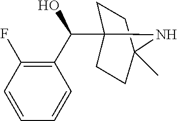 [SMILES: CC12CCC([C@H](O)c3ccccc3F)(CC1)N2] | (R)-(o-fluorophenyl)(4- methyl-7- azabicyclo[2.2.1]hept- 1-yl)methanol | +++ | − |
| 182 | 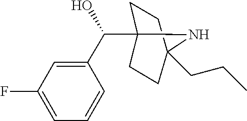 [SMILES: CCCC12CCC([C@@H](O)c3cccc(F)c3)(CC1)N2] | (S)-(m-fluorophenyl)(- propyl-7- azabicyclo[2.2.1]hept- 1-yl)methanol − | − | − |
| 183 | 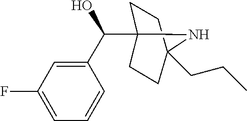 [SMILES: CCCC12CCC([C@H](O)c3cccc(F)c3)(CC1)N2] | (R)-(m-fluorophenyl)(4- propyl-7- azabicyclo[2.2.1]hept- 1-yl)methanol | ++ | + |
| 184 | 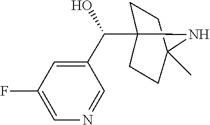 [SMILES: CC12CCC([C@@H](O)c3cncc(F)c3)(CC1)N2] | (S)-(5-fluoro-3- pyridyl)(4-methyl-7- azabicyclo[2.2.1]hept- 1-yl)methanol | ++ | − |
| 185 | 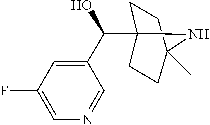 [SMILES: CC12CCC([C@H](O)c3cncc(F)c3)(CC1)N2] | (R)-(5-fluoro-3- pyridyl)(4-methyl-7- azabicyclo[2.2.1]hept- 1-yl)methanol | +++ | − |
| 186 | 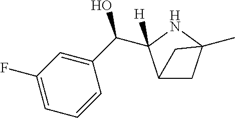 [SMILES: CC12CC(C1)[C@H]([C@H](O)c1cccc(F)c1)N2] | (R)-{(R)-1-methyl-2- azabicyclo[2.1.1]hex-3- yl}(m- fluorophenyl)methanol | +++ | ++ |
| 187 | 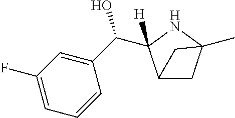 [SMILES: CC12CC(C1)[C@H]([C@@H](O)c1cccc(F)c1)N2] | (S)-{(R)-1-methyl-2- azabicyclo[2.1.1]hex-3- yl}(m- fluorophenyl)methanol | + | − |
| 188 | 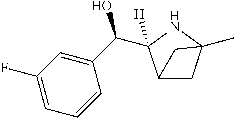 [SMILES: CC12CC(C1)[C@@H]([C@H](O)c1cccc(F)c1)N2] | (R)-{(S)-1-methyl-2- azabicyclo[2.1.1]hex-3- yl}(m- fluorophenyl)methanol | − | − |
| 189 | 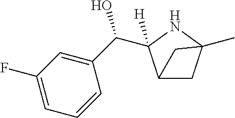 [SMILES: CC12CC(C1)[C@@H]([C@@H](O)c1cccc(F)c1)N2] | (S)-{(S)-1-methyl-2- azabicyclo[2.1.1]hex-3- yl}(m- fluorophenyl)methanol | ++ | − |
| 190 | 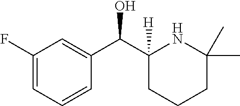 [SMILES: CC1(C)CCC[C@@H]([C@H](O)c2cccc(F)c2)N1] | (R)-[(S)-6,6-dimethyl- 2-piperidyl](m- fluorophenyl)methanol | +++ | − |
| 191 | 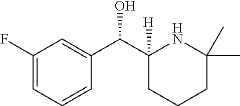 [SMILES: CC1(C)CCC[C@@H]([C@@H](O)c2cccc(F)c2)N1] | (S)-[(S)-6,6-dimethyl- 2-piperidyl](m- fluorophenyl)methanol | + | − |
| 192 | 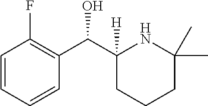 [SMILES: CC1(C)CCC[C@@H]([C@@H](O)c2ccccc2F)N1] | (S)-[(S)-6,6-dimethyl- 2-piperidyl](o- fluorophenyl)methanol | + | − |
| 193 | 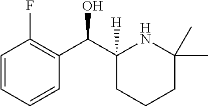 [SMILES: CC1(C)CCC[C@@H]([C@H](O)c2ccccc2F)N1] | (R)-[(S)-6,6-dimethyl- 2-piperidyl](o- fluorophenyl)methanol | +++ | + |
| 194 | 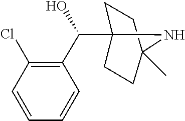 [SMILES: CC12CCC([C@@H](O)c3ccccc3Cl)(CC1)N2] | (S)-(o-chlorophenyl)(4- methyl-7- azabicyclo[2.2.1]hept- 1-yl)methanol | +++ | − |
| 195 | 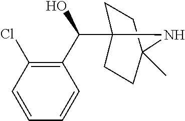 [SMILES: CC12CCC([C@H](O)c3ccccc3Cl)(CC1)N2] | (R)-(o-chlorophenyl)(4- methyl-7- azabicyclo[2.2.1]hept- 1-yl)methanol | +++ | − |
| 196 | 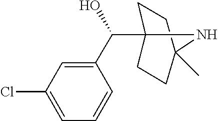 [SMILES: CC12CCC([C@@H](O)c3cccc(Cl)c3)(CC1)N2] | (S)-(m-chlorophenyl)(4- methyl-7- azabicyclo[2.2.1]hept- 1-yl)methanol | + | − |
| 197 | 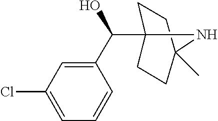 [SMILES: CC12CCC([C@H](O)c3cccc(Cl)c3)(CC1)N2] | (R)-(m-chlorophenyl)(4- methyl-7- azabicyclo[2.2.1]hept- 1-yl)methanol | +++ | − |
| 198 | 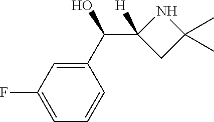 | (R)-[(R)-4,4-dimethyl- 2-azetidinyl](m- fluorophenyl)methanol | +++ | + |
| 199 | 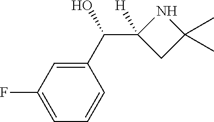 | (S)-[(S)-4,4-dimethyl- 2-azetidinyl](m- fluorophenyl)methanol | + | − |
| 200 | 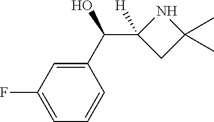 | (R)-[(S)-4,4-dimethyl- 2-azetidinyl](m- fluorophenyl)methanol | − | − |
| 201 | 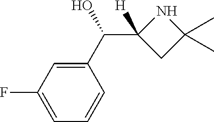 | (S)-[(R)-4,4-dimethyl- 2-azetidinyl](m- fluorophenyl)methanol | − | − |
| 202 | 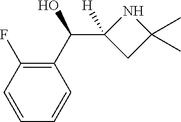 | (R)-[(S)-4,4-dimethyl- 2-azetidinyl](o- fluorophenyl)methanol | − | − |
| 203 | 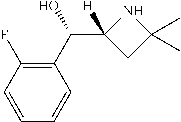 | (S)-[(R)-4,4-dimethyl- 2-azetidinyl](o- fluorophenyl)methanol | + | − |
| 204 | 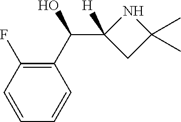 | (R)-[(R)-4,4-dimethyl- 2-azetidinyl](o- fluorophenyl)methanol | +++ | + |
| 205 | 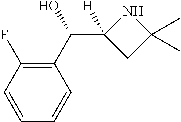 | (S)-[(S)-4,4-dimethyl- 2-azetidinyl](o- fluorophenyl)methanol | + | − |
| 206 | 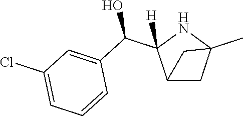 [SMILES: CC12CC(C1)[C@H]([C@H](O)c1cccc(Cl)c1)N2] | (R)-{(R)-1-methyl-2- azabicyclo[2.1.1]hex-3- yl}(m- chlorophenyl)methanol | +++ | − |
| 207 | 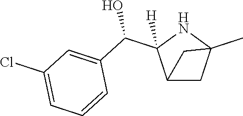 [SMILES: CC12CC(C1)[C@@H]([C@@H](O)c1cccc(Cl)c1)N2] | (S)-{(S)-1-methyl-2- azabicyclo[2.1.1]hex-3- yl}(m- chlorophenyl)methanol | + | − |
| 208 | 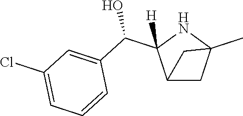 [SMILES: CC12CC(C1)[C@H]([C@@H](O)c1cccc(Cl)c1)N2] | (S)-{(R)-1-methyl-2- azabicyclo[2.1.1]hex-3- yl}(m- chlorophenyl)methanol | ++ | − |
| 209 | 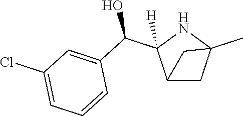 [SMILES: CC12CC(C1)[C@@H]([C@H](O)c1cccc(Cl)c1)N2] | (R)-{(S)-1-methyl-2- azabicyclo[2.1.1]hex-3- yl}(m- chlorophenyl)methanol | + | − |
| 210 | 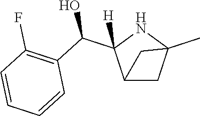 [SMILES: CC12CC(C1)[C@H]([C@H](O)c1ccccc1F)N2] | (R)-{(R)-1-methyl-2- azabicyclo[2.1.1]hex-3- yl}(o- fluorophenyl)methanol | +++ | +++ |
| 211 | 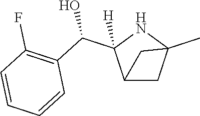 [SMILES: CC12CC(C1)[C@@H]([C@@H](O)c1ccccc1F)N2] | (S)-{(S)-1-methyl-2- azabicyclo[2.1.1]hex-3- yl}(o- fluorophenyl)methanol | +++ | − |
| 212 | 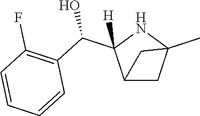 [SMILES: CC12CC(C1)[C@H]([C@@H](O)c1ccccc1F)N2] | (S)-{(R)-1-methyl-2- azabicyclo[2.1.1]hex-3- yl}(o- fluorophenyl)methanol | +++ | − |
| 213 | 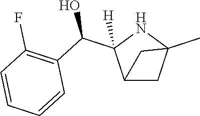 [SMILES: CC12CC(C1)[C@@H]([C@H](O)c1ccccc1F)N2] | (R)-{(S)-1-methyl-2- azabicyclo[2.1.1]hex-3- yl}(o- fluorophenyl)methanol | +++ | − |
| 214 | 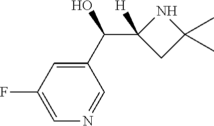 | (R)-[(R)-4,4-dimethyl- 2-azetidinyl](5-fluoro-3- pyridyl)methanol | +++ | − |
| 215 | 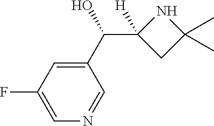 | (S)-[(S)-4,4-dimethyl- 2-azetidinyl](5-fluoro-3- pyridyl)methanol | + | − |
| 216 | 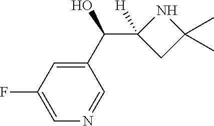 | (R)-[(S)-4,4-dimethyl- 2-azetidinyl](5-fluoro-3- pyridyl)methanol | + | − |
| 217 | 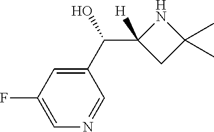 | (S)-[(R)-4,4-dimethyl- 2-azetidinyl](5-fluoro-3- pyridyl)methano | + | − |
| 218 | 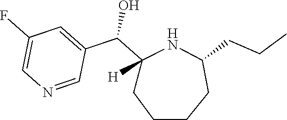 [SMILES: CCC[C@@H]1CCCC[C@H]([C@@H](O)c2cncc(F)c2)N1] | (S)-[(2R,7R)-7-propyl- 2-azepanyl](5-fluoro-3- pyridyl)methanol | − | − |
| 219 | 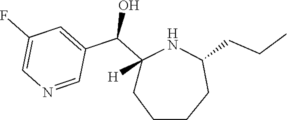 [SMILES: CCC[C@@H]1CCCC[C@H]([C@H](O)c2cncc(F)c2)N1] | (R)-[(2R,7R)-7-propyl- 2-azepanyl](5-fluoro-3- pyridyl)methanol | − | − |
| 220 | 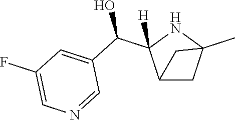 [SMILES: CC12CC(C1)[C@H]([C@H](O)c1cncc(F)c1)N2] | (R)-{(R)-1-methyl-2- azabicyclo[2.1.1]hex-3- yl}(5-fluoro-3- pyridyl)methanol | +++ | − |
| 221 | 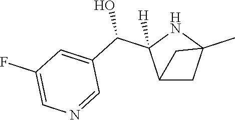 [SMILES: CC12CC(C1)[C@@H]([C@@H](O)c1cncc(F)c1)N2] | (S)-{(S)-1-methyl-2- azabicyclo[2.1.1]hex-3- yl}(5-fluoro-3- pyridyl)methanol | ++ | − |
| 222 | 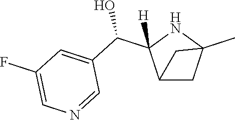 [SMILES: CC12CC(C1)[C@H]([C@@H](O)c1cncc(F)c1)N2] | (S)-{(R)-1-methyl-2- azabicyclo[2.1.1]hex-3- yl}(5-fluoro-3- pyridyl)methanol | − | − |
| 223 | 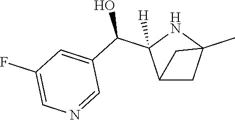 [SMILES: CC12CC(C1)[C@@H]([C@H](O)c1cncc(F)c1)N2] | (R)-{(S)-1-methyl-2- azabicyclo[2.1.1]hex-3- yl}(5-fluoro-3- pyridyl)methanol | + | − |
| 224 | 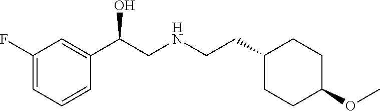 [SMILES: CO[C@H]1CC[C@H](CCNC[C@H](O)c2cccc(F)c2)CC1] | (R)-1-(m-fluorophenyl)- 2-{2-[(1r,4R)-4- methoxycyclohexyl]ethyl- amino}-1-ethanol | + | − |
| 225 | 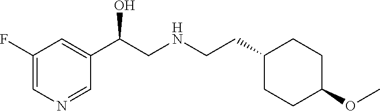 [SMILES: CO[C@H]1CC[C@H](CCNC[C@H](O)c2cncc(F)c2)CC1] | (R)-1-(5-fluoro-3- pyridyl)-2-{2-[(1r,4R)- 4- methoxycyclohexyl]ethyl- amino}-1-ethanol | ++ | − |
| 226 | 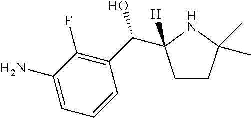 [SMILES: CC1(C)CC[C@H]([C@@H](O)c2cccc(N)c2F)N1] | (S)-[(R)-5,5-dimethyl- 2-pyrrolidinyl](3-amino- 2- fluorophenyl)methanol | + | − |
| 227 | 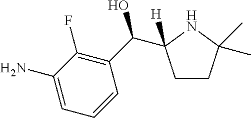 [SMILES: CC1(C)CC[C@H]([C@H](O)c2cccc(N)c2F)N1] | (R)-[(R)-5,5-dimethyl- 2-pyrrolidinyl](3-amino- 2- fluorophenyl)methanol | +++ | − |
| 228 | 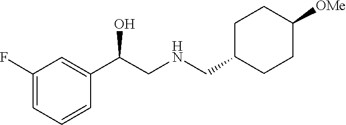 [SMILES: CO[C@H]1CC[C@H](CNC[C@H](O)c2cccc(F)c2)CC1] | (R)-1-(m-fluorophenyl)- 2-({[(1r,4R)-4- methoxycyclohexyl]meth- yl}amino)-1-ethanol | ++ | − |
| 229 | 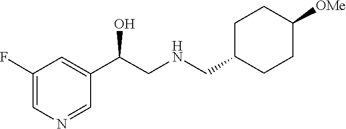 [SMILES: CO[C@H]1CC[C@H](CNC[C@H](O)c2cncc(F)c2)CC1] | (R)-1-(5-fluoro-3- pyridyl)-2-({[(1r,4R)-4- methoxycyclohexyl]meth- yl}amino)-1-ethanol | ++ | − |
| 230 | 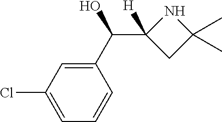 | (R)-[(R)-4,4-dimethyl- 2-azetidinyl](m- chlorophenyl)methanol | +++ | − |
| 231 | 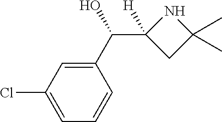 | (S)-[(S)-4,4-dimethyl- 2-azetidinyl](m- chlorophenyl)methanol | + | − |
| 232 | 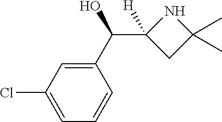 | (R)-[(S)-4,4-dimethyl- 2-azetidinyl](m- chlorophenyl)methanol | + | − |
| 233 | 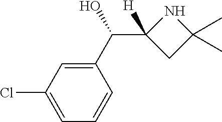 | (S)-[(R)-4,4-dimethyl- 2-azetidinyl](m- chlorophenyl)methanol | − | − |
| 234 | 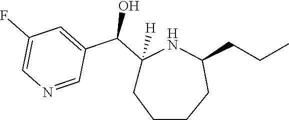 [SMILES: CCC[C@H]1CCCC[C@@H]([C@H](O)c2cncc(F)c2)N1] | (R)-[(2S,7S)-7-propyl- 2-azepanyl](5-fluoro-3- pyridyl)methanol | +++ | − |
| 235 | 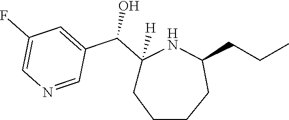 [SMILES: CCC[C@H]1CCCC[C@@H]([C@@H](O)c2cncc(F)c2)N1] | (S)-[(2S,7S)-7-propyl- 2-azepanyl](5-fluoro-3- pyridyl)methanol | − | − |
| 236 | 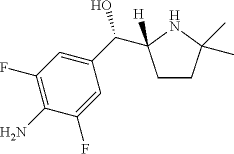 [SMILES: CC1(C)CC[C@H]([C@@H](O)c2cc(F)c(N)c(F)c2)N1] | (S)-[(R)-5,5-dimethyl- 2-pyrrolidinyl](4-amino- 3,5- difluorophenyl)methanol | +++ | + |
| 237 | 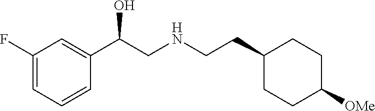 [SMILES: CO[C@H]1CC[C@@H](CCNC[C@H](O)c2cccc(F)c2)CC1] | (R)-1-(m-fluorophenyl)- 2-{2-[(1s,4S)-4- methoxycyclohexyl]ethyl- amino}-1-ethanol | ++ | − |
| 238 | 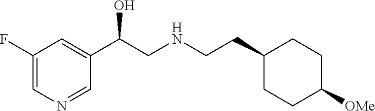 [SMILES: CO[C@H]1CC[C@@H](CCNC[C@H](O)c2cncc(F)c2)CC1] | (R)-1-(5-fluoro-3- pyridyl)-2-{2-[(1s,4S)- 4- methoxycyclohexyl]ethyl- amino}-1-ethanol | +++ | − |
| 239 | 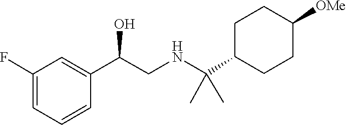 [SMILES: CO[C@H]1CC[C@H](C(C)(C)NC[C@H](O)c2cccc(F)c2)CC1] | (R)-1-(m-fluorophenyl)- 2-{1-methyl-1- [(1r,4R)-4- methoxycyclohexyl]ethyl- amino}-1-ethanol | +++ | − |
| 240 | 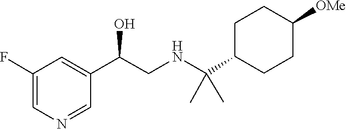 [SMILES: CO[C@H]1CC[C@H](C(C)(C)NC[C@H](O)c2cncc(F)c2)CC1] | (R)-1-(5-fluoro-3- pyridyl)-2-{1-methyl-1- [(1r,4R)-4- methoxycyclohexyl]ethyl- amino}-1-ethanol | +++ | − |
| 241 | 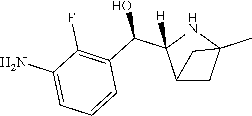 [SMILES: CC12CC(C1)[C@H]([C@H](O)c1cccc(N)c1F)N2] | (R)-{(R)-1-methyl-2- azabicyclo[2.1.1]hex-3- yl}(3-amino-2- fluorophenyl)methanol | +++ | − |
| 242 | 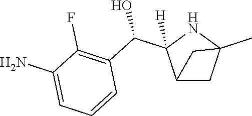 [SMILES: CC12CC(C1)[C@@H]([C@@H](O)c1cccc(N)c1F)N2] | (S)-{(S)-1-methyl-2- azabicyclo[2.1.1]hex-3- yl}(3-amino-2- fluorophenyl)methanol | ++ | − |
| 243 | 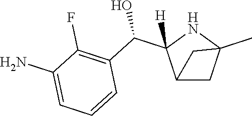 [SMILES: CC12CC(C1)[C@H]([C@@H](O)c1cccc(N)c1F)N2] | (S)-{(R)-1-methyl-2- azabicyclo[2.1.1]hex-3- yl}(3-amino-2- fluorophenyl)methanol | ++ | − |
| 244 | 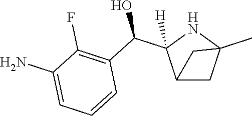 [SMILES: CC12CC(C1)[C@@H]([C@H](O)c1cccc(N)c1F)N2] | (R)-{(S)-1-methyl-2- azabicyclo[2.1.1]hex-3- yl}(3-amino-2- fluorophenyl)methanol | ++ | − |
| 245 | 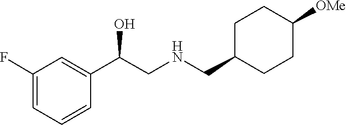 [SMILES: CO[C@H]1CC[C@@H](CNC[C@H](O)c2cccc(F)c2)CC1] | (R)-1-(m-fluorophenyl)- 2-({[(1s,4S)-4- methoxycyclohexyl]meth- yl}amino)-1-ethanol | ++ | − |
| 246 | 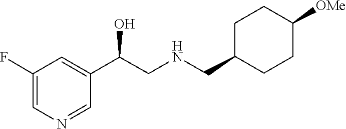 [SMILES: CO[C@H]1CC[C@@H](CNC[C@H](O)c2cncc(F)c2)CC1] | (R)-1-(5-fluoro-3- pyridyl)-2-({[(1s,4S)-4- methoxycyclohexyl]meth- yl}amino)-1-ethanol | ++ | − |
| 247 | 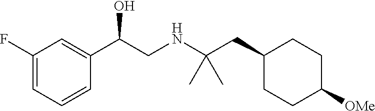 [SMILES: CO[C@H]1CC[C@@H](CC(C)(C)NC[C@H](O)c2cccc(F)c2)CC1] | (R)-2-{1,1-dimethyl-2- [(1s,4S)-4- methoxycyclohexyl]ethyl- amino}-1-(m- fluorophenyl)-1-ethanol | +++ | − |
| 248 | 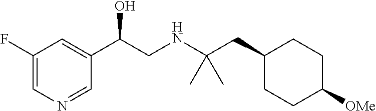 [SMILES: CO[C@H]1CC[C@@H](CC(C)(C)NC[C@H](O)c2cncc(F)c2)CC1] | (R)-2-{1,1-dimethyl-2- [(1s,4S)-4- methoxycyclohexyl]ethyl- amino}-1-(5-fluoro-3- pyridyl)-1-ethanol | +++ | − |
| 249 | 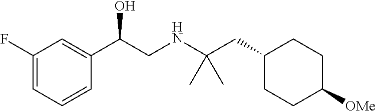 [SMILES: CO[C@H]1CC[C@H](CC(C)(C)NC[C@H](O)c2cccc(F)c2)CC1] | (R)-2-{1,1-dimethyl-2- [(1r,4R)-4- methoxycyclohexyl]ethyl- amino}-1-(m- fluorophenyl)-1-ethanol | +++ | − |
| 250 | 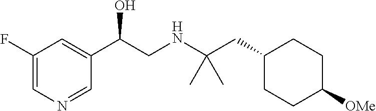 [SMILES: CO[C@H]1CC[C@H](CC(C)(C)NC[C@H](O)c2cncc(F)c2)CC1] | (R)-2-{1,1-dimethyl-2- [(1r,4R)-4- methoxycyclohexyl]ethyl- amino}-1-(5-fluoro-3- pyridyl)-1-ethanol | ++ | − |
| 251 | 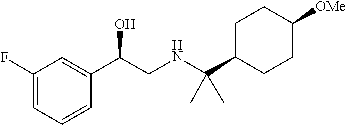 [SMILES: CO[C@H]1CC[C@@H](C(C)(C)NC[C@H](O)c2cccc(F)c2)CC1] | (R)-1-(m-fluroophenyl)- 2-{1-methyl-1- [(1s,4S)-4- methoxycyclohexyl]ethyl- amino}-1-ethanol | − | − |
| 252 | 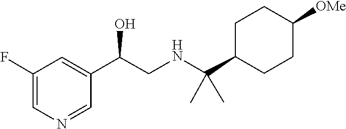 [SMILES: CO[C@H]1CC[C@@H](C(C)(C)NC[C@H](O)c2cncc(F)c2)CC1] | (R)-1-(5-fluoro-3- pyridyl)-2-{1-methyl-1- [(1s,4S)-4- methoxycyclohexyl]ethyl- amino}-1-ethanol | − | − |
| 253 | 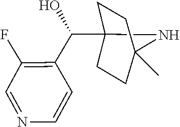 [SMILES: CC12CCC([C@@H](O)c3ccncc3F)(CC1)N2] | (S)-(3-fluoro-4- pyridyl)(4-methyl-7- azabicyclo[2.2.1]hept- 1-yl)methanol | − | − |
| 254 | 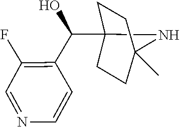 [SMILES: CC12CCC([C@H](O)c3ccncc3F)(CC1)N2] | (R)-(3-fluoro-4- pyridyl)(4-methyl-7- azabicyclo[2.2.1]hept- 1-yl)methanol | − | − |
| 255 | 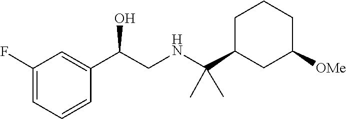 [SMILES: CO[C@@H]1CCC[C@H](C(C)(C)NC[C@H](O)c2cccc(F)c2)C1] | (R)-2-{1-[(1S,3R)-3- methoxycyclohexyl]-1- methylethylamino}-1- (m-fluorophenyl)-1- ethanol | +++ | − |
| 256 | 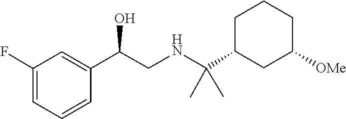 [SMILES: CO[C@H]1CCC[C@@H](C(C)(C)NC[C@H](O)c2cccc(F)c2)C1] | (R)-2-{1-[(1R,3S)-3- methoxycyclohexyl]-1- methylethylamino)}-1- (m-fluorophenyl)-1- ethanol | +++ | + |
| 257 | 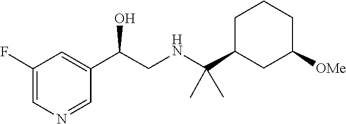 [SMILES: CO[C@@H]1CCC[C@H](C(C)(C)NC[C@H](O)c2cncc(F)c2)C1] | (R)-2-{1-[(1S,3R)-3- methoxycyclohexyl]-1- methylethylamino}-1- (5-fluoro-3-pyridyl)-1- ethanol | ++ | − |
| 258 | 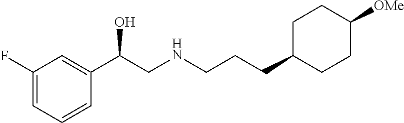 [SMILES: CO[C@H]1CC[C@@H](CCCNC[C@H](O)c2cccc(F)c2)CC1] | (R)-1-(m-fluorophenyl)- 2-{3-[(1s,4R)-4- methoxycyclohexyl]propyl- amino}-1-ethanol | +++ | ++ |
| 259 | 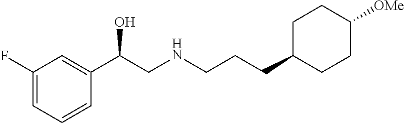 [SMILES: CO[C@H]1CC[C@H](CCCNC[C@H](O)c2cccc(F)c2)CC1] | (R)-1-(m-fluorophenyl)- 2-{3-[(1r,4S)-4- methoxycyclohexyl]propyl- amino}-1-ethanol | + | − |
| 260 | 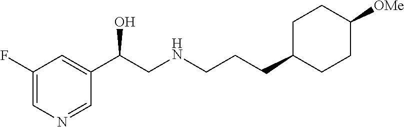 [SMILES: CO[C@H]1CC[C@@H](CCCNC[C@H](O)c2cncc(F)c2)CC1] | (R)-1-(5-fluoro-3- pyridyl)-2-{3-[(1s,4R)- 4- methoxycyclohexyl]propyl- amino}-1-ethanol | +++ | − |
| 261 | 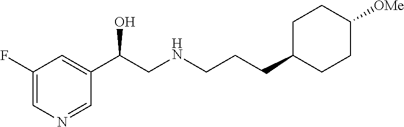 [SMILES: CO[C@H]1CC[C@H](CCCNC[C@H](O)c2cncc(F)c2)CC1] | (R)-1-(5-fluoro-3- pyridyl)-2-{3-[(1r,4S)- 4- methoxycyclohexyl]propyl- amino}-1-ethanol | − | − |
| 262 | 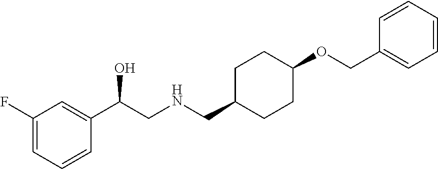 [SMILES: O[C@@H](CNC[C@H]1CC[C@@H](OCc2ccccc2)CC1)c1cccc(F)c1] | (R)-1-(m-fluorophenyl)- 2-({[(1s,4S)-4- (benzyloxy)cyclohexyl] methyl}amino)-1- ethanol | + | − |
| 263 | 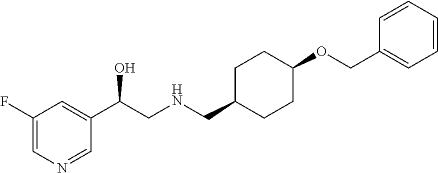 [SMILES: O[C@@H](CNC[C@H]1CC[C@@H](OCc2ccccc2)CC1)c1cncc(F)c1] | (R)-1-(5-fluoro-3- pyridyl)-2-({[(1s,4S)-4- (benzyloxy)cyclohexyl] methyl}amino)-1- ethanol | +++ | − |
| 264 | 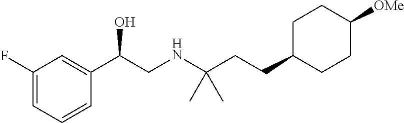 [SMILES: CO[C@H]1CC[C@@H](CCC(C)(C)NC[C@H](O)c2cccc(F)c2)CC1] | (R)-2-{1,1-dimethyl-3- [(1s,R)-4- methoxycyclohexyl]propyl- amino}-1-(m- fluorophenyl)-1-ethanol | +++ | + |
| 265 | 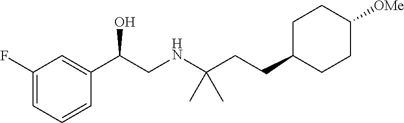 [SMILES: CO[C@H]1CC[C@H](CCC(C)(C)NC[C@H](O)c2cccc(F)c2)CC1] | (R)-2-{1,1-dimethyl-3- [(1r,4S)-4- methoxycyclohexyl]propyl- amino}-1-(m- fluorophenyl)-1-ethanol | ++ | − |
| 266 | 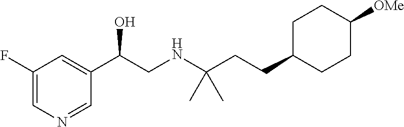 [SMILES: CO[C@H]1CC[C@@H](CCC(C)(C)NC[C@H](O)c2cncc(F)c2)CC1] | (1s,4R)-4- methoxycyclohexyl]propyl- amino}-1-(5-fluoro-3- pyridyl)-1-ethanol | +++ | − |
| 267 | 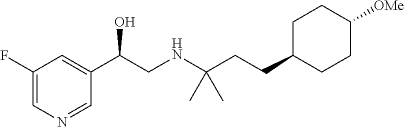 [SMILES: CO[C@H]1CC[C@H](CCC(C)(C)NC[C@H](O)c2cncc(F)c2)CC1] | (R)-2-{1,1-dimethyl-3- [(1r,4S)-4- methoxycyclohexyl]propyl- amino}-1-(5-fluoro-3- pyrdiyl)-1-ethanol | + | − |
| 268 | 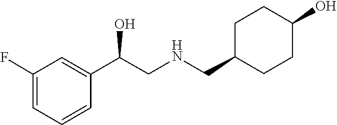 [SMILES: O[C@@H](CNC[C@H]1CC[C@@H](O)CC1)c1cccc(F)c1] | (R)-1-(m-fluorophenyl)- 2-({[(1s,4S)-4- hydroxycyclohexyl]meth- yl}amino)-1-ethanol | + | − |
| 269 | 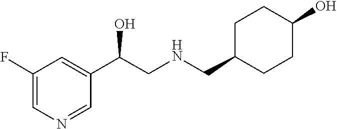 [SMILES: O[C@@H](CNC[C@H]1CC[C@@H](O)CC1)c1cncc(F)c1] | (R)-1-(5-fluoro-3- pyridyl)-2-({[(1s,4S)-4- hydroxycyclohexyl]meth- yl}amno)-1-ethanol | + | − |
| 270 | 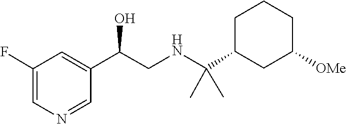 [SMILES: CO[C@H]1CCC[C@@H](C(C)(C)NC[C@H](O)c2cncc(F)c2)C1] | (R)-2-{1-[(1R,3S)-3- methoxycyclohexyl]-1- methylethylamino}-1- (5-fluoro-3-pyridyl)-1- ethanol | +++ | ++ |
| 271 | 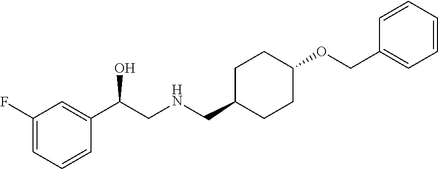 [SMILES: O[C@@H](CNC[C@H]1CC[C@H](OCc2ccccc2)CC1)c1cccc(F)c1] | (R)-1-(m-fluorophenyl)- 2-({[(1r,4R)-4- (benzyloxy)cyclohexyl] methyl}amino)-1- ethanol | + | − |
| 272 | 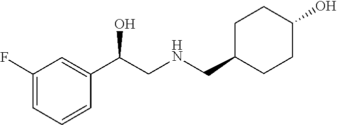 [SMILES: O[C@@H](CNC[C@H]1CC[C@H](O)CC1)c1cccc(F)c1] | (R)-1-(m-fluorophenyl)- 2-({[(1r,4R)-4- hydroxycycloehxyl]meth- yl}amino)-1-ethanol | + | − |
| 273 | 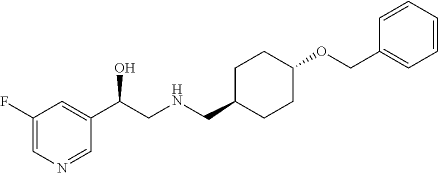 [SMILES: O[C@@H](CNC[C@H]1CC[C@H](OCc2ccccc2)CC1)c1cncc(F)c1] | (R)-1-(5-fluoro-3- pyrdiyl)-2-({[(1r,4R)-4- (benzyloxy)cyclohexyl] methyl}amino)-1- ethanol | + | − |
| 274 | 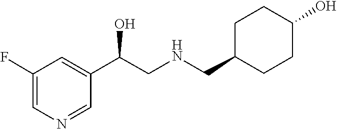 [SMILES: O[C@@H](CNC[C@H]1CC[C@H](O)CC1)c1cncc(F)c1] | (R)-1-(5-fluoro-3- pyridyl)-2-({[(1r,4R)-4- hydroxycyclohexyl]meth- yl}amino)-1-ethanol | − | − |
| 275 | 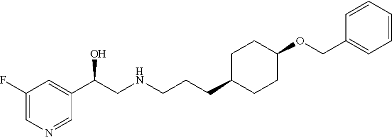 [SMILES: O[C@@H](CNCCC[C@H]1CC[C@@H](OCc2ccccc2)CC1)c1cncc(F)c1] | (R)-1-(5-fluoro-3- pyridyl)-2-{3-[(1s,4R)- 4- (benzyloxy)cyclohexyl]pro- pylamino}-1-ethanol | ++ | − |
| 276 | 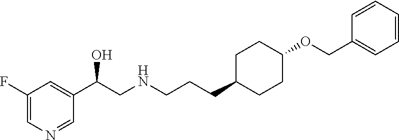 [SMILES: O[C@@H](CNCCC[C@H]1CC[C@H](OCc2ccccc2)CC1)c1cncc(F)c1] | (R)-1-(5-fluoro-3- pyridyl)-2-{3-[(1r,4S)- 4- (benzyloxy)cyclohexyl]pro- pylamino}-1-ethanol | + | − |
| 277 | 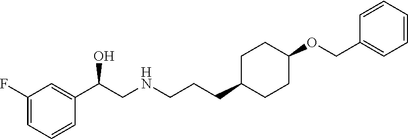 [SMILES: O[C@@H](CNCCC[C@H]1CC[C@@H](OCc2ccccc2)CC1)c1cccc(F)c1] | (R)-1-(m-fluorophenyl)- 2-{3-[(1s,4R)-4- (benzyloxy)cyclohexyl]pro- pylamino}-1-ethanol | + | − |
| 278 | 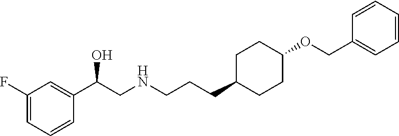 [SMILES: O[C@@H](CNCCC[C@H]1CC[C@H](OCc2ccccc2)CC1)c1cccc(F)c1] | (R)-1-(m-fluoropheny)- 2-{3-[(1r,4S)-4- (benzyloxy)cyclohexyl]pro- pylamino}-1-ethanol | + | − |
| 279 | 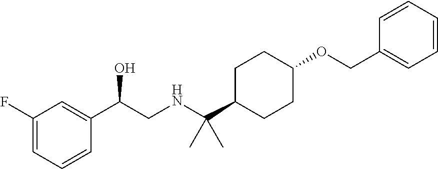 [SMILES: CC(C)(NC[C@H](O)c1cccc(F)c1)[C@H]1CC[C@H](OCc2ccccc2)CC1] | (R)-1-(m-fluorophenyl)- 2-{1-methyl-1- [(1r,4R)-4- (benzyloxy)cyclohexyl]eth- ylamino}-1-ethanol | ++ | − |
| 280 | 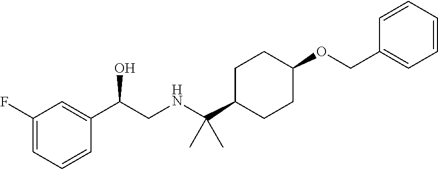 [SMILES: CC(C)(NC[C@H](O)c1cccc(F)c1)[C@H]1CC[C@@H](OCc2ccccc2)CC1] | (R)-1-(m-fluorophenyl)- 2-{1-methyl-1- [(1s,4S)-4- (benzyloxy)cyclohexyl]eth- ylamino}-1-ethanol | +++ | − |
| 281 | 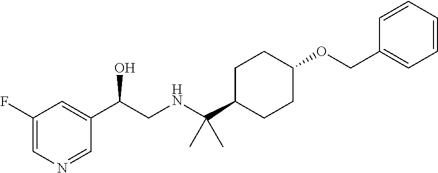 [SMILES: CC(C)(NC[C@H](O)c1cncc(F)c1)[C@H]1CC[C@H](OCc2ccccc2)CC1] | (R)-1-(5-fluoro-3- pyridyl)-2-{1-methyl-1- [(1r,4R)-4- (benzyloxy)cyclohexyl]eth- ylamino}-1-ethanol | ++ | − |
| 282 | 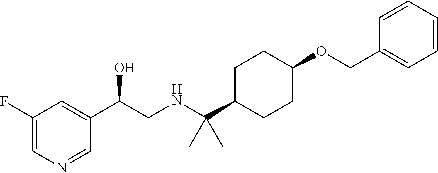 [SMILES: CC(C)(NC[C@H](O)c1cncc(F)c1)[C@H]1CC[C@@H](OCc2ccccc2)CC1] | (R)-1-(5-fluoro-3- pyridyl)-2-{1-methyl-1- [(1s,4S)-4- (benzyloxy)cylcohexyl]eth- ylamino}-1-ethanol | ++ | − |
| 283 | 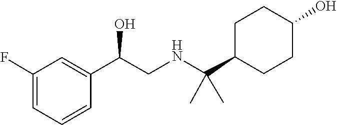 [SMILES: CC(C)(NC[C@H](O)c1cccc(F)c1)[C@H]1CC[C@H](O)CC1] | (R)-1-(m-fluorophenyl)- 2-{1-methyl-1- [(1r,4R)-4- hydroxycyclohexyl]ethyl- amino}-1-ethanol | ++ | − |
| 284 | 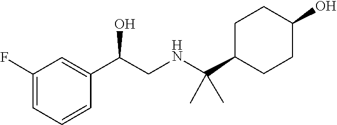 [SMILES: CC(C)(NC[C@H](O)c1cccc(F)c1)[C@H]1CC[C@@H](O)CC1] | (R)-1-(m-fluorophenyl)- 2-{1-methyl-1- [(1s,4S)-4- hydroxycyclohexyl]ethyl- amino}-1-ethanol | ++ | − |
| 285 | 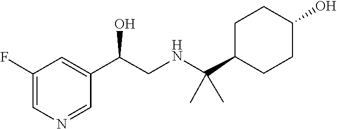 [SMILES: CC(C)(NC[C@H](O)c1cncc(F)c1)[C@H]1CC[C@H](O)CC1] | (R)-1-(5-fluoro-3- pyridyl)-2-{1-methyl-1- [(1r,4R)-4- hydroxycyclohexyl]ethyl- amino}-1-ethanol | ++ | − |
| 286 | 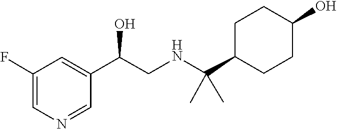 [SMILES: CC(C)(NC[C@H](O)c1cncc(F)c1)[C@H]1CC[C@@H](O)CC1] | (R)-1-(5-fluoro-3- pyridyl)-2-{1-methyl-1- [(1s,4S)-4- hydroxycyclohexyl]ethyl- amino}-1-ethanol | + | − |
| 287 | 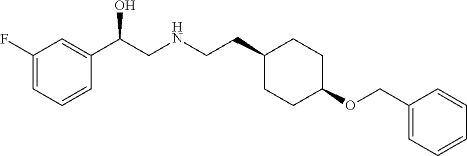 [SMILES: O[C@@H](CNCC[C@H]1CC[C@@H](OCc2ccccc2)CC1)c1cccc(F)c1] | (R)-1-(m-fluorophenyl)- 2-{2-[(1s,4S)-4- (benzyloxy)cyclohexyl]eth- ylamino}-1-ethanol | ++ | − |
| 288 | 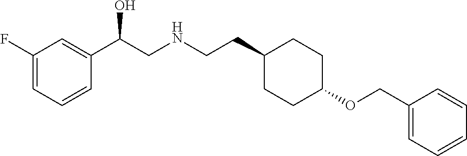 [SMILES: O[C@@H](CNCC[C@H]1CC[C@H](OCc2ccccc2)CC1)c1cccc(F)c1] | (R)-1-(m-fluorophenyl)- 2-{2-[(1r,4R)-4- (benzyloxy)cyclohexyl]eth- ylamino}-1-ethanol | + | − |
| 289 | 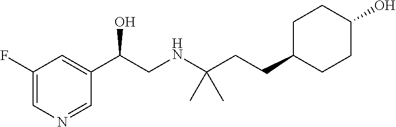 [SMILES: CC(C)(CC[C@H]1CC[C@H](O)CC1)NC[C@H](O)c1cncc(F)c1] | (R)-2-{1,1-dimethyl-3- [(1r,4S)-4- hydroxycyclohexyl]propyl- amino}-1-(5-fluoro-3- pyridyl)-1-ethanol | + | − |
| 290 | 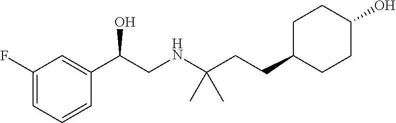 [SMILES: CC(C)(CC[C@H]1CC[C@H](O)CC1)NC[C@H](O)c1cccc(F)c1] | (R)-2-{1,1-dimethyl-3- [(1r,4S)-4- hydroxycyclohexyl]propyl- amino}-1-(m- fluorophenyl)-1-ethanol | + | − |
| 291 | 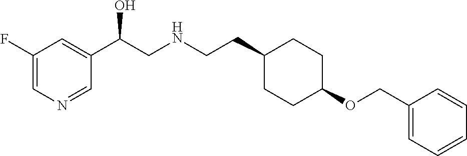 [SMILES: O[C@@H](CNCC[C@H]1CC[C@@H](OCc2ccccc2)CC1)c1cncc(F)c1] | (R)-1-(5-fluoro-3- pyridyl)-2-{2-[(1s,4S)- 4- (benzyloxy)cyclohexyl]eth- ylamino}-1-ethanol | ++ | − |
| 292 | 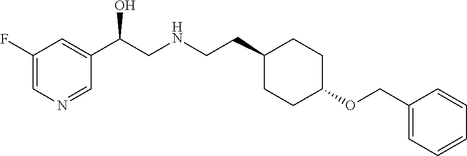 [SMILES: O[C@@H](CNCC[C@H]1CC[C@H](OCc2ccccc2)CC1)c1cncc(F)c1] | (R)-1-(5-fluoro-3- pyridyl)-2-{2-[(1r,4R)- 4- (benzyloxy)cyclohexyl]eth- ylamino}-1-ethanol | − | − |
| 293 | 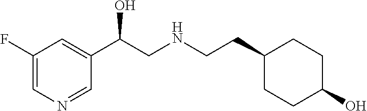 [SMILES: O[C@@H](CNCC[C@H]1CC[C@@H](O)CC1)c1cncc(F)c1] | (R)-1-(5-fluoro-3- pyridyl)-2-{2-[(1s,4S)- 4- hydroxycyclohexyl]ethyl- amino}-1-ethanol | + | − |
| 294 | 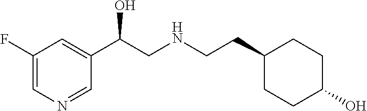 [SMILES: O[C@@H](CNCC[C@H]1CC[C@H](O)CC1)c1cncc(F)c1] | (R)-1-(5-fluoro-3- pyridyl)-2-{2-[(1r,4R)- 4- hydroxycyclohexyl]ethyl- amino}-1-ethanol | + | − |
| 295 | 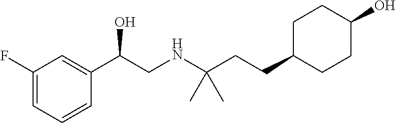 [SMILES: CC(C)(CC[C@H]1CC[C@@H](O)CC1)NC[C@H](O)c1cccc(F)c1] | (R)-2-{1,1-dimethyl-3- [(1s,4R)-4- hydroxycyclohexyl]propyl- amino}-1-(m- fluorophenyl)-1-ethanol | +++ | − |
| 296 | 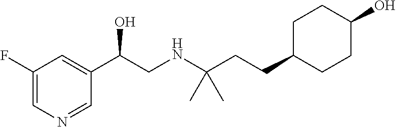 [SMILES: CC(C)(CC[C@H]1CC[C@@H](O)CC1)NC[C@H](O)c1cncc(F)c1] | (R)-2-{1,1-dimethyl-3- [(1s,4R)-4- hydroxycyclohexyl]propyl- amino}-1-(5-fluoro-3- pyridyl)-1-ethanol | +++ | − |
| 297 | 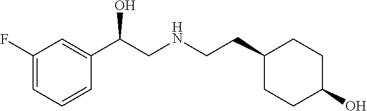 [SMILES: O[C@@H](CNCC[C@H]1CC[C@@H](O)CC1)c1cccc(F)c1] | (R)-1-(m-fluorophenyl)- 2-{2-[(1s,4S)-4- hydroxycyclohexyl]ethyl- amino}-1-ethanol | ++ | − |
| 298 | 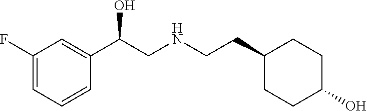 [SMILES: O[C@@H](CNCC[C@H]1CC[C@H](O)CC1)c1cccc(F)c1] | (R)-1-(m-fluorophenyl)- 2-{2-[(1r,4R)-4- hydroxycyclohexyl]ethyl- amino}-1-ethanol | + | − |
| 299 | 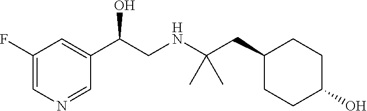 [SMILES: CC(C)(C[C@H]1CC[C@H](O)CC1)NC[C@H](O)c1cncc(F)c1] | (R)-2-{1,1-dimethyl-2- [(1r,4R)-4- hydroxycyclohexyl]ethyl- amino}-1-(5-fluoro-3- pyridyl)-1-ethanol | ++ | − |
| 300 | 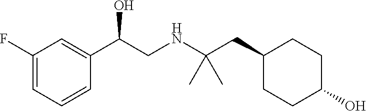 [SMILES: CC(C)(C[C@H]1CC[C@H](O)CC1)NC[C@H](O)c1cccc(F)c1] | (R)-2-{1,1-dimethyl-2- [(1r,4R)-4- hydroxycyclohexyl]ethyl- amino}-1-(m- fluorophenyl)-1-ethanol | +++ | − |
| 301 | 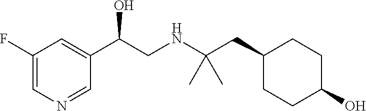 [SMILES: CC(C)(C[C@H]1CC[C@@H](O)CC1)NC[C@H](O)c1cncc(F)c1] | (R)-2-{1,1-dimethyl-2- [(1s,4S)-4- hydroxycyclohexyl]ethyl- amino}-1-(5-fluoro-3- pyridyl)-1-ethanol | +++ | − |
| 302 | 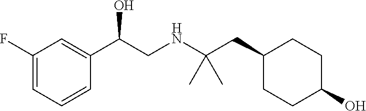 [SMILES: CC(C)(C[C@H]1CC[C@@H](O)CC1)NC[C@H](O)c1cccc(F)c1] | (R)-2-{1,1-dimethyl-2- [(1s,4S)-4- hydroxycyclohexyl]ethyl- amino}-1-(m- fluorophenyl)-1-ethanol | ++ | − |
| 303 | 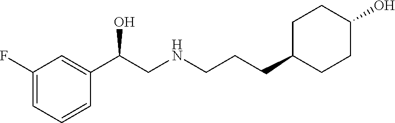 [SMILES: O[C@@H](CNCCC[C@H]1CC[C@H](O)CC1)c1cccc(F)c1] | (R)-1-(m-fluorophenyl)- 2-{3-[(1r,4S)-4- hydroxycyclohexyl]propyl- amino}-1-ethanol | ++ | − |
| 304 | 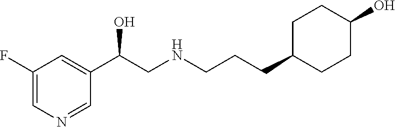 [SMILES: O[C@@H](CNCCC[C@H]1CC[C@@H](O)CC1)c1cncc(F)c1] | (R)-1-(5-fluoro-3- pyridyl)-2-{3-[(1s,4R)- 4- hydroxycyclohexyl]propyl- amino}-1-ethanol | ++ | − |
| 305 | 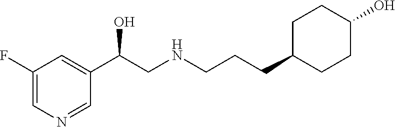 [SMILES: O[C@@H](CNCCC[C@H]1CC[C@H](O)CC1)c1cncc(F)c1] | (R)-1-(5-fluoro-3- pyridyl)-2-{3-[(1r,4S)- 4- hydroxycyclohexyl]propyl- amino}-1-ethanol | + | − |
| 306 | 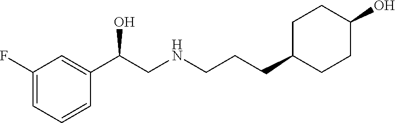 [SMILES: O[C@@H](CNCCC[C@H]1CC[C@@H](O)CC1)c1cccc(F)c1] | (R)-1-(m-fluorophenyl)- 2-{3-[(1s,4R)-4- hydroxycyclohexyl]propyl- amino}-1-ethanol | + | − |
| 307 | 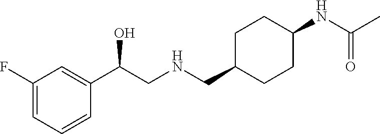 [SMILES: CC(=O)N[C@H]1CC[C@@H](CNC[C@H](O)c2cccc(F)c2)CC1] | N-[(1S,4s)-4-{[(R)-2- (m-fluorophenyl)-2- hydroxyethylamino]meth- yl}cyclohexyl]acetamide | ++ | − |
| 308 | 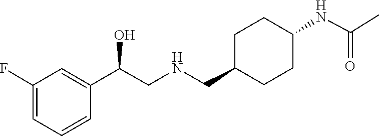 [SMILES: CC(=O)N[C@H]1CC[C@H](CNC[C@H](O)c2cccc(F)c2)CC1] | N-[(1R,4r)-4-{[(R)-2- (m-fluorophenyl)-2- hydroxyethylamino]meth- yl}cyclohexyl]acetamide | ++ | − |
| 309 | 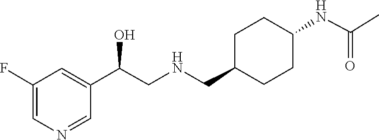 [SMILES: CC(=O)N[C@H]1CC[C@H](CNC[C@H](O)c2cncc(F)c2)CC1] | N-[(1R,4r)-4-{[(R)-2- (5-fluoro-3-pyridyl)-2- hydroxyethylamino]meth- yl}cyclohexyl]acetamide | ++ | − |
| 310 | 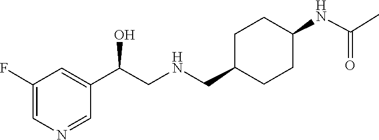 [SMILES: CC(=O)N[C@H]1CC[C@@H](CNC[C@H](O)c2cncc(F)c2)CC1] | N-[(1S,4s)-4-{[(R)-2- (5-fluoro-3-pyridyl)-2- hydroxyethylamino]meth- yl}cyclohexyl]acetamide | + | − |
| 311 | 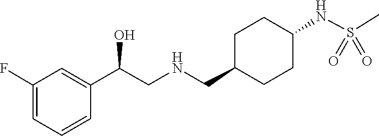 [SMILES: CS(=O)(=O)N[C@H]1CC[C@H](CNC[C@H](O)c2cccc(F)c2)CC1] | (R)-1-(m-fluorophenyl)- 2-({[(1r,4R)-4- (mesylamino)cyclohexyl] methyl}amino)-1- ethanol | ++ | − |
| 312 | 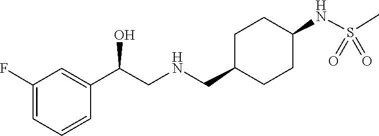 [SMILES: CS(=O)(=O)N[C@H]1CC[C@@H](CNC[C@H](O)c2cccc(F)c2)CC1] | (R)-1-(m-fluorophenyl)- 2-({[(1s,4S)-4- (mesylamino)cyclohexyl] methyl}amino)-1- ethanol | ++ | − |
| 313 | 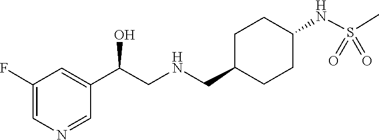 [SMILES: CS(=O)(=O)N[C@H]1CC[C@H](CNC[C@H](O)c2cncc(F)c2)CC1] | (R)-1-(5-fluoro-3- pyridyl)-2-({[(1r,4R)-4- (mesylamino)cyclohexyl] methyl}amino)-1- ethanol | + | − |
| 314 | 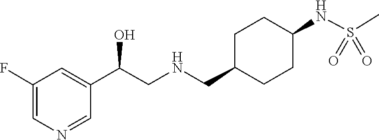 [SMILES: CS(=O)(=O)N[C@H]1CC[C@@H](CNC[C@H](O)c2cncc(F)c2)CC1] | (R)-1-(5-fluoro-3- pyridyl)-2-({[(1s,4S)-4- (mesylamino)cyclohexyl] methyl}amino)-1- ethanol | ++ | − |
| 315 | 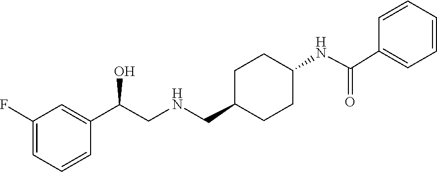 [SMILES: O=C(N[C@H]1CC[C@H](CNC[C@H](O)c2cccc(F)c2)CC1)c1ccccc1] | N-[(1R,4r)-4-{[(R)-2- (m-fluorophenyl)-2- hydroxyethylamino]meth- yl}cyclohexyl]benzamide | +++ | − |
| 316 | 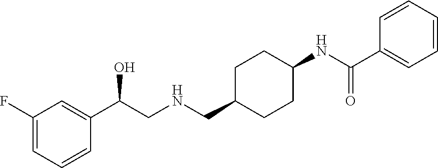 [SMILES: O=C(N[C@H]1CC[C@@H](CNC[C@H](O)c2cccc(F)c2)CC1)c1ccccc1] | N-[(1S,4s)-4-{[(R)-2- (m-fluorophenyl)-2- hydroxyethylamino]meth- yl}cyclohexyl]benzamide | ++ | − |
| 317 | 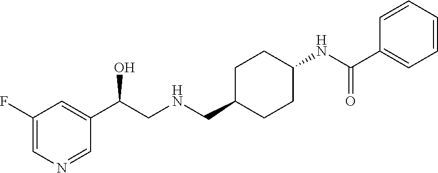 [SMILES: O=C(N[C@H]1CC[C@H](CNC[C@H](O)c2cncc(F)c2)CC1)c1ccccc1] | N-[(1R,4r)-4-{[(R)-2- (5-fluoro-3-pyridyl)-2- hydroxyethylamino]meth- yl}cyclohexyl]benzamide | ++ | − |
| 318 | 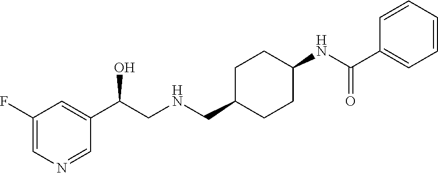 [SMILES: O=C(N[C@H]1CC[C@@H](CNC[C@H](O)c2cncc(F)c2)CC1)c1ccccc1] | N-[(1S,4s)-4-{[(R)-2- (5-fluoro-3-pyridyl)-2- hydroxyethylamino]meth- yl}cyclohexyl]benzamide | ++ | − |
| 319 | 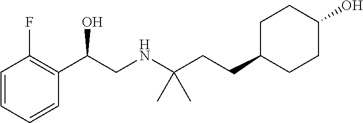 [SMILES: CC(C)(CC[C@H]1CC[C@H](O)CC1)NC[C@H](O)c1ccccc1F] | (R)-2-{1,1-dimethyl-3- [(1r,4S)-4- hydroxycyclohexyl]propyl- amino}-1-(o- fluorophenyl)-1-ethanol | +++ | − |
| 320 | 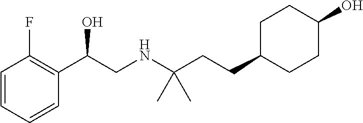 [SMILES: CC(C)(CC[C@H]1CC[C@@H](O)CC1)NC[C@H](O)c1ccccc1F] | (R)-2-{1,1-dimethyl-3- [(1s,4R)-4- hydroxycyclohexyl]propyl- amino}-1-(o- fluorophenyl)-1-ethanol | +++ | + |
| 321 | 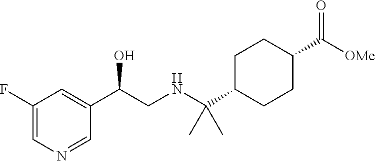 [SMILES: COC(=O)[C@H]1CC[C@@H](C(C)(C)NC[C@H](O)c2cncc(F)c2)CC1] | (1S,4s)-8-[(R)-2-(5- fluoro-3-pyridyl)-2- hydroxyethylamino]-7- methoxy-p-menthan-7- one | ++ | − |
| 322 | 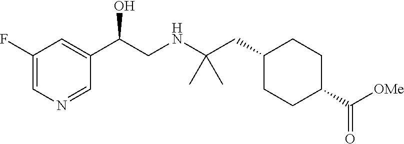 [SMILES: COC(=O)[C@H]1CC[C@@H](CC(C)(C)NC[C@H](O)c2cncc(F)c2)CC1] | methyl (1S,4s)-4-{2- [(R)-2-(5-fluoro-3- pyridyl)-2- hydroxyethylamino]-2- methylpropyl}cyclohexane- carboxylate | +++ | − |
| 323 | 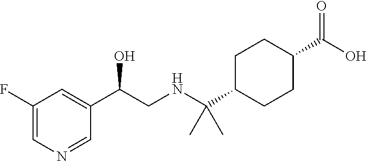 [SMILES: CC(C)(NC[C@H](O)c1cncc(F)c1)[C@H]1CC[C@@H](C(=O)O)CC1] | (1S,4s)-8-[(R)-2-(5- fluoro-3-pyridyl)-2- hydroxyethylamino]-7- hydroxy-p-menthan-7- one | + | ++ |
| 324 | 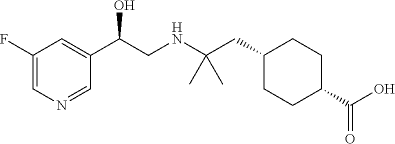 [SMILES: CC(C)(C[C@H]1CC[C@@H](C(=O)O)CC1)NC[C@H](O)c1cncc(F)c1] | (1S,4s)-4-{2-[(R)-2-(5- fluoro-3-pyridyl)-2- hydroxyethylamino]-2- methylpropyl}cyclohexane- carboxylic acid | + | − |
| 325 | 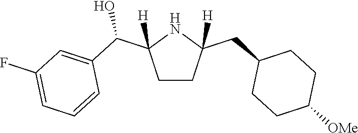 [SMILES: CO[C@H]1CC[C@H](C[C@@H]2CC[C@H]([C@@H](O)c3cccc(F)c3)N2)CC1] | (S)-[(2R,5S)-5- {[(1r,4S)-4- methoxycyclohexyl]meth- yl}-2-pyrrolidinyl](m- fluorophenyl)methanol | +++ | − |
| 326 | 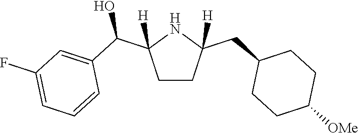 [SMILES: CO[C@H]1CC[C@H](C[C@@H]2CC[C@H]([C@H](O)c3cccc(F)c3)N2)CC1] | (R)-[(2R,5S)-5- {[(1r,4S)-4- methoxycyclohexyl]meth- yl}-2-pyrrolidinyl](m- fluorophenyl)methanol | +++ | − |
| 327 | 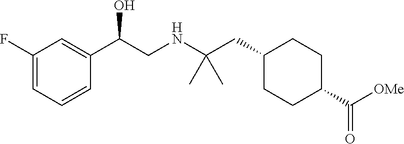 [SMILES: COC(=O)[C@H]1CC[C@@H](CC(C)(C)NC[C@H](O)c2cccc(F)c2)CC1] | methyl (1S,4s)-4-{2- [(R)-2-(m- fluorophenyl)-2- hydroxyethylamino]-2- methylpropyl}cyclohexane- carboxylate | +++ | − |
| 328 | 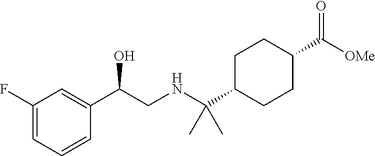 [SMILES: COC(=O)[C@H]1CC[C@@H](C(C)(C)NC[C@H](O)c2cccc(F)c2)CC1] | (1S,4s)-8-[(R)-2-(m- fluorophenyl)-2- hydroxyethylamino]-7- methoxy-p-menthan-7- one | +++ | − |
| 329 | 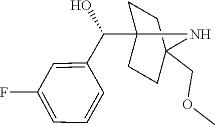 [SMILES: COCC12CCC([C@@H](O)c3cccc(F)c3)(CC1)N2] | (S)-(m-fluorophenyl){4- (methoxymethyl)-7- azabicyclo[2.2.1]hept- 1-yl}methanol | + | − |
| 330 | 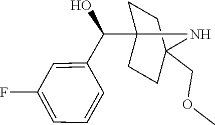 [SMILES: COCC12CCC([C@H](O)c3cccc(F)c3)(CC1)N2] | (R)-(m-fluorophenyl){4- (methoxymethyl)-7- azabicyclo[2.2.1]hept- 1-yl}methanol | +++ | − |
| 331 | 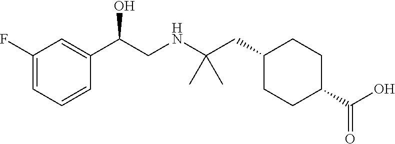 [SMILES: CC(C)(C[C@H]1CC[C@@H](C(=O)O)CC1)NC[C@H](O)c1cccc(F)c1] | (1S,4s)-4-{2-[(R)-2- (m-fluorophenyl)-2- hydroxyethylamino]-2- methylpropyl}cyclohexane- carboxylic acid | + | − |
| 332 | 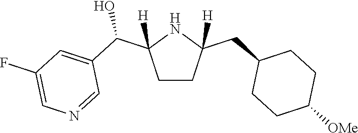 [SMILES: CO[C@H]1CC[C@H](C[C@@H]2CC[C@H]([C@@H](O)c3cncc(F)c3)N2)CC1] | (S)-[(2R,5S)-5- {[(1r,4S)-4- methoxycyclohexyl]meth- yl}-2-pyrrolidinyl](5- fluoro-3- pyridyl)methanol | + | − |
| 333 | 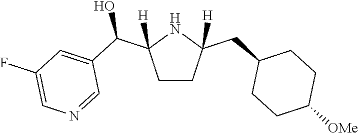 [SMILES: CO[C@H]1CC[C@H](C[C@@H]2CC[C@H]([C@H](O)c3cncc(F)c3)N2)CC1] | (R)-[(2R,5S)-5- {[(1r,4S)-4- methoxycyclohexyl]meth- yl}-2-pyrrolidinyl](5- fluoro-3- pyridyl)methanol | + | − |
| 334 | 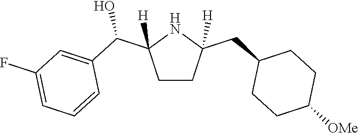 [SMILES: CO[C@H]1CC[C@H](C[C@H]2CC[C@H]([C@@H](O)c3cccc(F)c3)N2)CC1] | (S)-[(2R,5R)-5- {[(1r,4R)-4- methoxycyclohexyl]meth- yl}-2-pyrrolidinyl](m- fluorophenyl)methanol | + | − |
| 335 | 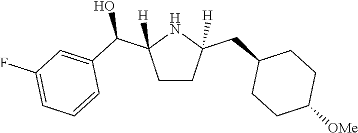 [SMILES: CO[C@H]1CC[C@H](C[C@H]2CC[C@H]([C@H](O)c3cccc(F)c3)N2)CC1] | (R)-[(2R,5R)-5- {[(1r,4R)-4- methoxycyclohexyl]meth- yl}-2-pyrrolidinyl](m- fluorophenyl)methanol | +++ | − |
| 336 | 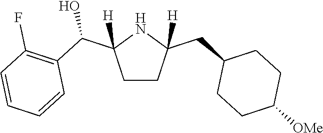 [SMILES: CO[C@H]1CC[C@H](C[C@@H]2CC[C@H]([C@@H](O)c3ccccc3F)N2)CC1] | (S)-[(2R,5S)-4- {[(1r,4S)-4- methoxycyclohexyl]meth- yl}-2-pyrrolidinyl](o- fluorophenyl)methanol | + | − |
| 337 | 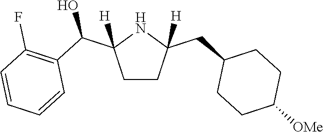 [SMILES: CO[C@H]1CC[C@H](C[C@@H]2CC[C@H]([C@H](O)c3ccccc3F)N2)CC1] | (R)-[(2R,5S)-5- {[(1r,4S)-4- methoxycyclohexyl]meth- yl}-2-pyrrolidinyl](o- fluorophenyl)methanol | +++ | − |
| 338 | 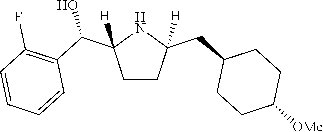 [SMILES: CO[C@H]1CC[C@H](C[C@H]2CC[C@H]([C@@H](O)c3ccccc3F)N2)CC1] | (S)-[(2R,5R)-5- {[(1r,4R)-4- methoxycyclohexyl]meth- yl}-2-pyrrolidinyl](o- fluorophenyl)methanol | ++ | − |
| 339 | 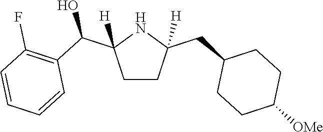 [SMILES: CO[C@H]1CC[C@H](C[C@H]2CC[C@H]([C@H](O)c3ccccc3F)N2)CC1] | (R)-[(2R,5R)-5- {[(1r,4R)-4- methoxycyclohexyl]meth- yl}-2-pyrrolidinyl](o- fluorophenyl)methanol | +++ | + |
| 340 | 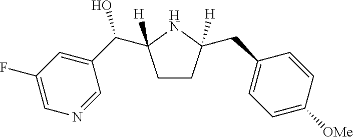 | (S)-[(2R,5R)-5- {[(1r,4R)-4- methoxycyclohexyl]meth- yl}-2-pyrrolidinyl](5- fluoro-3- pyridyl)methanol | + | − |
| 341 | 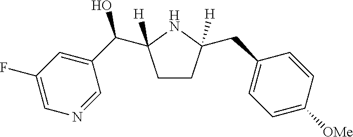 | (R)-[(2R,5R)-5- {[(1r,4R)-4- methoxycyclohexyl]meth- yl}-2-pyrrolidinyl](5- fluoro-3- pyridyl)methanol | +++ | − |
| 342 | 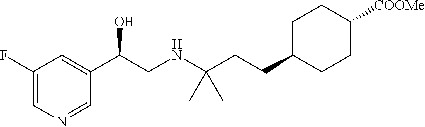 [SMILES: COC(=O)[C@H]1CC[C@H](CCC(C)(C)NC[C@H](O)c2cncc(F)c2)CC1] | methyl (1R,4r)-4-{3- [(R)-2-(5-fluoro-3- pyridyl)-2- hydroxyethylamino]-3- methylbutyl}cyclohexane- carboxylate | +++ | − |
| 343 | 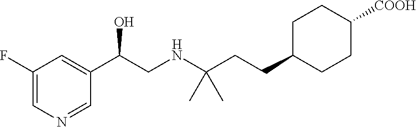 [SMILES: CC(C)(CC[C@H]1CC[C@H](C(=O)O)CC1)NC[C@H](O)c1cncc(F)c1] | (1R,4r)-4-{3-[(R)-2-(5- fluoro-3-pyridyl)-2- hydroxyethylamino]-3- methylbutyl}cyclohexane- carboxylic acid | + | − |
| 344 | 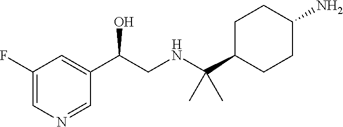 [SMILES: CC(C)(NC[C@H](O)c1cncc(F)c1)[C@H]1CC[C@H](N)CC1] | (R)-1-(5-fluoro-3- pyridyl)-2-{1-methyl-1- [(1r,4R)-4- aminocyclohexyl]ethyla- mino}-1-ethanol | − | − |
| 345 | 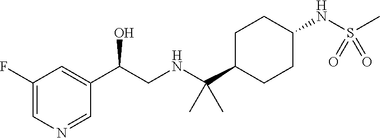 [SMILES: CC(C)(NC[C@H](O)c1cncc(F)c1)[C@H]1CC[C@H](NS(C)(=O)=O)CC1] | (R)-1-(5-fluoro-3- pyridyl)-2-{1-methyl-1- [(1r,4R)-4- (mesylamino)cyclohexyl] ethylamino}-1-ethanol | + | − |
| 346 | 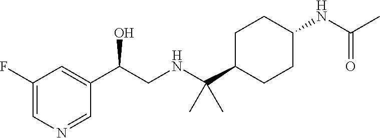 [SMILES: CC(=O)N[C@H]1CC[C@H](C(C)(C)NC[C@H](O)c2cncc(F)c2)CC1] | N-[(1R,4r)-4-{1-[(R)-2- (5-fluoro-3-pyridyl)-2- hydroxyethylamino]-1- methylethyl}cyclohexyl] acetamide | +++ | − |
| 347 | 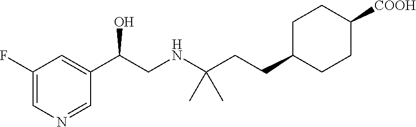 [SMILES: CC(C)(CC[C@H]1CC[C@@H](C(=O)O)CC1)NC[C@H](O)c1cncc(F)c1] | (1S,4s)-4-{3-[(R)-2-(5- fluoro-3-pyridyl)-2- hydroxyethylamino]-3- methylbutyl}cyclohexane- carboxylic acid | +++ | − |
| 348 | 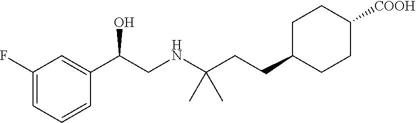 [SMILES: CC(C)(CC[C@H]1CC[C@H](C(=O)O)CC1)NC[C@H](O)c1cccc(F)c1] | (1R,4r)-4-{3-[(R)-2- (m-fluorophenyl)-2- hydroxyethylamino]-3- methylbutyl}cyclohexane- carboxylic acid | − | − |
| 349 | 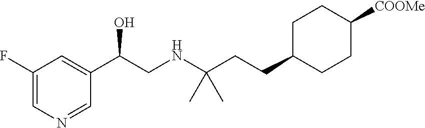 [SMILES: COC(=O)[C@H]1CC[C@@H](CCC(C)(C)NC[C@H](O)c2cncc(F)c2)CC1] | methyl (1S,4s)-4-{3- [(R)-2-(5-fluoro-3- pyridyl)-2- hydroxyethylamino]-3- methylbutyl}cyclohexane- carboxylate | +++ | + |
| 350 | 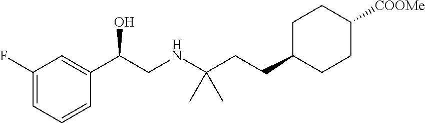 [SMILES: COC(=O)[C@H]1CC[C@H](CCC(C)(C)NC[C@H](O)c2cccc(F)c2)CC1] | methyl (1R,4r)-4-{3- [(R)-2-(m- fluorophenyl)-2- hydroxyethylamino]-3- methylbutyl}cyclohexane- carobxylate | +++ | − |
| 351 | 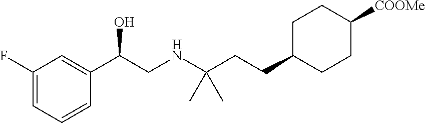 [SMILES: COC(=O)[C@H]1CC[C@@H](CCC(C)(C)NC[C@H](O)c2cccc(F)c2)CC1] | methyl (1S,4s)-4-{3- [(R)-2-(m- fluorophenyl)-2- hydroxyethylamino]-3- methylbutyl}cyclohexane- carboxylate | +++ | + |
| 352 | 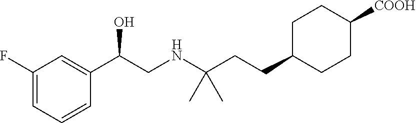 [SMILES: CC(C)(CC[C@H]1CC[C@@H](C(=O)O)CC1)NC[C@H](O)c1cccc(F)c1] | (1S,4s)-4-{3-[(R)-2- (m-fluorophenyl)-2- hydroxyethylamino]-3- methylbutyl}cyclohexane- carboxylc acid | + | − |
| 353 | 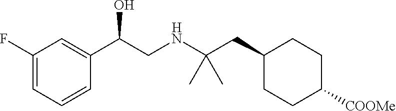 [SMILES: COC(=O)[C@H]1CC[C@H](CC(C)(C)NC[C@H](O)c2cccc(F)c2)CC1] | methyl (1R,4r)-4-{2- [(R)-2-(m- fluorophenyl)-2- hydroxyethylamino]-2- methylpropyl}cyclohexane- carboxylate | +++ | − |
| 354 | 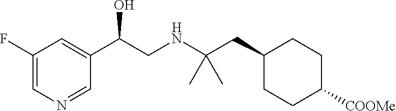 [SMILES: COC(=O)[C@H]1CC[C@H](CC(C)(C)NC[C@H](O)c2cncc(F)c2)CC1] | methyl (1R,4r)-4-{2- [(R)-2-(5-fluoro-3- pyridyl)-2- hydroxyethylamino]-2- methylpropyl}cyclohexane- carboxylate | ++ | − |
| 355 | 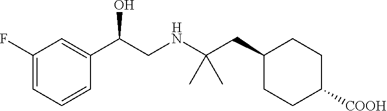 [SMILES: CC(C)(C[C@H]1CC[C@H](C(=O)O)CC1)NC[C@H](O)c1cccc(F)c1] | (1R,4r)-4-{2-[(R)-2- (m-fluorophenyl)-2- hydroxyethylamino]-2- methylpropyl}cyclohexane- carboxylic acid | ++ | − |
| 356 | 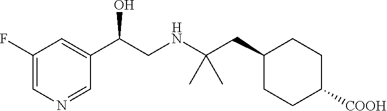 [SMILES: CC(C)(C[C@H]1CC[C@H](C(=O)O)CC1)NC[C@H](O)c1cncc(F)c1] | (1R,4r)-4-{2-[(R)-2-(5- fluoro-3-pyridyl)-2- hydroxyethylamino]-2- methylpropyl}cyclohexane- carboxylic acid | − | − |
| 357 | 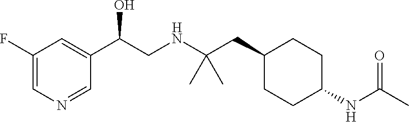 [SMILES: CC(=O)N[C@H]1CC[C@H](CC(C)(C)NC[C@H](O)c2cncc(F)c2)CC1] | N-[(1R,4r)-4-{2-[(R)-2- (5-fluoro-3-pyridyl)-2- hydroxyethylamino]-2- methylpropyl}cyclohexyl] acetamide | +++ | − |
| 358 | 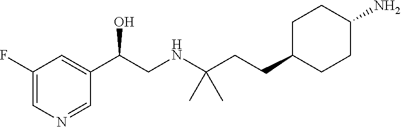 [SMILES: CC(C)(CC[C@H]1CC[C@H](N)CC1)NC[C@H](O)c1cncc(F)c1] | (R)-2-{1,1-dimethyl-3- [(1r,4R)-4- aminocyclohexyl]propyl- amino}-1-(5-fluoro-3- pyridyl)-1-ethanol | + | − |
| 359 | 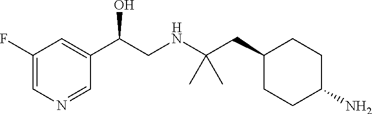 [SMILES: CC(C)(C[C@H]1CC[C@H](N)CC1)NC[C@H](O)c1cncc(F)c1] | (R)-2-{1,1-dimethyl-2- [(1r,4R)-4- aminocyclohexyl]ethyla- mino}-1-(5-fluoro-3- pyridyl)-1-ethanol | +++ | − |
| 360 | 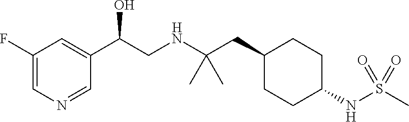 [SMILES: CC(C)(C[C@H]1CC[C@H](NS(C)(=O)=O)CC1)NC[C@H](O)c1cncc(F)c1] | (R)-2-{1,1-dimethyl-2- [(1r,4R)-4- (mesylamino)cyclohexyl] ethylamino}-1-(5- fluoro-3-pyridyl)-1- ethanol | +++ | − |
| 361 | 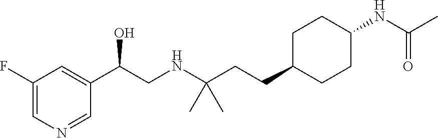 [SMILES: CC(=O)N[C@H]1CC[C@H](CCC(C)(C)NC[C@H](O)c2cncc(F)c2)CC1] | N-[(1R,4r)-4-{3-[(R)-2- (5-fluoro-3-pyridyl)-2- hydroxyethylamino]-3- methlbutyl}cyclohexyl] acetamide | +++ | − |
| 362 | 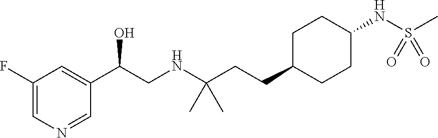 [SMILES: CC(C)(CC[C@H]1CC[C@H](NS(C)(=O)=O)CC1)NC[C@H](O)c1cncc(F)c1] | (R)-2-{1,1-dimethyl-3- [(1r,4R)-4- (mesylamino)cyclohexyl] propylamino}-1-(5- fluoro-3-pyridyl)-1- ethanol | ++ | − |
| 363 | 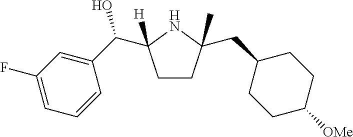 [SMILES: CO[C@H]1CC[C@H](C[C@]2(C)CC[C@H]([C@@H](O)c3cccc(F)c3)N2)CC1] | (S)-[(2R,5S)-5-methyl- 5-{[(1r,4S)-4- methoxycyclohexyl]meth- yl}-2-pyrrolidinyl](m- fluorophenyl)methanol | +++ | − |
| 364 | 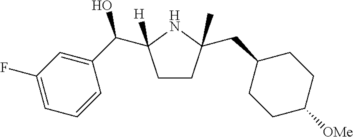 [SMILES: CO[C@H]1CC[C@H](C[C@]2(C)CC[C@H]([C@H](O)c3cccc(F)c3)N2)CC1] | (R)-[(2R,5S)-5-methyl- 5-{[(1r,4S)-4- methoxycyclohexyl]meth- yl}-2-pyrrolidinyl](m- fluorophenyl)methanol | +++ | ++ |
| 365 | 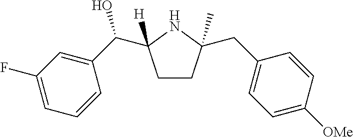 [SMILES: COc1ccc(C[C@@]2(C)CC[C@H]([C@@H](O)c3cccc(F)c3)N2)cc1] | (S)-{(2R,5R)-5-[(p- methoxyphenyl)methyl]- 5-methyl-2- pyrrolidinyl}(m- fluorophenyl)methanol | +++ | − |
| 366 | 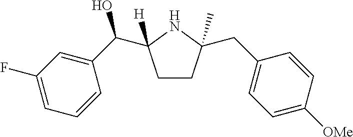 [SMILES: COc1ccc(C[C@@]2(C)CC[C@H]([C@H](O)c3cccc(F)c3)N2)cc1] | (R)-{(2R,5R)-5-[(p- methoxyphenyl)methyl]- 5-methyl-2- pyrrolidinyl}(m- fluorophenyl)methanol | +++ | + |
| 367 | 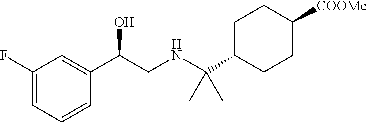 [SMILES: COC(=O)[C@H]1CC[C@H](C(C)(C)NC[C@H](O)c2cccc(F)c2)CC1] | (1R,4r)-8-[(R)-2-(m- fluorophenyl)-2- hydroxyethylamino]-7- methoxy-p-menthan-7- one | +++ | + |
| 368 | 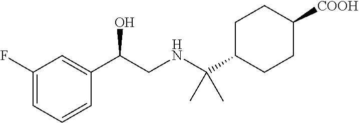 [SMILES: CC(C)(NC[C@H](O)c1cccc(F)c1)[C@H]1CC[C@H](C(=O)O)CC1] | (1R,4r)-8-[(R)-2-(m- fluorophenyl)-2- hydroxyethylamino]-7- hydroxy-p-menthan-7- one | + | − |
| 369 | 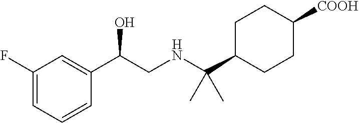 [SMILES: CC(C)(NC[C@H](O)c1cccc(F)c1)[C@H]1CC[C@@H](C(=O)O)CC1] | (1S,4s)-8-[(R)-2-(m- fluorophenyl)-2- hydroxyethylamino]-7- hydroxy-p-menthan-7- one | ++ | − |
| 370 | 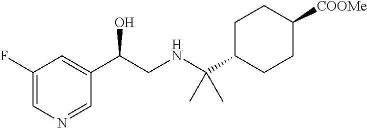 [SMILES: COC(=O)[C@H]1CC[C@H](C(C)(C)NC[C@H](O)c2cncc(F)c2)CC1] | (1R,4r)-8-[(R)-2-(5- fluoro-3-pyridyl)-2- hydroxyethylamino]-7- methoxy-p-menthan-7- one | +++ | − |
| 371 | 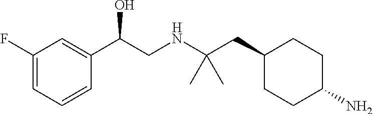 [SMILES: CC(C)(C[C@H]1CC[C@H](N)CC1)NC[C@H](O)c1cccc(F)c1] | (R)-2-{1,1-dimethyl-2- [(1r,4R)-4- aminocyclohexyl]ethyla- mino}-1-(m- fluorophenyl)-1-ethanol | +++ | − |
| 372 | 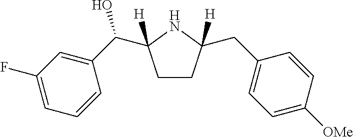 [SMILES: COc1ccc(C[C@@H]2CC[C@H]([C@@H](O)c3cccc(F)c3)N2)cc1] | (S)-{(2R,5S)-5-[(p- methoxyphenyl)methyl]- 2-pyrrolidinyl}(m- fluorophenyl)methanol | +++ | − |
| 373 | 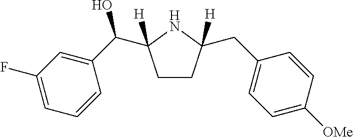 [SMILES: COc1ccc(C[C@@H]2CC[C@H]([C@H](O)c3cccc(F)c3)N2)cc1] | (R)-{(2R,5S)-5-[(p- methoxyphenyl)methyl]- 2-pyrrolidinyl}(m- fluorophenyl)methanol | +++ | + |
| 374 | 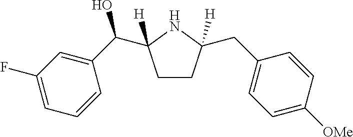 [SMILES: COc1ccc(C[C@H]2CC[C@H]([C@H](O)c3cccc(F)c3)N2)cc1] | (R)-{(2R,5R)-5-[(p- methoxyphenyl)methyl]- 2-pyrrolidinyl}(m- fluorophenyl)methanol | +++ | − |
| 375 | 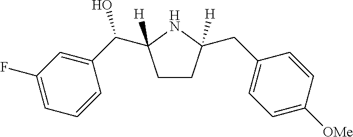 [SMILES: COc1ccc(C[C@H]2CC[C@H]([C@@H](O)c3cccc(F)c3)N2)cc1] | (S)-{(2R,5R)-5-[(p- methoxyphenyl)methyl]- 2-pyrrolidinyl}(m- fluorophenyl)methanol | +++ | − |
| 376 | 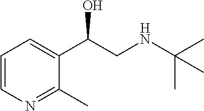 | (R)-2-(tert-butylamino)- 1-(2-methyl-3-pyridyl)- 1-ethanol | +++ | + |
| 377 | 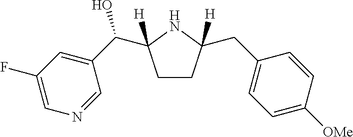 [SMILES: COc1ccc(C[C@@H]2CC[C@H]([C@@H](O)c3cncc(F)c3)N2)cc1] | (S)-{(2R,5S)-5-[(p- methoxyphenyl)methyl]- 2-pyrrolidinyl}(5- fluoro-3- pyridyl)methanol | +++ | − |
| 378 | 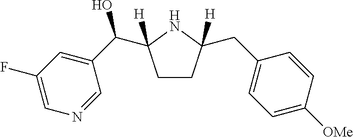 [SMILES: COc1ccc(C[C@@H]2CC[C@H]([C@H](O)c3cncc(F)c3)N2)cc1] | (R)-{(2R,5S)-5-[(p- methoxyphenyl)methyl]- 2-pyrrolidinyl}(5- fluoro-3- pyridyl)methanol | +++ | − |
| 379 | 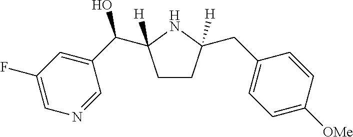 [SMILES: COc1ccc(C[C@H]2CC[C@H]([C@H](O)c3cncc(F)c3)N2)cc1] | (R)-{(2R,5R)-5-[(p- methoxyphenyl)methyl]- 2-pyrrolidinyl}(5- fluoro-3- pyridyl)methanol | +++ | − |
| 380 | 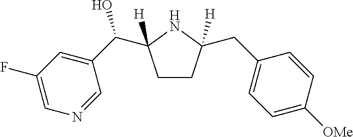 [SMILES: COc1ccc(C[C@H]2CC[C@H]([C@@H](O)c3cncc(F)c3)N2)cc1] | (S)-{(2R,5R)-5-[(p- methoxyphenyl)methyl]- 2-pyrrolidinyl}(5- fluoro-3- pyridyl)methanol | +++ | − |
| 381 | 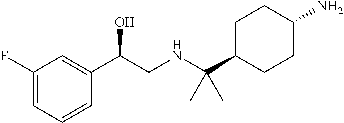 [SMILES: CC(C)(NC[C@H](O)c1cccc(F)c1)[C@H]1CC[C@H](N)CC1] | (R)-1-(m-fluorophenyl)- 2-{1-methyl-1- [(1r,4R)-4- aminocyclohexyl]ethyla- mino}-1-ethanol | ++ | − |
| 382 | 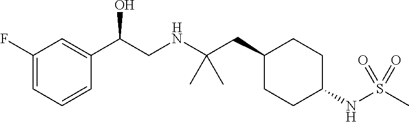 [SMILES: CC(C)(C[C@H]1CC[C@H](NS(C)(=O)=O)CC1)NC[C@H](O)c1cccc(F)c1] | (R)-2-{1,1-dimethyl-2- [(1r,4R)-4- (mesylamino)cyclohexyl] ethylamino}-1-(m- fluorophenyl)-1-ethanol | +++ | + |
| 383 | 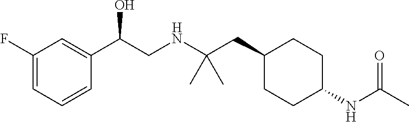 [SMILES: CC(=O)N[C@H]1CC[C@H](CC(C)(C)NC[C@H](O)c2cccc(F)c2)CC1] | N-[(1R,4r)-4-{2-[(R)-2- (m-fluorophenyl)-2- hydroxyethylamino]-2- methylpropyl}cyclohexyl] acetamide | +++ | + |
| 384 | 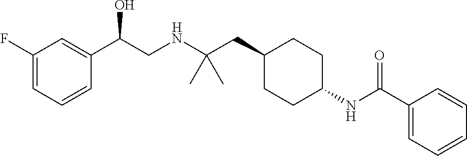 [SMILES: CC(C)(C[C@H]1CC[C@H](NC(=O)c2ccccc2)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] benzamide | +++ | +++ |
| 385 | 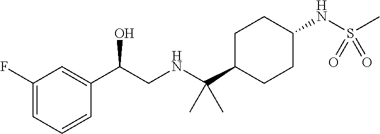 [SMILES: CC(C)(NC[C@H](O)c1cccc(F)c1)[C@H]1CC[C@H](NS(C)(=O)=O)CC1] | (R)-1-(m-fluorophenyl)- 2-{1-methyl-1- [(1r,4R)-4- (mesylamino)cyclohexyl] ethylamino}-1-ethanol | ++ | − |
| 386 | 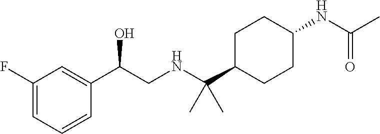 [SMILES: CC(=O)N[C@H]1CC[C@H](C(C)(C)NC[C@H](O)c2cccc(F)c2)CC1] | N-[(1R,4r)-4-{1-[(R)-2- (m-fluorophenyl)-2- hydroxyethylamino]-1- methylethyl}cyclohexyl] acetamide | +++ | − |
| 387 | 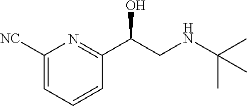 [SMILES: CC(C)(C)NC[C@H](O)c1cccc(C#N)n1] | 6-[(S)-2-(tert- butylamino)-1- hydroxyethyl]-2- pyridinecarbonitrile | +++ | + |
| 388 | 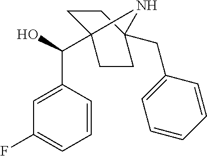 [SMILES: O[C@H](c1cccc(F)c1)C12CCC(Cc3ccccc3)(CC1)N2] | (R)-(4-benzyl-7- azabicyclo[2.2.1]hept- 1-yl)(m- fluorophenyl)methanol | +++ | + |
| 389 | 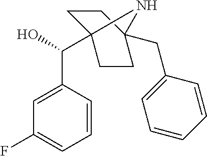 [SMILES: O[C@@H](c1cccc(F)c1)C12CCC(Cc3ccccc3)(CC1)N2] | (S)-(4-benzyl-7- azabicyclo[2.2.1]hept- 1-yl)(m- fluorophenyl)methanol | ++ | − |
| 390 | 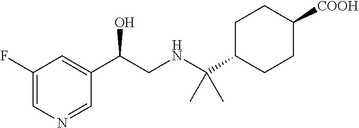 [SMILES: CC(C)(NC[C@H](O)c1cncc(F)c1)[C@H]1CC[C@H](C(=O)O)CC1] | (1R,4r)-8-[(R)-2-(5- fluoro-3-pyridyl)-2- hydroxyethylamino]-7- hydroxy-p-menthan-7- one | ++ | − |
| 391 | 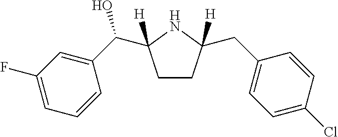 [SMILES: O[C@@H](c1cccc(F)c1)[C@H]1CC[C@@H](Cc2ccc(Cl)cc2)N1] | (S)-{(2R,5S)-5-[(p- chlorophenyl)methyl]-2- pyrrolidinyl}(m- fluorophenyl)methanol | ++ | − |
| 392 | 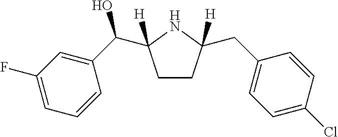 [SMILES: O[C@H](c1cccc(F)c1)[C@H]1CC[C@@H](Cc2ccc(Cl)cc2)N1] | (R)-{(2R,5S)-5-[(p- chlorophenyl)methyl]-2- pyrrolidinyl}(m- fluorophenyl)methanol | +++ | − |
| 393 | 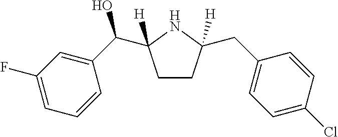 [SMILES: O[C@H](c1cccc(F)c1)[C@H]1CC[C@H](Cc2ccc(Cl)cc2)N1] | (R)-{(2R,5R)-5-[(p- chlorophenyl)methyl]-2- pyrrolidinyl}(m- fluorophenyl)methanol | +++ | − |
| 394 | 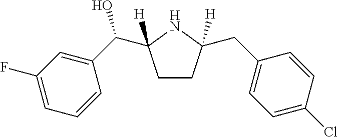 [SMILES: O[C@@H](c1cccc(F)c1)[C@H]1CC[C@H](Cc2ccc(Cl)cc2)N1] | (S)-{(2R,5R)-5-[(p- chlorophenyl)methyl]-2- pyrrolidinyl}(m- fluorophenyl)methanol | ++ | − |
| 395 | 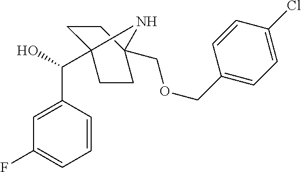 [SMILES: O[C@@H](c1cccc(F)c1)C12CCC(COCc3ccc(Cl)cc3)(CC1)N2] | (S)-(4-{[(p- chlorophenyl)methoxy] methyl}-7- azabicyclo[2.2.1]hept- 1-yl)(m- fluorophenyl)methanol | ++ | − |
| 396 | 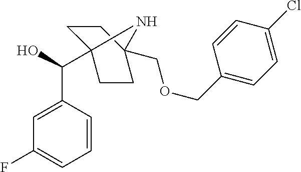 [SMILES: O[C@H](c1cccc(F)c1)C12CCC(COCc3ccc(Cl)cc3)(CC1)N2] | (R)-(4-{[(p- chlorophenyl)methoxy] methyl}-7- azabicyclo[2.2.1]hept- 1-yl)(m- fluorophenyl)methanol | +++ | − |
| 397 | 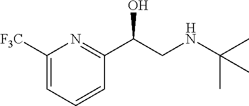 [SMILES: CC(C)(C)NC[C@H](O)c1cccc(C(F)(F)F)n1] | (S)-2-(tert-butylamino)- 1-[6-(trifluoromethyl)- 2-pyridyl]-1-ethanol | +++ | − |
| 398 | 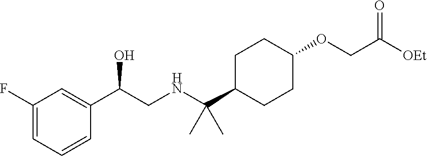 [SMILES: CCOC(=O)CO[C@H]1CC[C@H](C(C)(C)NC[C@H](O)c2cccc(F)c2)CC1] | ethyl[(1R,4r)-4-{1- [(R)-2-(m- fluorophenyl)-2- hydroxyethylamino]-1- methylethyl}cycloehxyl- oxy]acetate | +++ | − |
| 399 | 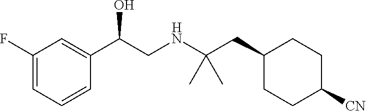 [SMILES: CC(C)(C[C@H]1CC[C@@H](C#N)CC1)NC[C@H](O)c1cccc(F)c1] | (1S,4s)-4-{2-[(R)-2- (m-fluorophenyl)-2- hydroxyethylamino]-2- methylpropyl)cyclohexane- carbonitrile | +++ | + |
| 400 | 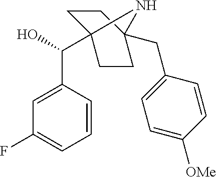 [SMILES: COc1ccc(CC23CCC([C@@H](O)c4cccc(F)c4)(CC2)N3)cc1] | (S)-(m-fluorophenyl)(4- [(p- methoxyphenyl)methyl]- 7- azabicyclo[2.2.1]hept- 1-yl}methanol | ++ | − |
| 401 | 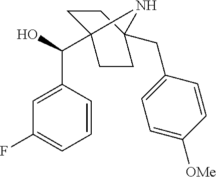 [SMILES: COc1ccc(CC23CCC([C@H](O)c4cccc(F)c4)(CC2)N3)cc1] | (R)-(m-fluorophenyl){4- [(p- methoxyphenyl)methyl]- 7- azabicyclo[2.2.1]hept- 1-yl}methanol | +++ | − |
| 402 | 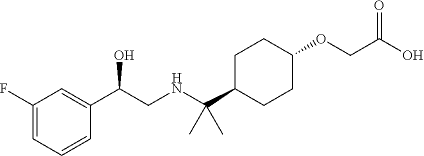 [SMILES: CC(C)(NC[C@H](O)c1cccc(F)c1)[C@H]1CC[C@H](OCC(=O)O)CC1] | [(1R,r)-4-{1-[(R)-2- (m-fluorophenyl)-2- hydroxyethylamino]-1- methylethyl}cyclohexyl- oxy]acetic acid | ++ | − |
| 403 | 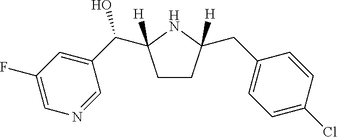 [SMILES: O[C@@H](c1cncc(F)c1)[C@H]1CC[C@@H](Cc2ccc(Cl)cc2)N1] | (S)-{(2R,5S)-5-[(p- chlorophenyl)methyl]-2- pyrroldiinyl}(5-fluoro-3- pyridyl)methanol | ++ | − |
| 404 | 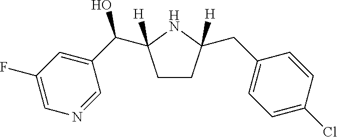 [SMILES: O[C@H](c1cncc(F)c1)[C@H]1CC[C@@H](Cc2ccc(Cl)cc2)N1] | (R)-{(2R,5S)-5-[(p- chlorophenyl)methyl]-2- pyrrolidinyl}(5-fluoro-3- pyridyl)methanol | +++ | − |
| 405 | 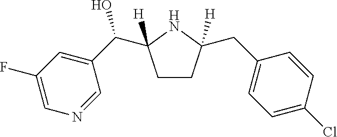 [SMILES: O[C@@H](c1cncc(F)c1)[C@H]1CC[C@H](Cc2ccc(Cl)cc2)N1] | (S)-{(2R,5R)-5-[(p- chlorophenyl)methyl]-2- pyrrolidinyl}(5-fluoro-3- pyridyl)methanol | +++ | − |
| 406 | 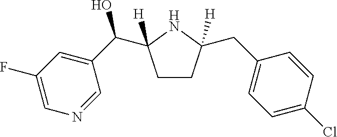 [SMILES: O[C@H](c1cncc(F)c1)[C@H]1CC[C@H](Cc2ccc(Cl)cc2)N1] | (R)-{(2R,5R)-5-[(p- chlorophenyl)methyl]-2- pyrrolidinyl}(5-fluoro-3- pyridyl)methanol | +++ | − |
| 407 | 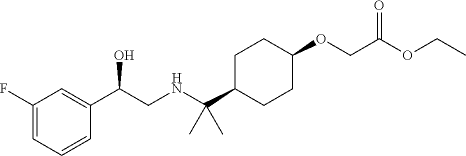 [SMILES: CCOC(=O)CO[C@H]1CC[C@@H](C(C)(C)NC[C@H](O)c2cccc(F)c2)CC1] | ethyl [(1S,4s)-4-{1- [(R)-2-(m- fluorophenyl)-2- hydroxyethylamino]-1- methylethyl}cyclohexyl- oxy]acetate | ++ | − |
| 408 | 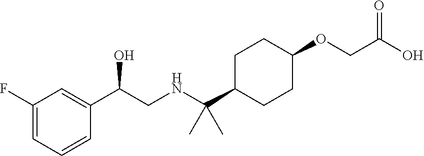 [SMILES: CC(C)(NC[C@H](O)c1cccc(F)c1)[C@H]1CC[C@@H](OCC(=O)O)CC1] | [(1S,4s)-4-{1-[(R)-2- (m-fluorophenyl)-2- hydroxyethylamino]-1- methylethyl}cyclohexyl- oxy]acetic acid | ++ | − |
| 409 | 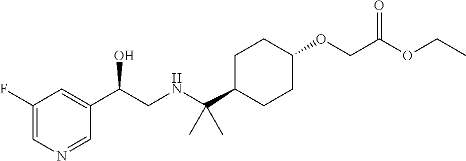 [SMILES: CCOC(=O)CO[C@H]1CC[C@H](C(C)(C)NC[C@H](O)c2cncc(F)c2)CC1] | ethyl [(1R,4r)-4-{1- [(R)-2-(5-fluoro-3- pyridyl)-2- hydroxyethylamino]-1- methylethyl}cyclohexyl- oxy]acetate | +++ | − |
| 410 | 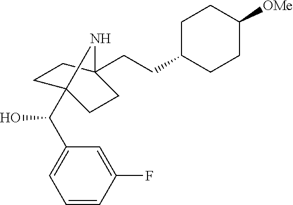 [SMILES: CO[C@H]1CC[C@H](CCC23CCC([C@@H](O)c4cccc(F)c4)(CC2)N3)CC1] | (S)-(m-fluorophenyl)(4- {2-[(1r,4R)-4- methoxycyclohexyl]ethyl}- 7- azabicyclo[2.2.1]hept- 1-yl)methanol | ++ | − |
| 411 | 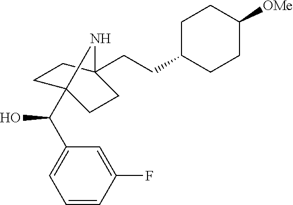 [SMILES: CO[C@H]1CC[C@H](CCC23CCC([C@H](O)c4cccc(F)c4)(CC2)N3)CC1] | (R)-(m-fluorophenyl)(4- {2-[(1r,4S)-4- methoxycyclohexyl]ethyl}- 7- azabicyclo[2.2.1]hept- 1-yl)methanol | +++ | ++ |
| 412 | 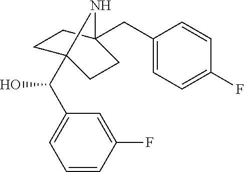 [SMILES: O[C@@H](c1cccc(F)c1)C12CCC(Cc3ccc(F)cc3)(CC1)N2] | (S)-(m-fluorophenyl){4- [(p- fluorophenyl)methyl]-7- azabicyclo[2.2.1]hept- 1-yl}methanol | ++ | − |
| 413 | 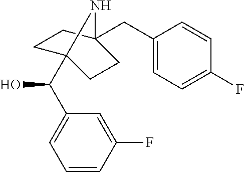 [SMILES: O[C@H](c1cccc(F)c1)C12CCC(Cc3ccc(F)cc3)(CC1)N2] | (R)-(m-florophenyl){4- [(p- fluorophenyl)methyl]-7- azabicyclo[2.2.1]hept- 1-yl}methanol | +++ | − |
| 414 | 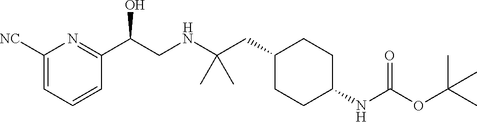 [SMILES: CC(C)(C[C@H]1CC[C@@H](NC(=O)OC(C)(C)C)CC1)NC[C@H](O)c1cccc(C#N)n1] | (1R,4s)-4-{2-[(S)-2-(6- cyano-2-pyridyl)-2- hydroxyethylamino]-2- methylpropyl}cyclohexyl 2-methyl-2- propanecarbamate | ++ | − |
| 415 | 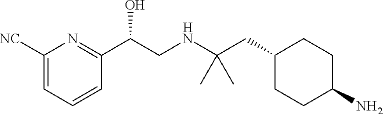 [SMILES: CC(C)(C[C@H]1CC[C@H](N)CC1)NC[C@@H](O)c1cccc(C#N)n1] | 6-[(R)-2-{1,1-dimethyl- 2-[(1r,4R)-4- aminocyclohexyl]ethyla- mino}-1-hydroxyethyl]- 2-pyridinecarbonitrile | ++ | − |
| 416 | 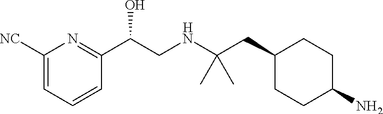 [SMILES: CC(C)(C[C@H]1CC[C@@H](N)CC1)NC[C@@H](O)c1cccc(C#N)n1] | 6-[(R)-2-{1,1-dimethyl- 2-[(1s,4S)-4- aminocyclohexyl]ethyla- mino}-1-hydroxyethyl]- 2-pyridinecarbonitrile | ++ | − |
| 417 | 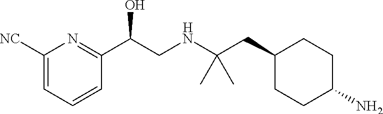 [SMILES: CC(C)(C[C@H]1CC[C@H](N)CC1)NC[C@H](O)c1cccc(C#N)n1] | 6-[(S)-2-{1,1-dimethyl- 2-[(1r,4S)-4- aminocyclohexyl]ethyla- mino}-1-hydroxyethyl]- 2-pyridinecarbontirile | +++ | + |
| 418 | 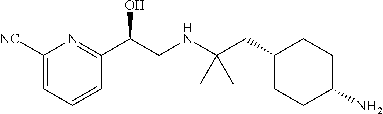 [SMILES: CC(C)(C[C@H]1CC[C@@H](N)CC1)NC[C@H](O)c1cccc(C#N)n1] | 6-[(S)-2-{1,1-dimethyl- 2-[(1s,4R)-4- aminocyclohexyl]ethyla- mino}-1-hydroxyethyl]- 2-pyridinecarbonitrile | ++ | − |
| 419 | 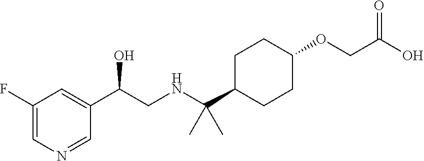 [SMILES: CC(C)(NC[C@H](O)c1cncc(F)c1)[C@H]1CC[C@H](OCC(=O)O)CC1] | [(1R,4r)-4-{1-[(R)-2- (5-fluoro-3-pyridyl)-2- hydroxyethylamino]-1- methylethyl}cyclohexyl- oxy]aetic acid | ++ | − |
| 420 | 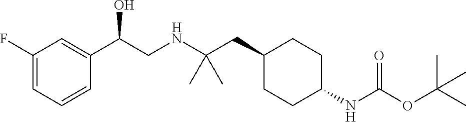 [SMILES: CC(C)(C[C@H]1CC[C@H](NC(=O)OC(C)(C)C)CC1)NC[C@H](O)c1cccc(F)c1] | (1R,4r)-4-{2-[(R)-2- (m-fluorophenyl)-2- hydroxyethylamino]-2- methylpropyl}cyclohexyl 2-methyl-2- propanecarbamate | ++ | + |
| 421 | 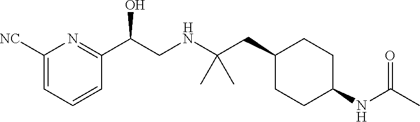 [SMILES: CC(=O)N[C@H]1CC[C@@H](CC(C)(C)NC[C@H](O)c2cccc(C#N)n2)CC1] | N-[(1R,4s)-4-{2-[(S)-2- (6-cyano-2-pyridyl)-2- hydroxyethylamino]-2- methylpropyl}cyclohexyl] acetamide | +++ | − |
| 422 | 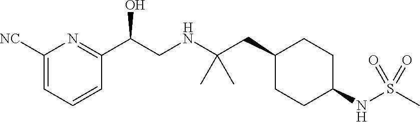 [SMILES: CC(C)(C[C@H]1CC[C@@H](NS(C)(=O)=O)CC1)NC[C@H](O)c1cccc(C#N)n1] | 6-[(S)-2-{1,1-dimethyl- 2-[(1s,4R)-4- (mesylamino)cylcohexyl] ethylamino}-1- hydroxyethyl]-2- pyridinecarbonitrile | ++ | − |
| 423 | 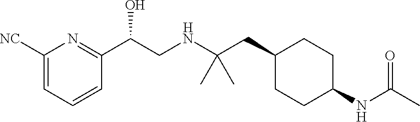 [SMILES: CC(=O)N[C@H]1CC[C@@H](CC(C)(C)NC[C@@H](O)c2cccc(C#N)n2)CC1] | N-[(1S,4s)-4-{2-[(R)-2- (6-cyano-2-pyridyl)-2- hydroxyethylamino]-2- methylpropyl}cyclohexyl] acetamide | − | − |
| 424 | 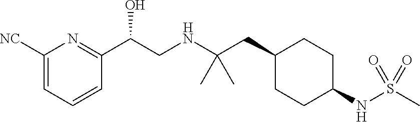 [SMILES: CC(C)(C[C@H]1CC[C@@H](NS(C)(=O)=O)CC1)NC[C@@H](O)c1cccc(C#N)n1] | 6-[(R)-2-{1,1-dimethyl- 2-[(1s,4S)-4- (mesylamino)cyclohexyl] ethylamino}-1- hydroxyethyl]-2- pyridinecarbonitrile | + | − |
| 425 | 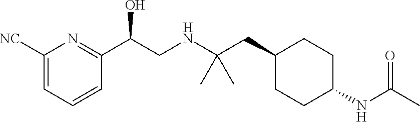 [SMILES: CC(=O)N[C@H]1CC[C@H](CC(C)(C)NC[C@H](O)c2cccc(C#N)n2)CC1] | N-[(1S,4r)-4-{2-[(S)-2- (6-cyano-2-pyridyl)-2- hydroxyethylamino]-2- methylpropyl}cyclohexyl] acetamide | +++ | + |
| 426 | 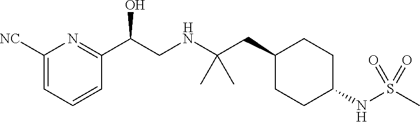 [SMILES: CC(C)(C[C@H]1CC[C@H](NS(C)(=O)=O)CC1)NC[C@H](O)c1cccc(C#N)n1] | 6-[(S)-2-{1,1-dimethyl- 2-[(1r,4S)-4- (mesylamino)cyclohexyl] ethylamino}-1- hydroxyethyl]-2- pyridinecarbonitrile | +++ | − |
| 427 | 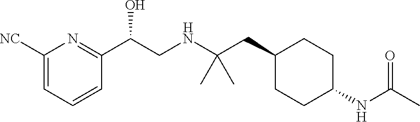 [SMILES: CC(=O)N[C@H]1CC[C@H](CC(C)(C)NC[C@@H](O)c2cccc(C#N)n2)CC1] | N-[(1R,4r)-4-{2-[(R)-2- (6-cyano-2-pyridyl)-2- hydroxyethylamino]-2- methylpropyl}cyclohexyl] acetamide | − | − |
| 428 | 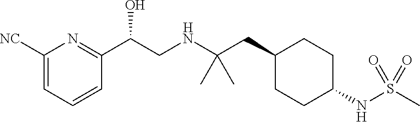 [SMILES: CC(C)(C[C@H]1CC[C@H](NS(C)(=O)=O)CC1)NC[C@@H](O)c1cccc(C#N)n1] | 6-[(R)-2-{1,1-dimethyl- 2-[(1r,4R)-4- (mesylamino)cyclohexyl] ethylamino}-1- hydroxyethyl]-2- pyridinecarobnitrile | − | − |
| 429 | 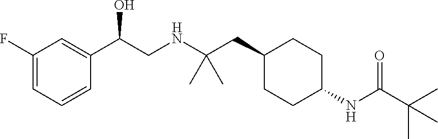 [SMILES: CC(C)(C[C@H]1CC[C@H](NC(=O)C(C)(C)C)CC1)NC[C@H](O)c1cccc(F)c1] | N-[(1R,4r)-4-{2-[(R)-2- (m-fluorophenyl)-2- hdyroxyethylamino]-2- methylpropyl}cyclohexyl] 2,2- dimethylpropionamide | +++ | + |
| 430 | 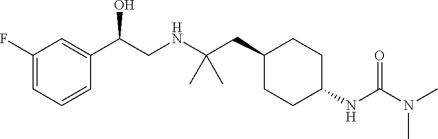 [SMILES: CN(C)C(=O)N[C@H]1CC[C@H](CC(C)(C)NC[C@H](O)c2cccc(F)c2)CC1] | (R)-2-{1,1-dimethyl-2- [(1r,4R)-4-(3,3- dimethylureido)cyclohex- yl]ethylamino}-1-(m- fluorophenyl)-1-ethanol | +++ | ++ |
| 431 | 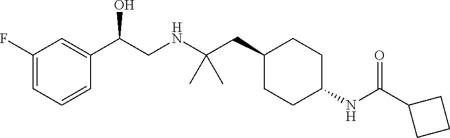 [SMILES: CC(C)(C[C@H]1CC[C@H](NC(=O)C2CCC2)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] cyclobutanecarboxamide | +++ | ++ |
| 432 | 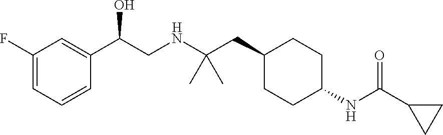 [SMILES: CC(C)(C[C@H]1CC[C@H](NC(=O)C2CC2)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] cyclopropanecarboxmaide | +++ | +++ |
| 433 | 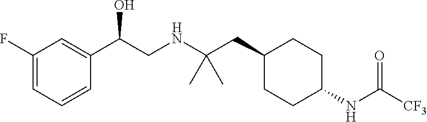 [SMILES: CC(C)(C[C@H]1CC[C@H](NC(=O)C(F)(F)F)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] trifluoroacetamide | +++ | + |
| 434 | 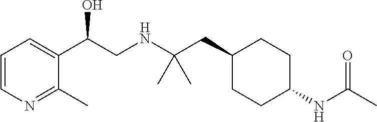 [SMILES: CC(=O)N[C@H]1CC[C@H](CC(C)(C)NC[C@H](O)c2cccnc2C)CC1] | N-[(1R,4r)-4-{2-[(R)-2- hydroxy-2-(2-methyl-3- pyridyl)ethylamino]-2- methylpropyl}cyclohexyl] acetamide | +++ | − |
| 435 | 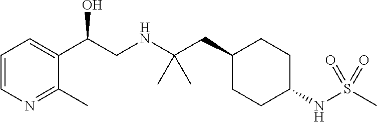 [SMILES: Cc1ncccc1[C@@H](O)CNC(C)(C)C[C@H]1CC[C@H](NS(C)(=O)=O)CC1] | (R)-2-{1,1-dimethyl-2- [(1r,4R)-4- (mesylamino)cyclohexyl] ethylamino}-1-(2- methyl-3-pyridyl)-1- ethanol | + | − |
| 436 | 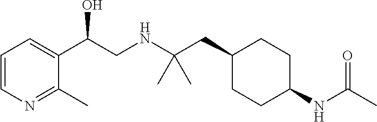 [SMILES: CC(=O)N[C@H]1CC[C@@H](CC(C)(C)NC[C@H](O)c2cccnc2C)CC1] | N-[(1S,4s)-4-{2-[(R)-2- hydroxy-2-(2-methyl-3- pyridyl)ethylamino]-2- methylpropyl}cyclohexyl] acetamide | ++ | − |
| 437 | 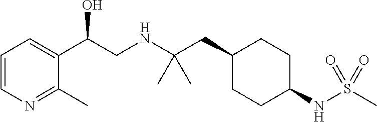 [SMILES: Cc1ncccc1[C@@H](O)CNC(C)(C)C[C@H]1CC[C@@H](NS(C)(=O)=O)CC1] | (R)-2-{1,1-dimethyl-2- [(1s,4S)-4- (mesylamino)cyclohexyl] ethylamino}-1-(2- methyl-3-pyridyl)-1- ethanol | + | − |
| 438 | 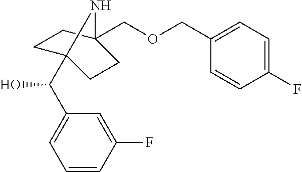 [SMILES: O[C@@H](c1cccc(F)c1)C12CCC(COCc3ccc(F)cc3)(CC1)N2] | (S)-(m-fluorophenyl)(4- {[(p- fluorophenyl)methoxy] methyl}-7- azabicyclo[2.2.1]hept- 1-yl)methanol | − | − |
| 439 | 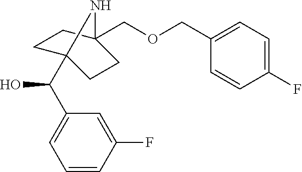 [SMILES: O[C@H](c1cccc(F)c1)C12CCC(COCc3ccc(F)cc3)(CC1)N2] | (R)-(m-fluorophenyl)(4- {[(p- fluorophenyl)methoxy] methyl}-7- azabicyclo[2.2.1]hept- 1-yl)methanol | +++ | + |
| 440 | 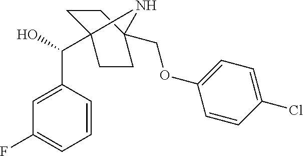 [SMILES: O[C@@H](c1cccc(F)c1)C12CCC(COc3ccc(Cl)cc3)(CC1)N2] | (S)-{4-[(p- chlorophenoxy)methyl]- 7- azabicyclo[2.2.1]hept- 1-yl}(m- fluorophenyl)methanol | +++ | − |
| 441 | 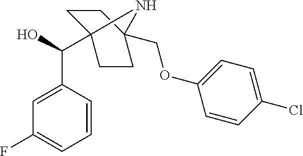 [SMILES: O[C@H](c1cccc(F)c1)C12CCC(COc3ccc(Cl)cc3)(CC1)N2] | (R)-{4-[(p- chlorophenoxy)methyl]- 7- azabicyclo[2.2.1]hept- 1-yl}(m- fluorophenyl)methanol | +++ | − |
| 442 | 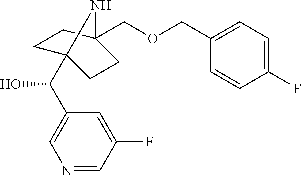 [SMILES: O[C@@H](c1cncc(F)c1)C12CCC(COCc3ccc(F)cc3)(CC1)N2] | (S)-(4-{[(p- fluorophenyl)methoxy] methyl}-7- azabicyclo[2.2.1]hept- 1-yl)(5-fluoro-3- pyridyl)methanol | + | − |
| 443 | 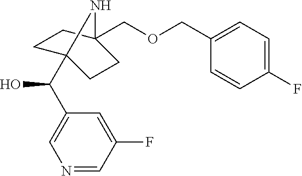 [SMILES: O[C@H](c1cncc(F)c1)C12CCC(COCc3ccc(F)cc3)(CC1)N2] | (R)-(4-{[(p- fluorophenyl)methoxy] methyl}-7- azabicyclo[2.2.1]hept- 1-yl)(5-fluoro-3- pyridyl)methanol | +++ | + |
| 444 | 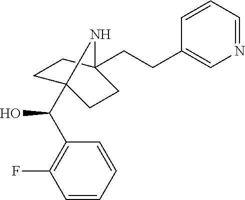 [SMILES: O[C@H](c1ccccc1F)C12CCC(CCc3cccnc3)(CC1)N2] | (R)-(o-fluorophenyl){4- [2-(3-pyridyl)ethyl]-7- azabicyclo[2.2.1]hept- 1-yl}methanol | +++ | ++ |
| 445 | 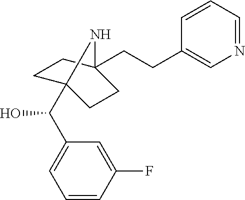 [SMILES: O[C@@H](c1cccc(F)c1)C12CCC(CCc3cccnc3)(CC1)N2] | (S)-(m-fluorophenyl){4- [2-(3-pyridyl)ethyl]-7- azabicyclo[2.2.1]hept- 1-yl}methanol | +++ | − |
| 446 | 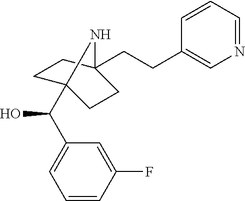 [SMILES: O[C@H](c1cccc(F)c1)C12CCC(CCc3cccnc3)(CC1)N2] | (R)-(m-fluorophenyl){4- [2-(3-pyridyl)ethyl]-7- azabicyclo[2.2.1]hept- 1-yl}methanol | +++ | ++ |
| 447 | 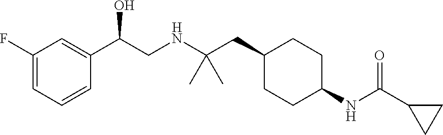 [SMILES: CC(C)(C[C@H]1CC[C@@H](NC(=O)C2CC2)CC1)NC[C@H](O)c1cccc(F)c1] | N-[(1S,4s)-4-{2-[(R)-2- (m-fluorophenyl)-2- hydroxyethylamino]-2- methylpropyl}cyclohexyl] cyclopropanecarboxamide | +++ | − |
| 448 | 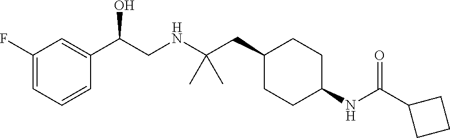 [SMILES: CC(C)(C[C@H]1CC[C@@H](NC(=O)C2CCC2)CC1)NC[C@H](O)c1cccc(F)c1] | N-[(1S,4s)-4-{2-[(R)-2- (m-fluorophenyl)-2- hydroxyethylamino]-2- methylpropyl}cyclohexyl] cyclobutanecarboxamide | ++ | − |
| 449 | 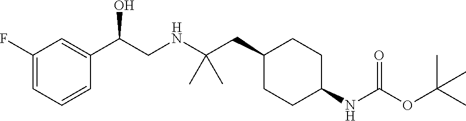 [SMILES: CC(C)(C[C@H]1CC[C@@H](NC(=O)OC(C)(C)C)CC1)NC[C@H](O)c1cccc(F)c1] | (1S,4s)-4-{2-[(R)-2- (m-fluorophenyl)-2- hydroxyethylamino]-2- methylpropyl}cyclohexyl 2-methyl-2- propanecarbamate | ++ | − |
| 450 | 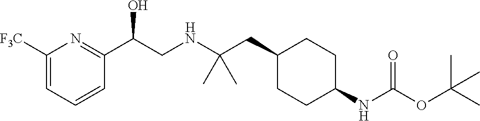 [SMILES: CC(C)(C[C@H]1CC[C@@H](NC(=O)OC(C)(C)C)CC1)NC[C@H](O)c1cccc(C(F)(F)F)n1] | (1R,4s)-4-(2-{(S)-2- hydroxy-2-[6- (trifluoromethyl)-2- pyridyl]ethylamino}-2- methylpropyl)cyclohexyl 2-methyl-2- propanecarbamate | − | − |
| 451 | 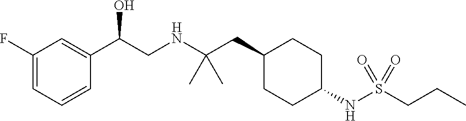 [SMILES: CCCS(=O)(=O)N[C@H]1CC[C@H](CC(C)(C)NC[C@H](O)c2cccc(F)c2)CC1] | (R)-2-{1,1-dimethyl-2- [(1r,4R)-4- (propylsulfonylamino)cy- clohexyl]ethylamino}-1- (m-fluorophenyl)-1- ethanol | ++ | + |
| 452 | 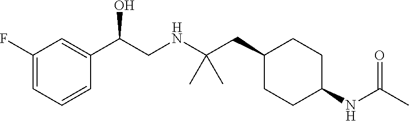 [SMILES: CC(=O)N[C@H]1CC[C@@H](CC(C)(C)NC[C@H](O)c2cccc(F)c2)CC1] | N-[(1S,4s)-4-{2-[(R)-2- (m-fluorophenyl)-2- hydroxyethylamino]-2- methylpropyl}cyclohexyl] acetamide | +++ | − |
| 453 | 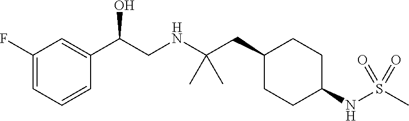 [SMILES: CC(C)(C[C@H]1CC[C@@H](NS(C)(=O)=O)CC1)NC[C@H](O)c1cccc(F)c1] | (R)-2-{1,1-dimethyl-2- [(1s,4S)-4- (mesylamino)cyclohexyl] ethylamino}-1-(m- fluorophenyl)-1-ethanol | +++ | − |
| 454 | 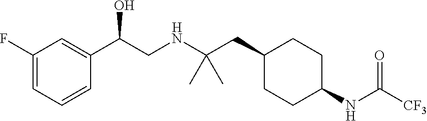 [SMILES: CC(C)(C[C@H]1CC[C@@H](NC(=O)C(F)(F)F)CC1)NC[C@H](O)c1cccc(F)c1] | N-[(1S,4s)-4-{2-[(R)-2- (m-fluorophenyl)-2- hydroxyethylamino]-2- methylpropyl}cyclohexyl] trifluoroacetamide | +++ | − |
| 455 | 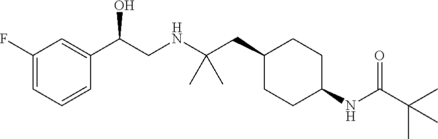 [SMILES: CC(C)(C[C@H]1CC[C@@H](NC(=O)C(C)(C)C)CC1)NC[C@H](O)c1cccc(F)c1] | N-[(1S,4s)-4-{2-[(R)-2- (m-fluorophenyl)-2- hydroxyethylamino]-2- methylpropyl}cyclohexyl] 2,2- dimethylpropionamide | ++ | − |
| 456 | 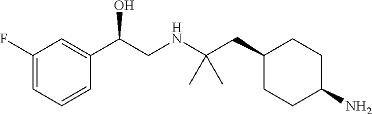 [SMILES: CC(C)(C[C@H]1CC[C@@H](N)CC1)NC[C@H](O)c1cccc(F)c1] | (R)-2-{1,1-dimethyl-2- [(1s,4S)-4- aminocyclohexyl]ethyla- mino}-1-(m- fluorophenyl)-1-ethanol | +++ | − |
| 457 | 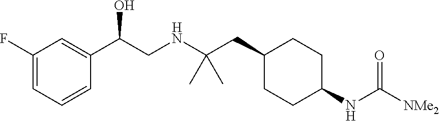 [SMILES: CN(C)C(=O)N[C@H]1CC[C@@H](CC(C)(C)NC[C@H](O)c2cccc(F)c2)CC1] | (R)-2-{1,1-dimethyl-2- [(1s,4S)-4-(3,3- dimethylureido)cyclohex- yl]ethylamino}-1-(m- fluorophenyl)-1-ethanol | +++ | + |
| 458 | 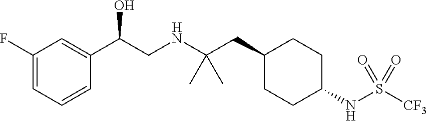 [SMILES: CC(C)(C[C@H]1CC[C@H](NS(=O)(=O)C(F)(F)F)CC1)NC[C@H](O)c1cccc(F)c1] | (R)-2-{1,1-dimethyl-2- [(1r,4R)-4- (trifluoromesylamino)cy- clohexyl]ethylamino}-1- (m-fluorophenyl)-1- ethanol | +++ | − |
| 459 | 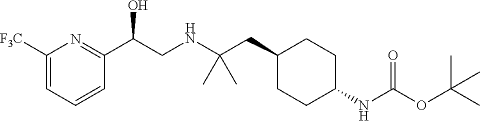 [SMILES: CC(C)(C[C@H]1CC[C@H](NC(=O)OC(C)(C)C)CC1)NC[C@H](O)c1cccc(C(F)(F)F)n1] | (1S,4r)-4-(2-{(S)-2- hydroxy-2-[6- (trifluoromethyl)-2- pyridyl]ethylamino}-2- methylpropyl)cyclohexyl 2-methyl-2- propanecabamate | +++ | − |
| 460 | 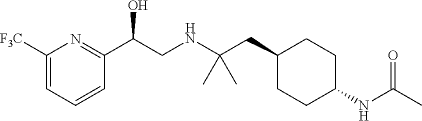 [SMILES: CC(=O)N[C@H]1CC[C@H](CC(C)(C)NC[C@H](O)c2cccc(C(F)(F)F)n2)CC1] | N-[(1S,4r)-4-(2-{(S)-2- hydroxy-2-[6- (trifluoromethyl)-2- pyridyl]ethylamino}-2- methylpropyl)cyclohexyl] acetamide | ++ | − |
| 461 | 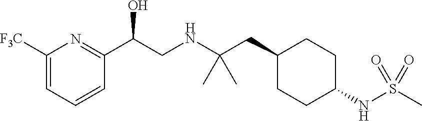 [SMILES: CC(C)(C[C@H]1CC[C@H](NS(C)(=O)=O)CC1)NC[C@H](O)c1cccc(C(F)(F)F)n1] | (S)-2-{1,1-dimethyl-2- [(1r,4S)-4- (mesylamino)cyclohexyl] ethylamino}-1-[6- (trifluoromethyl)-2- pyridyl]-1-ethanol | + | − |
| 462 | 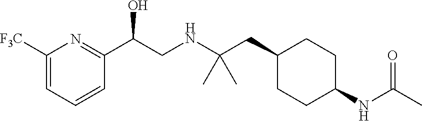 [SMILES: CC(=O)N[C@H]1CC[C@@H](CC(C)(C)NC[C@H](O)c2cccc(C(F)(F)F)n2)CC1] | N-[(1R,4s)-4-(2-{(S)-2- hydroxy-2-[6- (trifluoromethyl)-2- pyridyl]ethylamino}-2- methylpropyl)cyclohexyl] acetamide | + | − |
| 463 | 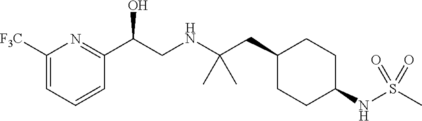 [SMILES: CC(C)(C[C@H]1CC[C@@H](NS(C)(=O)=O)CC1)NC[C@H](O)c1cccc(C(F)(F)F)n1] | (S)-2-{1,1-dimethyl-2- [(1s,4R)-4- (mesylamino)cyclohexyl] ethylamino}-1-[6- (trifluoromethyl)-2- pyridyl]-1-ethanol | ++ | − |
| 464 | 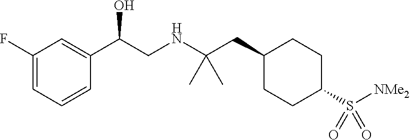 [SMILES: CN(C)S(=O)(=O)[C@H]1CC[C@H](CC(C)(C)NC[C@H](O)c2cccc(F)c2)CC1] | (R)-2-{1,1-dimethyl-2- [(1r,4R)-4- (dimethylaminosulfonyl) cyclohexyl]ethylamino}- 1-(m-fluorophenyl)-1- ethanol | + | + |
| 465 | 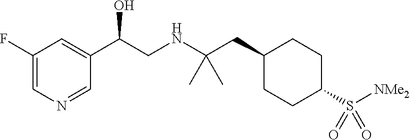 [SMILES: CN(C)S(=O)(=O)[C@H]1CC[C@H](CC(C)(C)NC[C@H](O)c2cncc(F)c2)CC1] | (R)-2-{1,1-dimethyl-2- [(1r,4R)-4- (dimethylaminosulfonyl) cyclohexyl]ethylamino}- 1-(5-fluoro-3-pyridyl)- 1-ethanol | ++ | + |
| 466 | 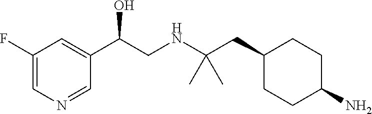 [SMILES: CC(C)(C[C@H]1CC[C@@H](N)CC1)NC[C@H](O)c1cncc(F)c1] | (R)-2-{1,1-dimethyl-2- [(1s,4S)-4- aminocyclohexyl]ethyla- mino}-1-(5-fluoro-3- pyridyl)-1-ethanol | ++ | − |
| 467 | 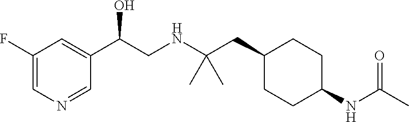 [SMILES: CC(=O)N[C@H]1CC[C@@H](CC(C)(C)NC[C@H](O)c2cncc(F)c2)CC1] | N-[(1S,4s)-4-{2-[(R)-2- (5-fluoro-3-pyridyl)-2- hydroxyethylamino]-2- methylpropyl}cyclohexyl] acetamide | +++ | − |
| 468 | 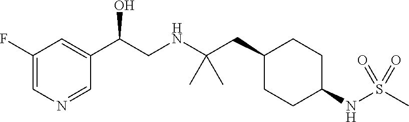 [SMILES: CC(C)(C[C@H]1CC[C@@H](NS(C)(=O)=O)CC1)NC[C@H](O)c1cncc(F)c1] | (R)-2-{1,1-dimethyl-2- [(1s,4S)-4- (mesylamino)cyclohexyl] ethylamino}-1-(5- fluoro-3-pyridyl)-1- ethanol | +++ | − |
| 469 | 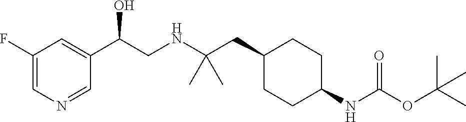 [SMILES: CC(C)(C[C@H]1CC[C@@H](NC(=O)OC(C)(C)C)CC1)NC[C@H](O)c1cncc(F)c1] | (1S,4s)-4-{2-[(R)-2-(5- fluoro-3-pyridyl)-2- hydroxyethylamino]-2- methylpropyl}cyclohexyl 2-methyl-2- propanecarbamate | +++ | − |
| 470 | 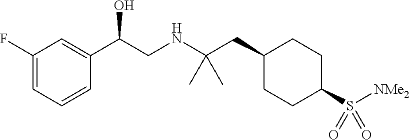 [SMILES: CN(C)S(=O)(=O)[C@H]1CC[C@@H](CC(C)(C)NC[C@H](O)c2cccc(F)c2)CC1] | (R)-2-{1,1-dimethyl-2- [(1s,4S)-4- (dimethylaminosulfonyl) cyclohexyl]ethylamino}- 1-(m-fluorophenyl)-1- ethanol | +++ | + |
| 471 | 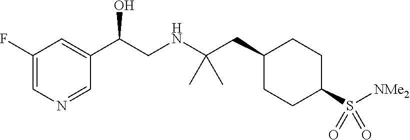 [SMILES: CN(C)S(=O)(=O)[C@H]1CC[C@@H](CC(C)(C)NC[C@H](O)c2cncc(F)c2)CC1] | (R)-2-{1,1-dimethyl-2- [(1s,4S)-4- (dimethylaminosulfonyl) cyclohexyl]ethylamino}- 1-(5-fluoro-3-pyridyl)- 1-ethanol | +++ | ++ |
| 472 | 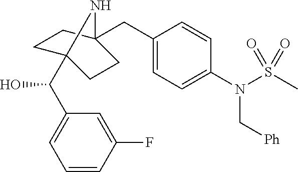 [SMILES: CS(=O)(=O)N(Cc1ccccc1)c1ccc(CC23CCC([C@@H](O)c4cccc(F)c4)(CC2)N3)cc1] | (S)-[4-({p-[(benzyl)-N- mesylamino]phenyl}meth- yl)-7- azabicyclo[2.2.1]hept- 1-yl](m- fluorophenyl)methanol | + | − |
| 473 | 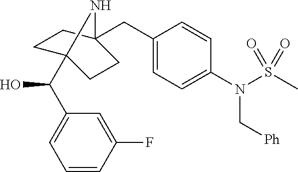 [SMILES: CS(=O)(=O)N(Cc1ccccc1)c1ccc(CC23CCC([C@H](O)c4cccc(F)c4)(CC2)N3)cc1] | (R)-[4-({p-[(benzyl)-N- mesylamino]phenyl}meth- yl)-7- azabicyclo[2.2.1]hept- 1-yl](m- fluorophenyl)methanol | + | − |
| 474 | 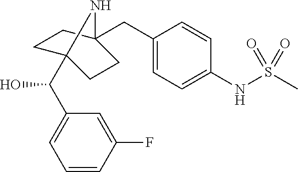 [SMILES: CS(=O)(=O)Nc1ccc(CC23CCC([C@@H](O)c4cccc(F)c4)(CC2)N3)cc1] | (S)-(m-fluorophenyl)(4- {[p- (mesylamino)phenyl]meth- yl}-7- azabicyclo[2.2.1]hept- 1-yl)methanol | ++ | − |
| 475 | 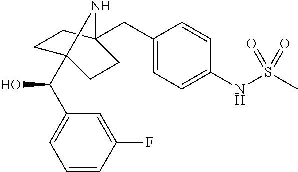 [SMILES: CS(=O)(=O)Nc1ccc(CC23CCC([C@H](O)c4cccc(F)c4)(CC2)N3)cc1] | (R)-(m-fluorophenyl)(4- {[p- (mesylamino)phenyl]meth- yl}-7- azabicyclo[2.2.1]hept- 1-yl)methanol | ++ | − |
| 476 | 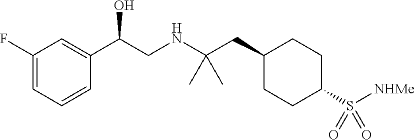 [SMILES: CNS(=O)(=O)[C@H]1CC[C@H](CC(C)(C)NC[C@H](O)c2cccc(F)c2)CC1] | (R)-2-{1,1-dimethyl-2- [(1r,4R)-4- (methylaminosulfonyl)cy- clohexyl]ethylamino}- 1-(m-fluorophenyl)-1- ethanol | +++ | + |
| 477 | 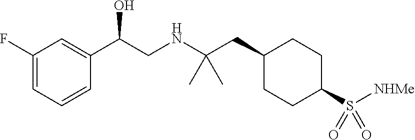 [SMILES: CNS(=O)(=O)[C@H]1CC[C@@H](CC(C)(C)NC[C@H](O)c2cccc(F)c2)CC1] | (R)-2-{1,1-dimethyl-2- [(1s,4S)-4- (methylaminosulfonyl)cy- clohexyl]ethylamino}- 1-(m-fluorophenyl)-1- ethanol | ++ | − |
| 478 | 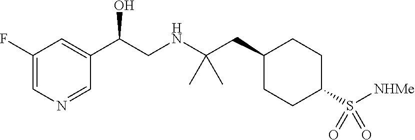 [SMILES: CNS(=O)(=O)[C@H]1CC[C@H](CC(C)(C)NC[C@H](O)c2cncc(F)c2)CC1] | (R)-2-{1,1-dimethyl-2- [(1r,4R)-4- (methylaminosulfonyl)cy- clohexyl]ethylamino}- 1-(5-fluoro-3-pyridyl)- 1-ethanol | + | − |
| 479 | 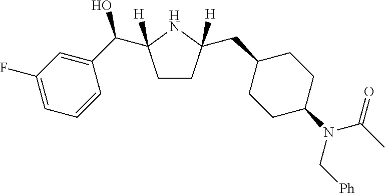 [SMILES: CC(=O)N(Cc1ccccc1)[C@H]1CC[C@@H](C[C@@H]2CC[C@H]([C@H](O)c3cccc(F)c3)N2)CC1] | N-[(1R,4s)-4- ({(2S,5R)-5-[(S)-(m- fluorophenyl)hydroxymeth- yl]-2- pyrrolidinyl}methyl)cyclo- hexyl]-N- benzylacetamide | − | − |
| 480 | 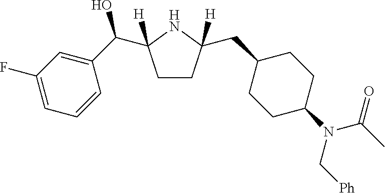 [SMILES: CC(=O)N(Cc1ccccc1)[C@H]1CC[C@@H](C[C@@H]2CC[C@H]([C@H](O)c3cccc(F)c3)N2)CC1] | N-[(1R,4s)-4- ({(2S,5R)-5-[(R)-(m- fluorophenyl)hydroxymeth- yl]-2- pyrrolidinyl}methyl)cyclo- hexyl]-N- benzylacetamide | − | − |
| 481 | 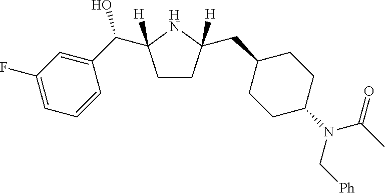 [SMILES: CC(=O)N(Cc1ccccc1)[C@H]1CC[C@H](C[C@@H]2CC[C@H]([C@@H](O)c3cccc(F)c3)N2)CC1] | N-[(1S,4r)-4-({(2S,5R)- 5-[(S)-(m- fluorophenyl)hydroxymeth- yl]-2- pyrrolidinyl}methyl)cyclo- hexyl]-N- benzylacetamide | − | − |
| 482 | 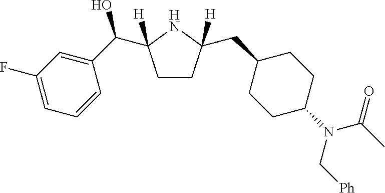 [SMILES: CC(=O)N(Cc1ccccc1)[C@H]1CC[C@H](C[C@@H]2CC[C@H]([C@H](O)c3cccc(F)c3)N2)CC1] | N-[(1S,4r)-4-({(2S,5R)- 5-[(R)-(m- fluorophenyl)hydroxymeth- yl]-2- pyrrolidinyl}methyl)cyclo- hexyl]-N- benzylacetamide | − | − |
| 483 | 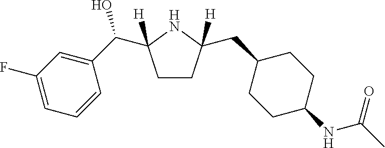 [SMILES: CC(=O)N[C@H]1CC[C@@H](C[C@@H]2CC[C@H]([C@@H](O)c3cccc(F)c3)N2)CC1] | N-[(1R,4s)-4- ({(2S,5R)-5-[(S)-(m- fluorophenyl)hydroxymeth- yl]-2- pyrrolidinyl}methyl)cyclo- hexyl]acetamide | − | − |
| 484 | 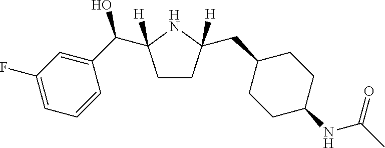 [SMILES: CC(=O)N[C@H]1CC[C@@H](C[C@@H]2CC[C@H]([C@H](O)c3cccc(F)c3)N2)CC1] | N-[(1R,4s)-4- ({(2S,5R)-5-[(R)-(m- fluorophenyl)hydroxymeth- yl]-2- pyrrolidinyl}methyl)cyclo- hexyl]acetamide | ++ | − |
| 485 | 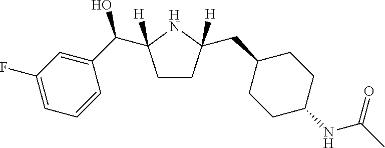 [SMILES: CC(=O)N[C@H]1CC[C@H](C[C@@H]2CC[C@H]([C@H](O)c3cccc(F)c3)N2)CC1] | N-[(1S,4r)-4-({(2S,5R)- 5-[(R)-(m- fluorophenyl)hydroxymeth- yl]-2- pyrrolidinyl}methyl)cyclo- hexyl]acetamide | ++ | − |
| 486 | 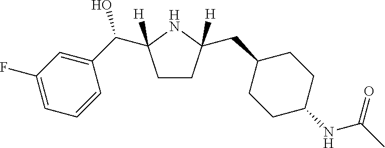 [SMILES: CC(=O)N[C@H]1CC[C@H](C[C@@H]2CC[C@H]([C@@H](O)c3cccc(F)c3)N2)CC1] | N-[(1S,4r)-4-({(2S,5R)- 5-[(S)-(m- fluorophenyl)hydroxymeth- yl]-2- pyrrolidinyl}methyl)cyclo- hexyl]acetamide | ++ | − |
| 487 | 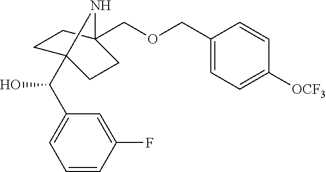 [SMILES: O[C@@H](c1cccc(F)c1)C12CCC(COCc3ccc(OC(F)(F)F)cc3)(CC1)N2] | (S)-(m-fluorophenyl)(4- {[(p- trifluoromethoxyphenyl) methoxy]methyl}-7- azabicylco[2.2.1]hept- 1-yl)methanol | − | − |
| 488 | 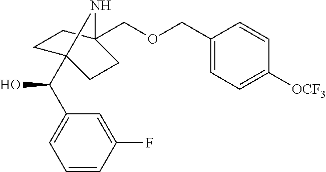 [SMILES: O[C@H](c1cccc(F)c1)C12CCC(COCc3ccc(OC(F)(F)F)cc3)(CC1)N2] | (R)-(m-fluorophenyl)(4- {[(p- trifluoromethoxyphenyl) methoxy]methyl}-7- azabicyclo[2.2.1]hept- 1-yl)methanol | − | − |
| 489 | 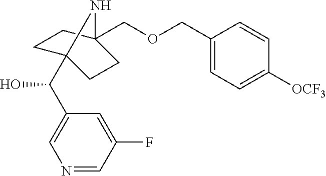 [SMILES: O[C@@H](c1cncc(F)c1)C12CCC(COCc3ccc(OC(F)(F)F)cc3)(CC1)N2] | (S)-(5-fluoro-3- pyridyl)(4-{[(p- trifluoromethoxyphenyl) methoxy]methyl}-7- azabicyclo[2.2.1]hept- 1-yl)methanol | − | − |
| 490 | 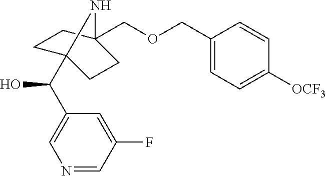 [SMILES: O[C@H](c1cncc(F)c1)C12CCC(COCc3ccc(OC(F)(F)F)cc3)(CC1)N2] | (R)-(5-fluoro-3- pyridyl)(4-{[(p- trifluoromethoxyphenyl) methoxy]methyl}-7- azabicyclo[2.2.1]hept- 1-yl)methanol | ++ | − |
| 491 | 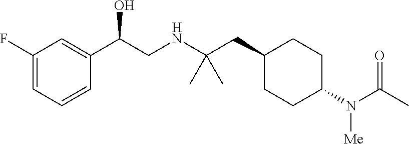 [SMILES: CC(=O)N(C)[C@H]1CC[C@H](CC(C)(C)NC[C@H](O)c2cccc(F)c2)CC1] | N-[(1R,4r)-4-{2-[(R)-2- (m-fluorophenyl)-2- hydroxyethylamino]-2- methylpropyl}cyclohexyl]- N-methylacetamide | +++ | − |
| 492 | 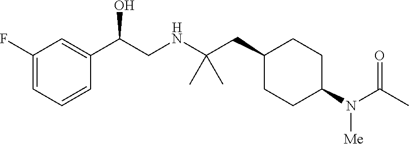 [SMILES: CC(=O)N(C)[C@H]1CC[C@@H](CC(C)(C)NC[C@H](O)c2cccc(F)c2)CC1] | N-[(1S,4s)-4-{2-[(R)-2- (m-fluorophenyl)-2- hydroxyethylamino]-2- methylpropyl}cyclohexyl]- N-methylacetamide | + | − |
| 493 | 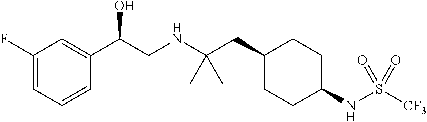 [SMILES: CC(C)(C[C@H]1CC[C@@H](NS(=O)(=O)C(F)(F)F)CC1)NC[C@H](O)c1cccc(F)c1] | (R)-2-{1,1-dimethyl-2- [(1s,4S)-4- (trifluoromesylamino)cy- clohexyl]ethylamino}-1- (m-fluorophenyl)-1- ethanol | + | − |
| 494 | 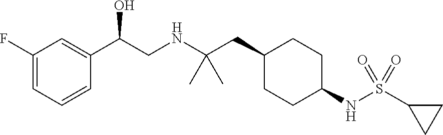 [SMILES: CC(C)(C[C@H]1CC[C@@H](NS(=O)(=O)C2CC2)CC1)NC[C@H](O)c1cccc(F)c1] | (R)-2-{1,1-dimethyl-2- [(1s,4S)-4- (cyclopropylsuflonylami- no)cyclohexyl]ethylamino}- 1-(m-fluorophenyl)- 1-ethanol | + | − |
| 495 | 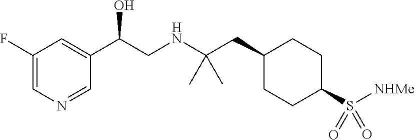 [SMILES: CNS(=O)(=O)[C@H]1CC[C@@H](CC(C)(C)NC[C@H](O)c2cncc(F)c2)CC1] | (R)-2-{1,1-dimethyl-2- [(1s,4S)-4- (methylaminosulfonyl)cy- clohexyl]ethylamino}- 1-(5-fluoro-3-pyridyl)- 1-ethanol | ++ | − |
| 496 | 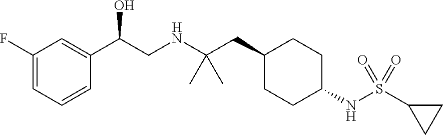 [SMILES: CC(C)(C[C@H]1CC[C@H](NS(=O)(=O)C2CC2)CC1)NC[C@H](O)c1cccc(F)c1] | (R)-2-{1,1-dimethyl-2- [(1r,4R)-4- (cyclopropylsulfonylami- no)cyclohexyl]ethylamino}- 1-(m-fluorophenyl)- 1-ethanol | ++ | − |
| 497 | 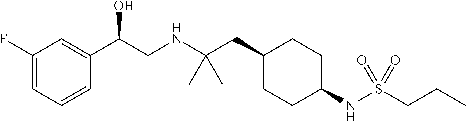 [SMILES: CCCS(=O)(=O)N[C@H]1CC[C@@H](CC(C)(C)NC[C@H](O)c2cccc(F)c2)CC1] | (R)-2-{1,1-dimethyl-2- [(1s,4S)-4- (propylsulfonylamino)cy- clohexyl]ethylamino}-1- (m-fluorophenyl)-1- ethanol | ++ | − |
| 498 | 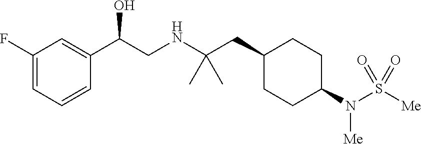 [SMILES: CN([C@H]1CC[C@@H](CC(C)(C)NC[C@H](O)c2cccc(F)c2)CC1)S(C)(=O)=O] | (R)-2-{1,1-dimethyl-2- [(1s,4S)-4-(N-mesyl-N- methylamino)cyclohexyl] ethylamino}-1-(m- fluorophenyl)-1-ethanol | + | − |
| 499 | 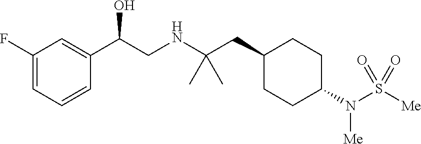 [SMILES: CN([C@H]1CC[C@H](CC(C)(C)NC[C@H](O)c2cccc(F)c2)CC1)S(C)(=O)=O] | (R)-2-{1,1-dimethyl-2- [(1r,4R)-4-(N-mesyl-N- methylamino)cyclohexyl] ethylamino}-1-(m- fluorophenyl)-1-ethanol | + | − |
| 500 | 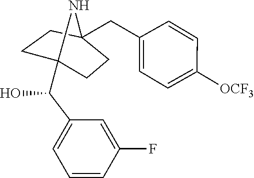 [SMILES: O[C@@H](c1cccc(F)c1)C12CCC(Cc3ccc(OC(F)(F)F)cc3)(CC1)N2] | (S)-(m-fluorophenyl({4- [(p- trifluoromethoxyphenyl) methyl]-7- azabicyclo[2.2.1]hept- 1-yl}methanol | − | − |
| 501 | 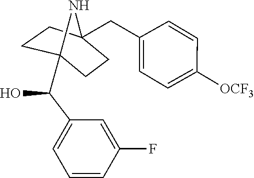 [SMILES: O[C@H](c1cccc(F)c1)C12CCC(Cc3ccc(OC(F)(F)F)cc3)(CC1)N2] | (R)-(m-fluorophenyl){4- [(p- trifluoromethoxyphenyl) methyl]-7- azabicyclo[2.2.1]hept- 1-yl}methanol | − | − |
| 502 | 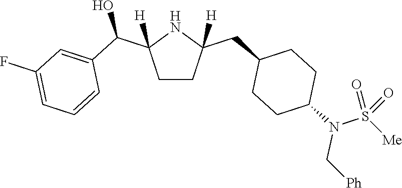 [SMILES: CS(=O)(=O)N(Cc1ccccc1)[C@H]1CC[C@H](C[C@@H]2CC[C@H]([C@H](O)c3cccc(F)c3)N2)CC1] | (R)-[(2R,5S)-5- ({(1r,4S)-4-[(benzyl)- N- mesylamino]cyclohexyl} methyl)-2- pyrrolidinyl](m- fluorophenyl)methanol | − | − |
| 503 | 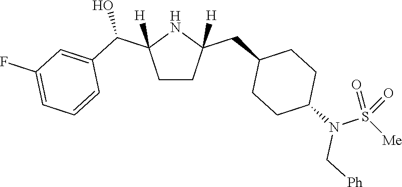 [SMILES: CS(=O)(=O)N(Cc1ccccc1)[C@H]1CC[C@H](C[C@@H]2CC[C@H]([C@@H](O)c3cccc(F)c3)N2)CC1] | (S)-[(2R,5S)-5- ({(1r,4S)-4-[(benzyl)- N- mesylamino]cyclohexyl} methyl)-2- pyrrolidinyl](m- fluorophenyl)methanol | − | − |
| 504 | 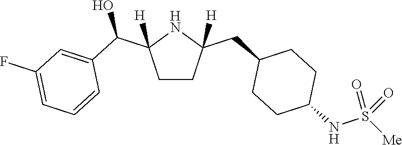 [SMILES: CS(=O)(=O)N[C@H]1CC[C@H](C[C@@H]2CC[C@H]([C@H](O)c3cccc(F)c3)N2)CC1] | (R)-[(2R,5S)-5- {[(1r,4S)-4- (mesylamino)cyclohexyl] methyl}-2- pyrrolidinyl](m- fluorophenyl)methanol | ++ | − |
| 505 | 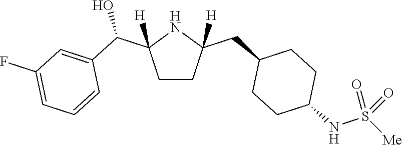 [SMILES: CS(=O)(=O)N[C@H]1CC[C@H](C[C@@H]2CC[C@H]([C@@H](O)c3cccc(F)c3)N2)CC1] | (S)-[(2R,5S)-5- {[(1r,4S)-4- (mesylamino)cyclohexyl] methyl}-2- pyrrolidinyl](m- fluorophenyl)methanol | − | − |
| 506 | 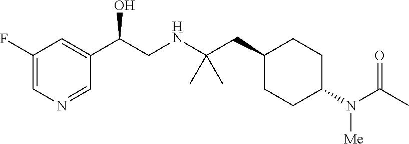 [SMILES: CC(=O)N(C)[C@H]1CC[C@H](CC(C)(C)NC[C@H](O)c2cncc(F)c2)CC1] | N-[(1R,4r)-4-{2-[(R)-2- (5-fluoro-3-pyridyl)-2- hydroxyethylamino]-2- methylpropyl}cyclohexyl]- N-methylacetamide | +++ | + |
| 507 | 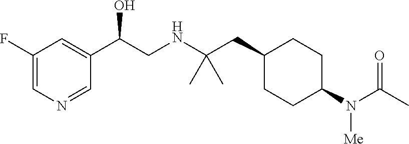 [SMILES: CC(=O)N(C)[C@H]1CC[C@@H](CC(C)(C)NC[C@H](O)c2cncc(F)c2)CC1] | N-[(1S,4s)-4-{2-[(R)-2- (5-fluoro-3-pyridyl)-2- hdyroxyethylamino]-2- methylpropyl}cyclohexyl]- n-methylacetamide | ++ | − |
| 508 | 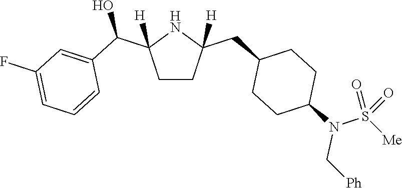 [SMILES: CS(=O)(=O)N(Cc1ccccc1)[C@H]1CC[C@@H](C[C@@H]2CC[C@H]([C@H](O)c3cccc(F)c3)N2)CC1] | (R)-[(2R,5S)-5- ({(1s,4R)-4-[(benzyl)- N- mesylamino]cyclohexyl} methyl)-2- pyrrolidinyl](m- fluorophenyl)methanol | − | − |
| 509 | 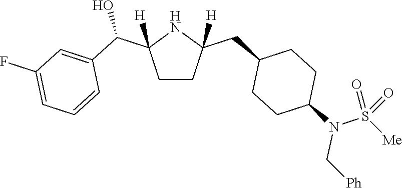 [SMILES: CS(=O)(=O)N(Cc1ccccc1)[C@H]1CC[C@@H](C[C@@H]2CC[C@H]([C@@H](O)c3cccc(F)c3)N2)CC1] | (S)-[(2R,5S)-5- ({(1s,4R)-4-[(benzyl)- N- mesylamino]cyclohexyl} methyl)-2- pyrrolidinyl](m- fluorophenyl)methanol | − | − |
| 510 | 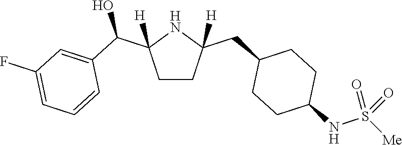 [SMILES: CS(=O)(=O)N[C@H]1CC[C@@H](C[C@@H]2CC[C@H]([C@H](O)c3cccc(F)c3)N2)CC1] | (R)-[(2R,5S)-5- {[(1s,4R)-4- (mesylamino)cyclohexyl] methyl}-2- pyrrolidinyl](m- fluorophenyl)methanol | +++ | − |
| 511 | 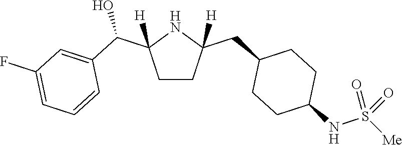 [SMILES: CS(=O)(=O)N[C@H]1CC[C@@H](C[C@@H]2CC[C@H]([C@@H](O)c3cccc(F)c3)N2)CC1] | (S)-[(2R,5S)-5- {[(1s,4R)-4- (mesylamino)cyclohexyl] methyl}-2- pyrrolidinyl](m- fluorophenyl)methanol | + | − |
| 512 | 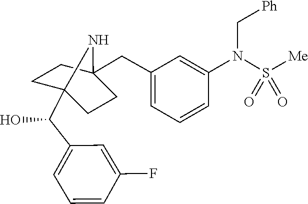 [SMILES: CS(=O)(=O)N(Cc1ccccc1)c1cccc(CC23CCC([C@@H](O)c4cccc(F)c4)(CC2)N3)c1] | (S)-[4-({m-[(benzyl)-N- mesylamino]phenyl}meth- yl)-7- azabicyclo[2.2.1]hept- 1-yl](m- fluorophenyl)methanol | − | − |
| 513 | 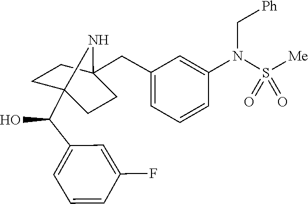 [SMILES: CS(=O)(=O)N(Cc1ccccc1)c1cccc(CC23CCC([C@H](O)c4cccc(F)c4)(CC2)N3)c1] | (R)-[4-({m-[(benzyl)-N- mesylamino]phenyl}meth- yl)-7- azabicyclo[2.2.1]hept- 1-yl](m- fluorophenyl)methanol | + | − |
| 514 | 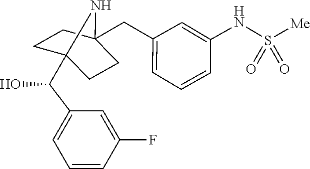 [SMILES: CS(=O)(=O)Nc1cccc(CC23CCC([C@@H](O)c4cccc(F)c4)(CC2)N3)c1] | (S)-(m-fluorophenyl)(4- {[m- (mesylamino)phenyl]meth- yl}-7- azabicyclo[2.2.1]hept- 1-yl)methanol | ++ | − |
| 515 | 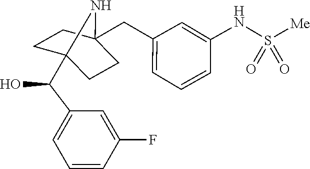 [SMILES: CS(=O)(=O)Nc1cccc(CC23CCC([C@H](O)c4cccc(F)c4)(CC2)N3)c1] | (R)-(m-fluorophenyl)(4- {[m- (mesylamino)phenyl]meth- yl}-7- azabicyclo[2.2.1]hept- 1-yl)methanol | ++ | + |
| 516 | 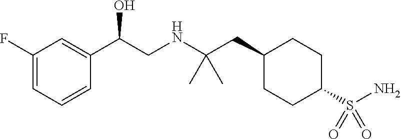 [SMILES: CC(C)(C[C@H]1CC[C@H](S(N)(=O)=O)CC1)NC[C@H](O)c1cccc(F)c1] | (1R,4r)-4-{2-[(R)-2- (m-fluorophenyl)-2- hydroxyethylamino]-2- methylpropyl}cyclohexane- sulfonamide | +++ | − |
| 517 | 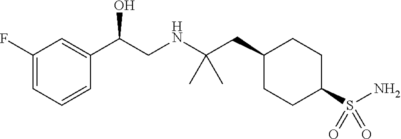 [SMILES: CC(C)(C[C@H]1CC[C@@H](S(N)(=O)=O)CC1)NC[C@H](O)c1cccc(F)c1] | (1S,4s)-4-{2-[(R)-2- (m-fluorophenyl)-2- hydroxyethylamino]-2- methylpropyl}cyclohexane- sulfonamide | + | − |
| 518 | 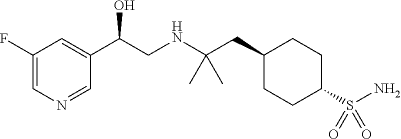 [SMILES: CC(C)(C[C@H]1CC[C@H](S(N)(=O)=O)CC1)NC[C@H](O)c1cncc(F)c1] | (1R,4r)-4-{2-[(R)-2-(5- fluoro-3-pyridyl)-2- hydroxyethylamino]-2- methylpropyl}cyclohexane- sulfonamide | ++ | − |
| 519 | 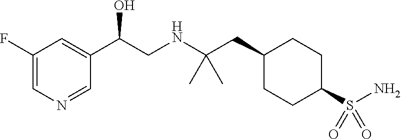 [SMILES: CC(C)(C[C@H]1CC[C@@H](S(N)(=O)=O)CC1)NC[C@H](O)c1cncc(F)c1] | (1S,4s)-4-{2-[(R)-2-(5- fluoro-3-pyridyl)-2- hydroxyethylamino]-2- methylpropyl}cyclohexane- sulfonamide | +++ | − |
| 520 | 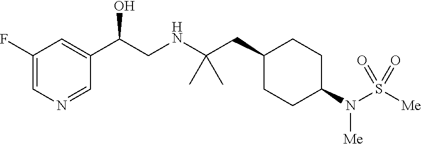 [SMILES: CN([C@H]1CC[C@@H](CC(C)(C)NC[C@H](O)c2cncc(F)c2)CC1)S(C)(=O)=O] | (R)-2-{1,1-dimethyl-2- [(1s,4S)-4-(N-mesyl-N- methylamino)cyclohexyl] ethylamino}-1-(5- fluoro-3-pyridyl)-1- ethanol | ++ | − |
| 521 | 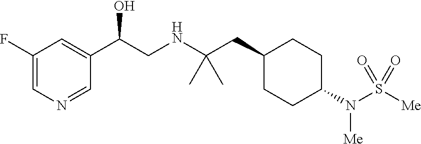 [SMILES: CN([C@H]1CC[C@H](CC(C)(C)NC[C@H](O)c2cncc(F)c2)CC1)S(C)(=O)=O] | (R)-2-{1,1-dimethyl-2- [(1r,4R)-4-(N-mesyl-N- methylamino)cyclohexyl] ethylamino}-1-(5- fluoro-3-pyridyl)-1- ethanol | ++ | − |
| 522 | 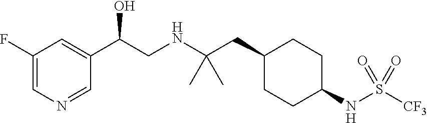 [SMILES: CC(C)(C[C@H]1CC[C@@H](NS(=O)(=O)C(F)(F)F)CC1)NC[C@H](O)c1cncc(F)c1] | (R)-2-{1,1-dimethyl-2- [(1s,4S)-4- (trifluoromesylamino)cy- chloehxyl]ethylamino}-1- (5-fluoro-3-pyridyl)-1- ethanol | +++ | − |
| 523 | 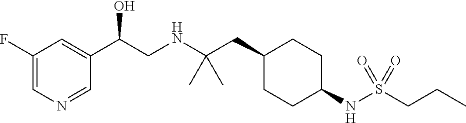 [SMILES: CCCS(=O)(=O)N[C@H]1CC[C@@H](CC(C)(C)NC[C@H](O)c2cncc(F)c2)CC1] | (R)-2-{1,1-dimethyl-2- [(1s,4S)-4- (propylsulfonylamin)cy- clohexyl]ethylamino}-1- (5-fluoro-3-pyridyl)-1- ethanol | ++ | − |
| 524 | 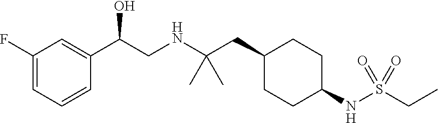 [SMILES: CCS(=O)(=O)N[C@H]1CC[C@@H](CC(C)(C)NC[C@H](O)c2cccc(F)c2)CC1] | (R)-2-{1,1-dimethyl-2- [(1s,4S)-4- (ethylsulfonylamino)cyclo- hexyl]ethylamino}-1- (m-fluorophenyl)-1- ethanol | ++ | − |
| 525 | 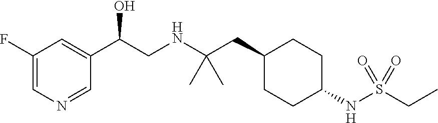 [SMILES: CCS(=O)(=O)N[C@H]1CC[C@H](CC(C)(C)NC[C@H](O)c2cncc(F)c2)CC1] | (R)-2-{1,1-dimethyl-2- [(1r,4R)-4- (ethylsulfonylamino)cyclo- hexyl]ethylamino}-1- (5-fluoro-3-pyridyl)-1- ethanol | ++ | − |
| 526 | 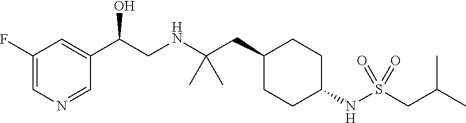 [SMILES: CC(C)CS(=O)(=O)N[C@H]1CC[C@H](CC(C)(C)NC[C@H](O)c2cncc(F)c2)CC1] | (R)-2-{1,1-dimethyl-2- [(1r,4R)-4- (isobutylsulfonylamino)cy- clohexyl]ethylamino}- 1-(5-fluoro-3-pyridyl)- 1-ethanol | ++ | − |
| 527 | 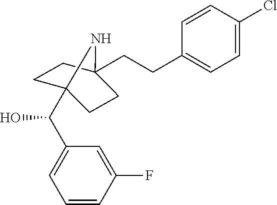 [SMILES: O[C@@H](c1cccc(F)c1)C12CCC(CCc3ccc(Cl)cc3)(CC1)N2] | (S)-{4-[2-(p- chlorophenyl)ethyl]-7- azabicyclo[2.2.1]hept- 1-yl}(m- fluorophenyl)methanol | − | − |
| 528 | 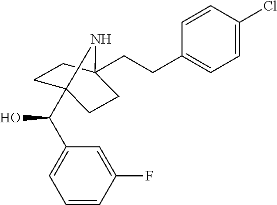 [SMILES: O[C@H](c1cccc(F)c1)C12CCC(CCc3ccc(Cl)cc3)(CC1)N2] | (R)-{4-[2-(p- chlorophenyl)ethyl]-7- azabicyclo[2.2.1]hept- 1-yl}(m- fluorophenyl)methanol | − | − |
| 529 | 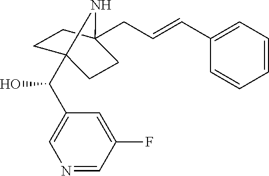 [SMILES: O[C@@H](c1cncc(F)c1)C12CCC(C/C=C/c3ccccc3)(CC1)N2] | (S)-{4-[(E)-3-phenyl-2- propenyl]-7- azabicyclo[2.2.1]hept- 1-yl}(5-fluoro-3- pyridyl)methanol | + | − |
| 530 | 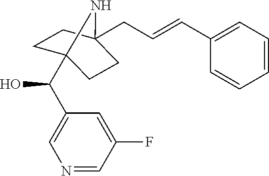 [SMILES: O[C@H](c1cncc(F)c1)C12CCC(C/C=C/c3ccccc3)(CC1)N2] | (R)-{4-[(E)-3-phenyl-2- propenyl]-7- azabicyclo8 2.2.1]hept- 1-yl}(5-fluoro-3- pyridyl)methanol | +++ | + |
| 531 | 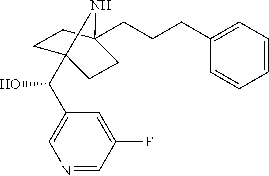 [SMILES: O[C@@H](c1cncc(F)c1)C12CCC(CCCc3ccccc3)(CC1)N2] | (S)-(5-fluoro-3- pyridyl){4-(3- phenylpropyl)-7- azabicyclo[2.2.1]hept- 1-yl}methanol | ++ | − |
| 532 | 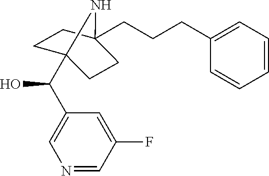 [SMILES: O[C@H](c1cncc(F)c1)C12CCC(CCCc3ccccc3)(CC1)N2] | (R)-(5-fluoro-3- pyridyl){4-(3- phenylpropyl)-7- azabicyclo[2.2.1]hept- 1-yl}methanol | ++ | + |
| 533 | 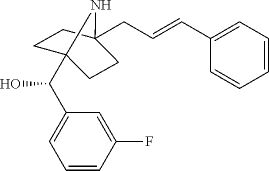 [SMILES: O[C@@H](c1cccc(F)c1)C12CCC(C/C=C/c3ccccc3)(CC1)N2] | (S)-{4-[(E)-3-phenyl-2- propenyl]-7- azabicyclo[2.2.1]hept- 1-yl}(m- fluorophenyl)methanol | − | − |
| 534 | 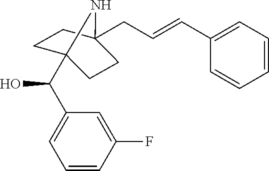 [SMILES: O[C@H](c1cccc(F)c1)C12CCC(C/C=C/c3ccccc3)(CC1)N2] | (R)-{4-[(E)-3-phenyl-2- propenyl]-7- azabicyclo[2.2.1]hept- 1-yl}(m- fluorophenyl)methanol | +++ | − |
| 535 | 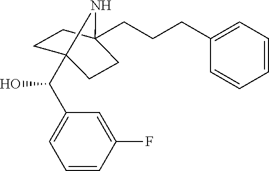 [SMILES: O[C@@H](c1cccc(F)c1)C12CCC(CCCc3ccccc3)(CC1)N2] | (S)-(m-fluorophenyl){4- (3-phenylpropyl)-7- azabicyclo[2.2.1]hept- 1-yl}methanol | + | − |
| 536 | 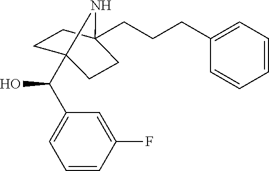 [SMILES: O[C@H](c1cccc(F)c1)C12CCC(CCCc3ccccc3)(CC1)N2] | (R)-(m-fluorophenyl){4- (3-phenylpropyl)-7- azabicyclo[2.2.1]hept- 1-yl}methanol | +++ | + |
| 537 | 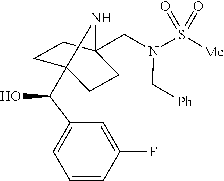 [SMILES: CS(=O)(=O)N(Cc1ccccc1)CC12CCC([C@H](O)c3cccc(F)c3)(CC1)N2] | (R)-(4-{[(benzyl)-N- mesylamino]methyl}-7- azabicyclo[2.2.1]hept- 1-yl)(m- fluorophenyl)methanol | ++ | + |
| 538 | 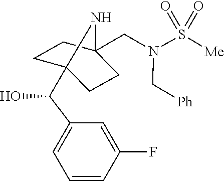 [SMILES: CS(=O)(=O)N(Cc1ccccc1)CC12CCC([C@@H](O)c3cccc(F)c3)(CC1)N2] | (S)-(4-{[(benzyl)-N- mesylamino]methyl}-7- azabicyclo[2.2.1]hept- 1-yl)(m- fluorophenyl)methanol | ++ | − |
| 539 | 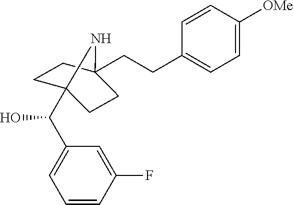 [SMILES: COc1ccc(CCC23CCC([C@@H](O)c4cccc(F)c4)(CC2)N3)cc1] | (S)-(m-fluorophenyl){4- [2-(p- methoxyphenyl)ethyl]- 7- azabicyclo[2.2.1]hept- 1-yl}methanol | − | − |
| 540 | 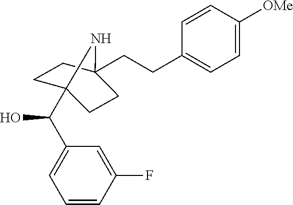 [SMILES: COc1ccc(CCC23CCC([C@H](O)c4cccc(F)c4)(CC2)N3)cc1] | (R)-(m-fluorophenyl){4- [2-(p- methoxyphenyl)ethyl]- 7- azabicyclo[2.2.1]hept- 1-yl}methanol | ++ | + |
| 541 | 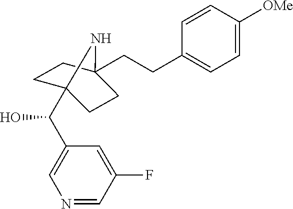 [SMILES: COc1ccc(CCC23CCC([C@@H](O)c4cncc(F)c4)(CC2)N3)cc1] | (S)-(5-fluoro-3- pyridyl){4-[2-(p- methoxyphenyl)ethyl]- 7- azabicyclo[2.2.1]hept- 1-yl}methanol | − | − |
| 542 | 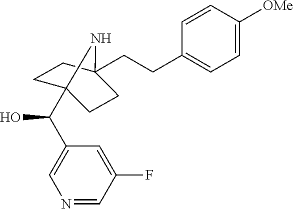 [SMILES: COc1ccc(CCC23CCC([C@H](O)c4cncc(F)c4)(CC2)N3)cc1] | (R)-(5-fluoro-3- pyridyl){4-[2-(p- methoxyphenyl)ethyl]- 7- azabicyclo[2.2.1]hept- 1-yl}methanol | ++ | + |
| 543 | 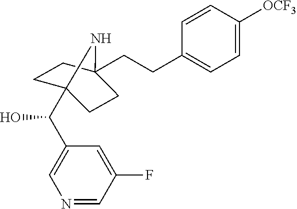 [SMILES: O[C@@H](c1cncc(F)c1)C12CCC(CCc3ccc(OC(F)(F)F)cc3)(CC1)N2] | (S)-(5-fluoro-3- pyridyl){4-[2-(p- trifluoromethoxyphenyl) ethyl]-7- azabicyclo[2.2.1]hept- 1-yl}methanol | − | − |
| 544 | 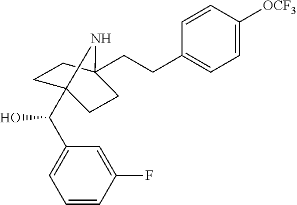 [SMILES: O[C@@H](c1cccc(F)c1)C12CCC(CCc3ccc(OC(F)(F)F)cc3)(CC1)N2] | (S)-(m-fluorophenyl){4- [2-(p- trifluoromethoxyphenyl) ethyl]-7- azabicyclo[2.2.1]hept- 1-yl}methanol | − | − |
| 545 | 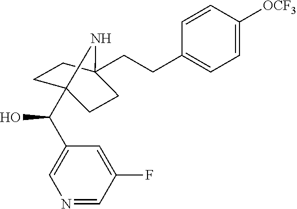 [SMILES: O[C@H](c1cncc(F)c1)C12CCC(CCc3ccc(OC(F)(F)F)cc3)(CC1)N2] | (R)-(5-fluoro-3- pryidyl){4-[2-(p- trifluoromethoxyphenyl) ethyl]-7- azabicyclo[2.2.1]hept- 1-yl}methanol | − | − |
| 546 | 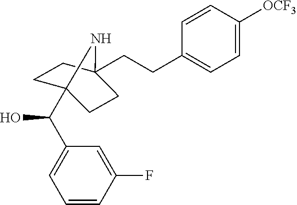 [SMILES: O[C@H](c1cccc(F)c1)C12CCC(CCc3ccc(OC(F)(F)F)cc3)(CC1)N2] | (R)-(m-fluorophenyl){4- [2-(p- trifluoromethoxyphenyl) ethyl]-7- azabicyclo[2.2.1]hept- 1-yl}methanol | − | − |
| 547 | 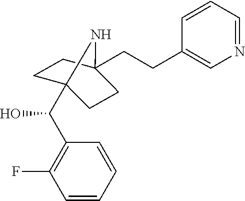 [SMILES: O[C@@H](c1ccccc1F)C12CCC(CCc3cccnc3)(CC1)N2] | (S)-(o-fluorophenyl){4- [2-(3-pyridyl)ethyl]-7- azabicyclo[2.2.1]hept- 1-yl}methanol | +++ | − |

In particular embodiments, the β₂-adrenergic receptor agonist is selected from the group consisting of:
2-(butylamino)-1-(p-hydroxyphenyl)-1-ethanol;
2-(tert-butylamino)-1-(p-hydroxyphenyl)-1-ethanol;
2-[(cyclohexylmethyl)amino]-1-(p-hydroxyphenyl)-1-ethanol;
(R)-2-(butylamino)-1-(p-hydroxyphenyl)-1-ethanol;
1-(m-hydroxyphenyl)-2-(1-methylbutylamino)-1-ethanol;
2-(3-cyclohexylpropylamino)-1-(p-hydroxyphenyl)-1-ethanol;
(R)-2-[(R)-1-methylbutylamino]-1-(p-hydroxyphenyl)-1-ethanol;
1-(4-amino-3,5-dichlorophenyl)-2-(butylamino)-1-ethanol;
2-(3-cyclopropylpropylamino)-1-(p-hydroxyphenyl)-1-ethanol;
1-[4-amino-3-chloro-5-(trifluoromethyl)phenyl]-2-(butylamino)-1-ethanol;
2-(butylamino)-1-(4-chloro-3-hydroxyphenyl)-1-ethanol;
1-(4-chloro-3-hydroxyphenyl)-2-(1-methylbutylamino)-1-ethanol;
m-{(R)-[(2R,5R)-5-methyl-2-pyrrolidinyl]hydroxymethyl}phenol and m-{(S)-[(2S,5S)-5-methyl-2-pyrrolidinyl]hydroxymethyl}phenol;
1-(3-amino-2,4-difluorophenyl)-2-(butylamino)-1-ethanol;
(R)-[(2R,6R)-6-propyl-2-piperidyl](m-hydroxyphenyl)methanol;
(R)-2-[(R)-1-methylbutylamino]-1-(m-fluorophenyl)-1-ethanol;
(R)-2-[(S)-1-methylbutylamino]-1-(m-fluorophenyl)-1-ethanol;
(R)-2-(tert-butylamino)-1-(m-fluorophenyl)-1-ethanol;
(R)-[(2R,5R)-5-propyl-2-pyrrolidinyl](m-fluorophenyl)methanol;
(R)-2-(1,1-dimethylbutylamino)-1-(m-fluorophenyl)-1-ethanol;
(R)-[(2R,6S)-6-propyl-2-piperidyl](m-hydroxyphenyl)methanol;
(R)-2-(tert-butylamino)-1-(2,3-difluorophenyl)-1-ethanol;
(R)-2-(butylamino)-1-(2,3-difluorophenyl)-1-ethanol;
(R)-2-(tert-butylamino)-1-(o-fluorophenyl)-1-ethanol;
(R)-2-(butylamino)-1-(o-fluorophenyl)-1-ethanol;
(R)-1-(m-fluorophenyl)-2-(1-methylcyclopropylamino)-1-ethanol;
(R)-1-(m-fluorophenyl)-2-(1-methylcyclobutylamino)-1-ethanol;
(R)-2-(butylamino)-1-(4-fluoro-3-hydroxyphenyl)-1-ethanol;
(R)-2-(butylamino)-1-(2-fluoro-3-hydroxyphenyl)-1-ethanol;
(R)-1-(3-amino-2-fluorophenyl)-2-(tert-butylamino)-1-ethanol;
4-[(R)-2-(tert-butylamino)-1-hydroxyethyl]-1-methyl-2(1H)-pyridinone;
(R)-2-(tert-butylamino)-1-(5-fluoro-3-pyridyl)-1-ethanol;
5-[(R)-2-(tert-butylamino)-1-hydroxyethyl]-3-pyridinol;
(R)-[(R)-5,5-dimethyl-2-pyrrolidinyl](m-fluorophenyl)methanol;
m-{(R)-[(R)-5,5-dimethyl-2-pyrrolidinyl]hydroxymethyl}phenol;
(R)-[(2S,6R)-6-propyl-2-piperidyl](m-chlorophenyl)methanol;
(S)-[(2S,6S)-6-propyl-2-piperidyl](m-hydroxyphenyl)methanol;
4-[2-(tert-butylamino)-1-hydroxyethyl]-3-pyridinol;
(R)-[(2S,6R)-6-propyl-2-piperidyl](m-hydroxyphenyl)methanol;
(R)-[(2R,5R)-5-propyl-2-pyrrolidinyl](o-chlorophenyl)methanol;
(R)-1-(3-amino-2,4-difluorophenyl)-2-(tert-butylamino)-1-ethanol;
(R)-2-(tert-butylamino)-1-(3-fluoro-2-tolyl)-1-ethanol;
(R)-[(R)-5,5-dimethyl-2-pyrrolidinyl](o-chlorophenyl)methanol;
(R)-[(R)-5,5-dimethyl-2-pyrrolidinyl](m-chlorophenyl)methanol;
(R)-[(R)-5,5-dimethyl-2-pyrrolidinyl](5-fluoro-3-pyridyl)methanol;
(R)-[(2R,5R)-5-propyl-2-pyrrolidinyl](5-fluoro-3-pyridyl)methanol;
(S)-[(R)-6,6-dimethyl-2-piperidyl](o-chlorophenyl)methanol;
(R)-[(R)-5,5-dimethyl-2-pyrrolidinyl](o-fluorophenyl)methanol;
(S)-[(R)-6,6-dimethyl-2-piperidyl](o-fluorophenyl)methanol;
(S)-(o-fluorophenyl)(4-methyl-7-azabicyclo[2.2.1]hept-1-yl)methanol;
(R)-(o-fluorophenyl)(4-methyl-7-azabicyclo[2.2.1]hept-1-yl)methanol;
(R)-(5-fluoro-3-pyridyl)(4-methyl-7-azabicyclo[2.2.1]hept-1-yl)methanol;
(R)-[(S)-6,6-dimethyl-2-piperidyl](m-fluorophenyl)methanol;
(R)-[(S)-6,6-dimethyl-2-piperidyl](o-fluorophenyl)methanol;
(R)-(o-chlorophenyl)(4-methyl-7-azabicyclo[2.2.1]hept-1-yl)methanol;
(R)-(m-chlorophenyl)(4-methyl-7-azabicyclo[2.2.1]hept-1-yl)methanol;
(R)-[(R)-4,4-dimethyl-2-azetidinyl](m-fluorophenyl)methanol;
(R)-[(R)-4,4-dimethyl-2-azetidinyl](o-fluorophenyl)methanol;
(R)-{(R)-1-methyl-2-azabicyclo[2.1.1]hex-3-yl}(m-chlorophenyl)methanol;
(S)-{(S)-1-methyl-2-azabicyclo[2.1.1]hex-3-yl}(o-fluorophenyl)methanol;
(S)-{(R)-1-methyl-2-azabicyclo[2.1.1]hex-3-yl}(o-fluorophenyl)methanol;
(R)-{(S)-1-methyl-2-azabicyclo[2.1.1]hex-3-yl}(o-fluorophenyl)methanol;
(R)-[(R)-4,4-dimethyl-2-azetidinyl](5-fluoro-3-pyridyl)methanol;
(R)-{(R)-1-methyl-2-azabicyclo[2.1.1]hex-3-yl}(5-fluoro-3-pyridyl)methanol;
(R)-[(R)-5,5-dimethyl-2-pyrrolidinyl](3-amino-2-fluorophenyl)methanol;
(R)-[(R)-4,4-dimethyl-2-azetidinyl](m-chlorophenyl)methanol;
(R)-[(2S,7S)-7-propyl-2-azepanyl](5-fluoro-3-pyridyl)methanol;
(S)-[(R)-5,5-dimethyl-2-pyrrolidinyl](4-amino-3,5-difluorophenyl)methanol;
(R)-1-(5-fluoro-3-pyridyl)-2-{2-[(1s,4S)-4-methoxycyclohexyl]ethylamino}-1-ethanol;
(R)-1-(m-fluorophenyl)-2-{1-methyl-1-[(1r,4R)-4-methoxycyclohexyl]ethylamino}-1-ethanol;
(R)-1-(5-fluoro-3-pyridyl)-2-{1-methyl-1-[(1r,4R)-4-methoxycyclohexyl]ethylamino}-1-ethanol;
(R)-{(R)-1-methyl-2-azabicyclo[2.1.1]hex-3-yl}(3-amino-2-fluorophenyl)methanol;
(R)-2-{1,1-dimethyl-2-[(1s,4S)-4-methoxycyclohexyl]ethylamino}-1-(m-fluorophenyl)-1-ethanol;
(R)-2-{1,1-dimethyl-2-[(1s,4S)-4-methoxycyclohexyl]ethylamino}-1-(5-fluoro-3-pyridyl)-1-ethanol;
(R)-2-{1,1-dimethyl-2-[(1r,4R)-4-methoxycyclohexyl]ethylamino}-1-(m-fluorophenyl)-1-ethanol;
(R)-2-{1-[(1S,3R)-3-methoxycyclohexyl]-1-methylethylamino}-1-(m-fluorophenyl)-1-ethanol;
(R)-2-{1-[(1R,3S)-3-methoxycyclohexyl]-1-methylethylamino}-1-(m-fluorophenyl)-1-ethanol;
(R)-1-(5-fluoro-3-pyridyl)-2-{3-[(1s,4R)-4-methoxycyclohexyl]propylamino}-1-ethanol;
(R)-1-(5-fluoro-3-pyridyl)-2-({[(1s,4S)-4-(benzyloxy)cyclohexyl]methyl}amino)-1-ethanol;
(R)-2-{1,1-dimethyl-3-[(1s,4R)-4-methoxycyclohexyl]propylamino}-1-(m-fluorophenyl)-1-ethanol;
(R)-2-{1,1-dimethyl-3-[(1s,4R)-4-methoxycyclohexyl]propylamino}-1-(5-fluoro-3-pyridyl)-1-ethanol;
(R)-1-(m-fluorophenyl)-2-{1-methyl-1-[(1s,4S)-4-(benzyloxy)cyclohexyl]ethylamino}-1-ethanol;
(R)-2-{1,1-dimethyl-3-[(1s,4R)-4-hydroxycyclohexyl]propylamino}-1-(m-fluorophenyl)-1-ethanol;
(R)-2-{1,1-dimethyl-3-[(1s,4R)-4-hydroxycyclohexyl]propylamino}-1-(5-fluoro-3-pyridyl)-1-ethanol;
(R)-2-{1,1-dimethyl-2-[(1r,4R)-4-hydroxycyclohexyl]ethylamino}-1-(m-fluorophenyl)-1-ethanol;
(R)-2-{1,1-dimethyl-2-[(1s,4S)-4-hydroxycyclohexyl]ethylamino}-1-(5-fluoro-3-pyridyl)-1-ethanol;
N-[(1R,4r)-4-{[(R)-2-(m-fluorophenyl)-2-hydroxyethylamino]methyl}cyclohexyl]benzamide;
(R)-2-{1,1-dimethyl-3-[(1r,4S)-4-hydroxycyclohexyl]propylamino}-1-(o-fluorophenyl)-1-ethanol;
(R)-2-{1,1-dimethyl-3-[(1s,4R)-4-hydroxycyclohexyl]propylamino}-1-(o-fluorophenyl)-1-ethanol;
methyl (1S,4s)-4-{2-[(R)-2-(5-fluoro-3-pyridyl)-2-hydroxyethylamino]-2-methylpropyl}cyclohexanecarboxylate;
(S)-[(2R,5S)-5-{[(1r,4S)-4-methoxycyclohexyl]methyl}-2-pyrrolidinyl](m-fluorophenyl)methanol;
(R)-[(2R,5S)-5-{[(1r,4S)-4-methoxycyclohexyl]methyl}-2-pyrrolidinyl](m-fluorophenyl)methanol;
methyl (1S,4s)-4-{2-[(R)-2-(m-fluorophenyl)-2-hydroxyethylamino]-2-methylpropyl}cyclohexanecarboxylate;
(1S,4s)-8-[(R)-2-(m-fluorophenyl)-2-hydroxyethylamino]-7-methoxy-p-menthan-7-one;
(R)-(m-fluorophenyl){4-(methoxymethyl)-7-azabicyclo[2.2.1]hept-1-yl}methanol;
(R)-[(2R,5R)-5-{[(1r,4R)-4-methoxycyclohexyl]methyl}-2-pyrrolidinyl](m-fluorophenyl)methanol;
(R)-[(2R,5S)-5-{[(1r,4S)-4-methoxycyclohexyl]methyl}-2-pyrrolidinyl](o-fluorophenyl)methanol;
(R)-[(2R,5R)-5-{[(1r,4R)-4-methoxycyclohexyl]methyl}-2-pyrrolidinyl](o-fluorophenyl)methanol;
(R)-[(2R,5R)-5-{[(1r,4R)-4-methoxycyclohexyl]methyl}-2-pyrrolidinyl](5-fluoro-3-pyridyl)methanol;
methyl (1R,4r)-4-{3-[(R)-2-(5-fluoro-3-pyridyl)-2-hydroxyethylamino]-3-methylbutyl}cyclohexanecarboxylate;
N-[(1R,4r)-4-{1-[(R)-2-(5-fluoro-3-pyridyl)-2-hydroxyethylamino]-1-methylethyl}cyclohexyl]acetamide;
(1S,4s)-4-{3-[(R)-2-(5-fluoro-3-pyridyl)-2-hydroxyethylamino]-3-methylbutyl}cyclohexanecarboxylic acid;
methyl (1S,4s)-4-{3-[(R)-2-(5-fluoro-3-pyridyl)-2-hydroxyethylamino]-3-methylbutyl}cyclohexanecarboxylate;
methyl (1R,4r)-4-{3-[(R)-2-(m-fluorophenyl)-2-hydroxyethylamino]-3-methylbutyl}cyclohexanecarboxylate;
methyl (1S,4s)-4-{3-[(R)-2-(m-fluorophenyl)-2-hydroxyethylamino]-3-methylbutyl}cyclohexanecarboxylate;
methyl (1R,4r)-4-{2-[(R)-2-(m-fluorophenyl)-2-hydroxyethylamino]-2-methylpropyl}cyclohexanecarboxylate;
N-[(1R,4r)-4-{2-[(R)-2-(5-fluoro-3-pyridyl)-2-hydroxyethylamino]-2-methylpropyl}cyclohexyl]acetamide;
(R)-2-{1,1-dimethyl-2-[(1r,4R)-4-aminocyclohexyl]ethylamino}-1-(5-fluoro-3-pyridyl)-1-ethanol;
(R)-2-{1,1-dimethyl-2-[(1r,4R)-4-(mesylamino)cyclohexyl]ethylamino}-1-(5-fluoro-3-pyridyl)-1-ethanol;
N-[(1R,4r)-4-{3-[(R)-2-(5-fluoro-3-pyridyl)-2-hydroxyethylamino]-3-methylbutyl}cyclohexyl]acetamide;
(S)-[(2R,5S)-5-methyl-5-{[(1r,4S)-4-methoxycyclohexyl]methyl}-2-pyrrolidinyl](m-fluorophenyl)methanol;
(S)-{(2R,5R)-5-[(p-methoxyphenyl)methyl]-5-methyl-2-pyrrolidinyl}(m-fluorophenyl)methanol;
(R)-{(2R,5R)-5-[(p-methoxyphenyl)methyl]-5-methyl-2-pyrrolidinyl}(m-fluorophenyl)methanol;
(1R,4r)-8-[(R)-2-(m-fluorophenyl)-2-hydroxyethylamino]-7-methoxy-p-menthan-7-one;
(1R,4r)-8-[(R)-2-(5-fluoro-3-pyridyl)-2-hydroxyethylamino]-7-methoxy-p-menthan-7-one;
(R)-2-{1,1-dimethyl-2-[(1r,4R)-4-aminocyclohexyl]ethylamino}-1-(m-fluorophenyl)-1-ethanol;
(S)-{(2R,5S)-5-[(p-methoxyphenyl)methyl]-2-pyrrolidinyl}(m-fluorophenyl)methanol;
(R)-{(2R,5S)-5-[(p-methoxyphenyl)methyl]-2-pyrrolidinyl}(m-fluorophenyl)methanol;
(R)-{(2R,5R)-5-[(p-methoxyphenyl)methyl]-2-pyrrolidinyl}(m-fluorophenyl)methanol;
(S)-{(2R,5R)-5-[(p-methoxyphenyl)methyl]-2-pyrrolidinyl}(m-fluorophenyl)methanol;
(R)-2-(tert-butylamino)-1-(2-methyl-3-pyridyl)-1-ethanol;
(S)-{(2R,5S)-5-[(p-methoxyphenyl)methyl]-2-pyrrolidinyl}(5-fluoro-3-pyridyl)methanol;
(R)-{(2R,5S)-5-[(p-methoxyphenyl)methyl]-2-pyrrolidinyl}(5-fluoro-3-pyridyl)methanol;
(R)-{(2R,5R)-5-[(p-methoxyphenyl)methyl]-2-pyrrolidinyl}(5-fluoro-3-pyridyl)methanol;
(S)-{(2R,5R)-5-[(p-methoxyphenyl)methyl]-2-pyrrolidinyl}(5-fluoro-3-pyridyl)methanol;
(R)-2-{1,1-dimethyl-2-[(1r,4R)-4-(mesylamino)cyclohexyl]ethylamino}-1-(m-fluorophenyl)-1-ethanol;
N-[(1R,4r)-4-{2-[(R)-2-(m-fluorophenyl)-2-hydroxyethylamino]-2-methylpropyl}cyclohexyl]acetamide;
N-[(1R,4r)-4-{1-[(R)-2-(m-fluorophenyl)-2-hydroxyethylamino]-1-methylethyl}cyclohexyl]acetamide;
6-[(S)-2-(tert-butylamino)-1-hydroxyethyl]-2-pyridinecarbonitrile;
(R)-(4-benzyl-7-azabicyclo[2.2.1]hept-1-yl)(m-fluorophenyl)methanol;
(R)-{(2R,5S)-5-[(p-chlorophenyl)methyl]-2-pyrrolidinyl}(m-fluorophenyl)methanol;
(R)-{(2R,5R)-5-[(p-chlorophenyl)methyl]-2-pyrrolidinyl}(m-fluorophenyl)methanol;
(R)-(4-{[(p-chlorophenyl)methoxy]methyl}-7-azabicyclo[2.2.1]hept-1-yl)(m-fluorophenyl)methanol;
(S)-2-(tert-butylamino)-1-[6-(trifluoromethyl)-2-pyridyl]-1-ethanol;
ethyl [(1R,4r)-4-{1-[(R)-2-(m-fluorophenyl)-2-hydroxyethylamino]-1-methylethyl}cyclohexyloxy]acetate;
(1S,4s)-4-{2-[(R)-2-(m-fluorophenyl)-2-hydroxyethylamino]-2-methylpropyl}cyclohexanecarbonitrile;
(R)-(m-fluorophenyl){4-[(p-methoxyphenyl)methyl]-7-azabicyclo[2.2.1]hept-1-yl}methanol;
(R)-{(2R,5S)-5-[(p-chlorophenyl)methyl]-2-pyrrolidinyl}(5-fluoro-3-pyridyl)methanol;
(S)-{(2R,5R)-5-[(p-chlorophenyl)methyl]-2-pyrrolidinyl}(5-fluoro-3-pyridyl)methanol;
(R)-{(2R,5R)-5-[(p-chlorophenyl)methyl]-2-pyrrolidinyl}(5-fluoro-3-pyridyl)methanol;
ethyl [(1R,4r)-4-{1-[(R)-2-(5-fluoro-3-pyridyl)-2-hydroxyethylamino]-1-methylethyl}cyclohexyloxy]acetate;
(R)-(m-fluorophenyl){4-[(p-fluorophenyl)methyl]-7-azabicyclo[2.2.1]hept-1-yl}methanol;
6-[(S)-2-{1,1-dimethyl-2-[(1r,4S)-4-aminocyclohexyl]ethylamino}-1-hydroxyethyl]-2-pyridinecarbonitrile;
N-[(1R,4s)-4-{2-[(S)-2-(6-cyano-2-pyridyl)-2-hydroxyethylamino]-2-methylpropyl}cyclohexyl]acetamide;
N-[(1S,4r)-4-{2-[(S)-2-(6-cyano-2-pyridyl)-2-hydroxyethylamino]-2-methylpropyl}cyclohexyl]acetamide;
6-[(S)-2-{1,1-dimethyl-2-[(1r,4S)-4-(mesylamino)cyclohexyl]ethylamino}-1-hydroxyethyl]-2-pyridinecarbonitrile;
N-[(1R,4r)-4-{2-[(R)-2-(m-fluorophenyl)-2-hydroxyethylamino]-2-methylpropyl}cyclohexyl]2,2-dimethylpropionamide;
N-[(1R,4r)-4-{2-[(R)-2-(m-fluorophenyl)-2-hydroxyethylamino]-2-methylpropyl}cyclohexyl]trifluoroacetamide;
N-[(1R,4r)-4-{2-[(R)-2-hydroxy-2-(2-methyl-3-pyridyl)ethylamino]-2-methylpropyl}cyclohexyl]acetamide;
(R)-(m-fluorophenyl)(4-{[(p-fluorophenyl)methoxy]methyl}-7-azabicyclo[2.2.1]hept-1-yl)methanol;
(S)-{4-[(p-chlorophenoxy)methyl]-7-azabicyclo[2.2.1]hept-1-yl}(m-fluorophenyl)methanol;
(R)-{4-[(p-chlorophenoxy)methyl]-7-azabicyclo[2.2.1]hept-1-yl}(m-fluorophenyl)methanol;
(R)-(4-{[(p-fluorophenyl)methoxy]methyl}-7-azabicyclo[2.2.1]hept-1-yl)(5-fluoro-3-pyridyl)methanol;
(S)-(m-fluorophenyl){4-[2-(3-pyridyl)ethyl]-7-azabicyclo[2.2.1]hept-1-yl}methanol;
N-[(1S,4s)-4-{2-[(R)-2-(m-fluorophenyl)-2-hydroxyethylamino]-2-methylpropyl}cyclohexyl]cyclopropanecarboxamide;
N-[(1S,4s)-4-{2-[(R)-2-(m-fluorophenyl)-2-hydroxyethylamino]-2-methylpropyl}cyclohexyl]acetamide;
(R)-2-{1,1-dimethyl-2-[(1s,4S)-4-(mesylamino)cyclohexyl]ethylamino}-1-(m-fluorophenyl)-1-ethanol;
N-[(1S,4s)-4-{2-[(R)-2-(m-fluorophenyl)-2-hydroxyethylamino]-2-methylpropyl}cyclohexyl]trifluoroacetamide;
(R)-2-{1,1-dimethyl-2-[(1s,4S)-4-aminocyclohexyl]ethylamino}-1-(m-fluorophenyl)-1-ethanol;
(R)-2-{1,1-dimethyl-2-[(1s,4S)-4-(3,3-dimethylureido)cyclohexyl]ethylamino}-1-(m-fluorophenyl)-1-ethanol;
(R)-2-{1,1-dimethyl-2-[(1r,4R)-4-(trifluoromesylamino)cyclohexyl]ethylamino}-1-(m-fluorophenyl)-1-ethanol;
(1S,4r)-4-(2-{(S)-2-hydroxy-2-[6-(trifluoromethyl)-2-pyridyl]ethylamino}-2-methylpropyl)cyclohexyl 2-methyl-2-propanecarbamate;
N-[(1S,4s)-4-{2-[(R)-2-(5-fluoro-3-pyridyl)-2-hydroxyethylamino]-2-methylpropyl}cyclohexyl]acetamide;
(R)-2-{1,1-dimethyl-2-[(1s,4S)-4-(mesylamino)cyclohexyl]ethylamino}-1-(5-fluoro-3-pyridyl)-1-ethanol;
(1S,4s)-4-{2-[(R)-2-(5-fluoro-3-pyridyl)-2-hydroxyethylamino]-2-methylpropyl}cyclohexyl 2-methyl-2-propanecarbamate;
(R)-2-{1,1-dimethyl-2-[(1s,4S)-4-(dimethylaminosulfonyl)cyclohexyl]ethylamino}-1-(m-fluorophenyl)-1-ethanol;
(R)-2-{1,1-dimethyl-2-[(1r,4R)-4-(methylaminosulfonyl)cyclohexyl]ethylamino}-1-(m-fluorophenyl)-1-ethanol;
N-[(1R,4r)-4-{2-[(R)-2-(m-fluorophenyl)-2-hydroxyethylamino]-2-methylpropyl}cyclohexyl]-N-methylacetamide;
N-[(1R,4r)-4-{2-[(R)-2-(5-fluoro-3-pyridyl)-2-hydroxyethylamino]-2-methylpropyl}cyclohexyl]-N-methylacetamide;
(R)-[(2R,5S)-5-{[(1s,4R)-4-(mesylamino)cyclohexyl]methyl}-2-pyrrolidinyl](m-fluorophenyl)methanol;
(1R,4r)-4-{2-[(R)-2-(m-fluorophenyl)-2-hydroxyethylamino]-2-methylpropyl}cyclohexanesulfonamide;
(1S,4s)-4-{2-[(R)-2-(5-fluoro-3-pyridyl)-2-hydroxyethylamino]-2-methylpropyl}cyclohexanesulfonamide;
(R)-2-{1,1-dimethyl-2-[(1s,4S)-4-(trifluoromesylamino)cyclohexyl]ethylamino}-1-(5-fluoro-3-pyridyl)-1-ethanol;
(R)-{4-[(E)-3-phenyl-2-propenyl]-7-azabicyclo[2.2.1]hept-1-yl}(5-fluoro-3-pyridyl)methanol;
(R)-{4-[(E)-3-phenyl-2-propenyl]-7-azabicyclo[2.2.1]hept-1-yl}(m-fluorophenyl)methanol;
(R)-(m-fluorophenyl){4-(3-phenylpropyl)-7-azabicyclo[2.2.1]hept-1-yl}methanol; and
(S)-(o-fluorophenyl){4-[2-(3-pyridyl)ethyl]-7-azabicyclo[2.2.1]hept-1-yl}methanol,
   and pharmaceutically acceptable salts thereof.

### Therapeutic Agents

As described herein, the present invention relates to uses and methods comprising treatment and/or prophylaxis with:
(a) insulin or a therapeutic insulin derivative; or
(b) a therapeutic agent stimulating the release of insulin.

The skilled person will be aware of various forms in which insulin may be used in treatment and administered to, or by, patients in need thereof.

In a particular embodiment, the present invention relates to uses and methods comprising treatment with insulin or a therapeutic insulin derivative.

As used herein, the term “therapeutic insulin derivative” will refer to derivates of insulin that deliver an equivalent or enhanced biological response (i.e. the biological response when administered to a patient is of the same kind as achieved by administration of insulin, wherein the effect is at a therapeutically equivalent or enhanced level, such as by providing beneficially altered pharmacokinetics when compared to insulin). Such derivatives may also include combinations of insulin derivatives.

Particular forms of insulin and therapeutic insulin derivatives that may be mentioned include the following:
Short-acting insulin, such as
Humulin R U-100,
Novolin R FlexPen,
Novolin R ReliOn, and
Novolin R FlexPen ReliOn;
Rapid-acting insulin, such as
inhaled insulin (e.g. Afrezza),
insulin aspart (e.g. Fiasp, Fiasp FlexTouch, Fiasp PenFill, NovoLog, NovoLog FlexPen, NovoLog FlexTouch, NovoLog PenFill, ReliOn NovoLog, ReliOn NovoLog FlexPen),
insulin glulisine (e.g. Apidra, Apidra SoloStar),
insulin lispro (e.g. Admelog, Admelog SoloStar, Humalog, Humalog KwikPen, Humalog Junior KwikPen), and
insulin lispro-aabc (e.g. Lyumjev, Lyumjev KwikPen);
Intermediate-acting insulin, such as
insulin isophane (e.g. Humulin N U-100, Humulin N KwikPen, Novolin N, Novolin N FlexPen, Novolin N ReliOn, Novolin N FlexPen ReliOn);
Long-acting insulin, such as
insulin degludec (e.g. Tresiba, Tresiba FlexTouch),
insulin detemir (e.g. Levemir),
insulin glargine (e.g. Basaglar KwikPen, Lantus, Lantus SoloStar, Toujeo SoloStar, Toujeo Max SoloStar),
insulin glargine-yfgn (e.g. Semglee-yfgn), and
concentrated regular insulin (e.g. Humulin R U-500, Humulin R U-500 KwikPen);
Combination (premixed) insulins, such as
insulin aspart protamine/insulin aspart 70/30 (e.g. NovoLog Mix 70/30, NovoLog Mix 70/30 FlexPen),
insulin isophane/regular insulin 70/30 (e.g. Humulin 70/30, Humulin 70/30 KwikPen, Novolin 70/30, Novolin 70/30 FlexPen, Novolin 70/30 FlexPen ReliOn),
insulin lispro protamine/insulin lispro 50/50 (e.g. Humalog Mix 50/50, Humalog Mix 50/50 KwikPen), and
insulin lispro protamine/insulin lispro 75/25 (e.g. Humalog Mix 75/25, Humalog Mix 75/25 KwikPen).

In a particular embodiment, the present invention relates to uses and methods comprising treatment with a therapeutic agent stimulating the release of insulin.

As used herein, the term “therapeutic agent stimulating the release of insulin” will refer to a therapeutic active ingredient (i.e. a pharmaceutical) that upon administration to a patient (i.e. in use) stimulates the relate of insulin by the pancreas (which effect may be provided together with other biological effects). Such therapeutic agents may also be referred to as insulin secretagogues.

Particular therapeutic agents that stimulate the release of insulin that may be mentioned include:
Sulphonylureas, such as tolbutamide, glimerpiride, glipizide and glyburide (including micronized glyburide);
Glinides, such as repaglinide and nateglinide;
DPP-4 inhibitors, such as alogliptin, sitagliptin, saxagliptin and linagliptin;
GLP-1 receptor agonists (including GLP-1 derivatives), such as exenatide, exenatide extended-release, tirzepatide, liraglutide, lixisenatide, semaglutide, albiglutide and dulaglutide.

The skilled person will understand that GLP-1 receptor agonists, i.e. agonists of the Glucagon-like peptide 1 receptor, as well as derivatives thereof, mimic the action of Glucagon-like peptide 1.

The GLP-1 receptor agonist may have affinity to the GIP receptor, i.e. the Gastric Inhibitory Polypeptide receptor, in addition to the GLP-1 receptor. Alternatively, the GLP-1 receptor agonist may be free or substantially free from affinity to the GIP receptor.

The skilled person will understand that GLP-1 receptor agonist may be a peptide. Further, it may be provided as a liquid or suspension, in injectable form such as in a prefilled pen for injection and/or as an extended release formulation. In an example, the GLP-1 receptor agonist may administrated subcutaneously. The GLP-1 receptor agonist may be administered once or several times daily or weekly.

The following are examples of GLP-1 receptor agonists.

Exenatide, which has the CAS number 141758-74-9, is a peptide sold under the trade names Byetta and Bydureon. It may be administered subcutaneously and/or in a dosage from 5 micrograms to 2 mg.

Tirzepatide, which has the CAS number 2023788-19-2, is a linear polypeptide of 39 amino acids that has been chemically modified by lipidation. It is sold under the trade name Mounjaro. It may be administered subcutaneously as an injectable solution. The dosage may be from 2.5 mg/0.5 mL to 15 mg/0.5 mL.

Liraglutide, which has the CAS number 204656-20-2, is a peptide sold under the brand names Saxenda, Victoza and Xultophy. It may be administered subcutaneously as an injectable solution. The dosage may be from 0.6 mg to 1.8 mg such as once daily for a week or more. For example, the dosage may be 0.6 mg, 1.2 mg or 1.8 such as once daily for a week or more.

Lixisenatide, which has the CAS number 320367-13-3, sold under the brand names Lyxumia and Adlyxin. It may be administrated in injectable form such as subcutaneously. For example, it may be administrated once daily. The dosage may be from 10 micrograms to 20 micrograms such as once weekly.

Semaglutide, which has the CAS number 910463-68-2, is a peptide sold under the brand names Ozempic, Rybelsus and Wegovy. It may be administrated in injectable form, such as subcutaneously (Ozempic and Wegovy), or orally (Rybelsus). The dosage may be from 0.25 mg to 2 mg, administered weekly.

Albiglutide, which has the CAS number 782500-75-8, is a peptide sold under the trade names Eperzan and Tanzeum. It may also be referred to as GSK-716155. It may be administrated in injectable form such as subcutaneously. The dosage may be from 30 mg to 50 mg such as once weekly.

Dulaglutide, which has the CAS number 923950-08-7, is a peptide sold under the brand name Trulicity among others. It may be administered in a pen for injection. For instance, it may be administered subcutaneously. The dosage of Dulaglutide may be 0.75 mg/0.5 mL, 1.5 mg/0.5 mL, 3 mg/0.5 mL or 4.5 mg/0.5 mL.

### Pharmaceutical Formulations

The skilled person will understand that both the β₂-adrenergic receptor agonist, or pharmaceutically acceptable salt thereof, and the insulin or a therapeutic insulin derivative, or therapeutic agent stimulating the release of insulin, may be administered in the form of a pharmaceutical formulation, which may further comprise one or more pharmaceutically acceptable excipient.

Suitable pharmaceutical formulations may be commercially available or otherwise are described in the literature, such as, Remington, The Science and Practice of Pharmacy, 19th ed., Mack Printing Company, Easton, Pennsylvania (1995), and Martindale—The Complete Drug Reference (35^{th} Edition), and the documents referred to therein, the relevant disclosures in all of which documents are hereby incorporated by reference in their entirety. Otherwise, the preparation of suitable formulations, and in particular combined preparations including both a β₂-adrenergic receptor agonist, or pharmaceutically acceptable salts thereof, and insulin or a therapeutic insulin derivative, or a therapeutic agent stimulating the release of insulin, may be achieved by the skilled person using routine techniques.

References to pharmaceutically acceptable excipient(s) may be understood to include pharmaceutically acceptable, diluents, carriers and/or adjuvants, as known to those skilled in the art.

Accordingly, in a second aspect of the invention, there is provided a pharmaceutical formulation comprising:
(I) a compound that is
   (a) insulin or a therapeutic insulin derivative; or
   (b) a therapeutic agent stimulating the release of insulin, and
(II) a β₂-adrenergic receptor agonist, or a pharmaceutically acceptable salt thereof,
and optionally one or more pharmaceutically acceptable excipient,
which formulations may be referred to as the “formulations of the invention”, or the like.

In a particular embodiment, the pharmaceutical formulation is for use in the treatment and/or prophylaxis of a disease or disorder.

In a third aspect of the invention, there is provided a pharmaceutical formulation comprising:
(I) a compound that is
   (a) insulin or a therapeutic insulin derivative; or
   (b) a therapeutic agent stimulating the release of insulin, and
(II) a β₂-adrenergic receptor agonist, or a pharmaceutically acceptable salt thereof,
and optionally one or more pharmaceutically acceptable excipient, for use in for use in the treatment and/or prophylaxis of hyperglycaemia or a disorder characterized by hyperglycaemia.

In an alternative third aspect of the invention, there is provided a method for the treatment and/or prophylaxis of hyperglycaemia or a disorder characterized by hyperglycaemia, comprising administering to a patient in need thereof a therapeutically effective amount of a pharmaceutical formulation comprising:
(I) a compound that is
   (a) insulin or a therapeutic insulin derivative; or
   (b) a therapeutic agent stimulating the release of insulin, and
(II) a β₂-adrenergic receptor agonist, or a pharmaceutically acceptable salt thereof,
and optionally one or more pharmaceutically acceptable excipient.

For the avoidance of doubt, the formulations of the second and third aspects of the invention may have any of the particular features described above for the first aspect of the invention, including all combinations thereof.

### Combinations and Kits-of-Parts

The skilled person will understand that the invention may also relate to combination products comprising a β₂-adrenergic receptor agonist, or a pharmaceutically acceptable salt thereof, and insulin or a therapeutic insulin derivative, or a therapeutic agent stimulating the release of insulin, and uses thereof.

The skilled person will understand that the combination of a β₂-adrenergic receptor agonist, or a pharmaceutically acceptable salt thereof, and insulin or a therapeutic insulin derivative, or a therapeutic agent stimulating the release of insulin, may also be provided in the form of a kit-of-parts comprising the same.

In a fourth aspect of the invention, there is provided a combination or kit-of-parts comprising components:
(A) a pharmaceutical formulation comprising a β₂-adrenergic receptor agonist, or a pharmaceutically acceptable salt thereof, optionally in admixture with one or more pharmaceutically acceptable excipient, and
(B) a pharmaceutical formulation comprising insulin or a therapeutic insulin derivative, or a therapeutic agent stimulating the release of insulin, optionally in admixture with one or more pharmaceutically acceptable excipient,
wherein components (A) and (B) are each provided in a form that is suitable for administration in conjunction with the other.

In a particular embodiment, the kit-of-parts is for use in the treatment and/or prophylaxis of hyperglycaemia or a disorder characterized by hyperglycaemia.

In a particular embodiment, the kit-of-parts of the fourth aspect of the invention further comprises instructions to use each component in conjunction with the other the treatment of hyperglycaemia or a disorder characterized by hyperglycaemia.

In an alternative fourth aspect of the invention, there is provided a kit-of-parts comprising:
(I) one of components (A) or (B) as defined hereinabove, and
(II) instructions to use that component in conjunction with the other of the two components in the treatment of hyperglycaemia or a disorder characterized by hyperglycaemia.

For the avoidance of doubt, kits-of-parts of the fourth aspect of the invention may have any of the particular features described above for the first, second or thirds aspects of the invention, including all combinations thereof.

In certain embodiments, the kits-of-parts described herein may comprise more than one formulation including an appropriate quantity/dose of a β₂-adrenergic receptor agonist, or pharmaceutically acceptable salt and/or pro drug thereof, and/or more than one formulation including an appropriate quantity/dose of insulin or a therapeutic insulin derivative, or a therapeutic agent stimulating the release of insulin, in order to provide for repeat dosing. If more than one formulation (comprising either active compound) is present, such formulations may be the same, or may be different in terms of the dose of either compound, chemical composition(s) and/or physical form(s).

With respect to the treatments, formulations and kits-of-parts of the invention as described herein, references to treatment with or administration of each component will refer to said component being administered in conjunction with the other.

### Methods of Administration

The skilled person will understand that the present invention (i.e. the pharmaceutical formulations, kits-of-parts, compounds for use, uses and methods of treatment as described herein, including all embodiments and preferred features thereof) relates to two components, namely a β₂-adrenergic receptor agonist, or a pharmaceutically acceptable salt thereof, and insulin or a therapeutic insulin derivative, or a therapeutic agent stimulating the release of insulin, being administered in conjunction with each other.

References to each component being administered in conjunction with the other will include the components being administered, sequentially, separately or simultaneously, as part of a medical intervention directed towards treatment of the relevant condition. In particular, such references may include the components being administered sufficiently close in time to enable a beneficial effect for the patient that is greater, over the course of the treatment of the relevant condition, than if administered in the absence of the other component, over the same course of treatment.

Determination of whether a combination provides a greater beneficial effect in respect of, and over the course of, treatment of a particular condition will depend upon the condition to be treated or prevented, but may be achieved routinely by the skilled person.

The skilled person will understand that references to the components (i.e. the β₂-adrenergic receptor agonist, or a pharmaceutically acceptable salt thereof, and insulin or a therapeutic insulin derivative, or a therapeutic agent stimulating the release of insulin) being administered sequentially (and, therefore, separately) will include that individual doses of each component are administered (i.e. taken by the patient, such as being taken orally) within 48 hours (e.g. within 24 hours, 12 hours, 6 hours, 3 hours, 2 hours, 1 hour, 45 minutes, 30 minutes, 20 minutes or 10 minutes) of each other.

Similarly, references to the components being administered simultaneously (which may be in a combined form or separately) will include the components being administered (i.e. taken by the patient, such as being taken orally) at substantially the same time.

In a particular embodiment, the components are administered (i.e. taken by the patient, such as being taken orally) sequentially (and, therefore, as separate doses).

For example, in a particular embodiment, the components are administered sequentially at least 2 hours apart (i.e. the interval between the administration of each component to the patient, e.g. orally, is at least 4 hours), such as between 2 hours and 48 hours apart, or between 2 hours and 24 hours apart, or between 2 hours and 12 hours apart.

For the avoidance of doubt, references to the components as the β₂-adrenergic receptor agonist, or a pharmaceutically acceptable salt thereof, and insulin or a therapeutic insulin derivative, or a therapeutic agent stimulating the release of insulin, will include references to the respective components (II) and (I) of the third aspect of the invention, and the respective components (A) and (B) of the fourth aspect of the invention.

The skilled person will understand that compounds and pharmaceutical formulations as defined herein will normally be administered orally, intravenously, subcutaneously, buccally, rectally, dermally, transdermally, nasally, tracheally, bronchially, sublingually, intranasally, topically, by any other parenteral route or via inhalation, in a pharmaceutically acceptable dosage form. Pharmaceutical formulations as described herein will include compositions in the form of tablets, capsules or elixirs for oral administration, suppositories for rectal administration, sterile solutions or suspensions for parenteral or intramuscular administration, and the like.

In particular embodiments, compounds and pharmaceutical formulations as described herein are administered orally. As such, in certain embodiments pharmaceutical formulations as described herein may be described as oral pharmaceutical formulations.

Thus, in particular embodiments, the pharmaceutical formulation(s) is/are provided in a pharmaceutically acceptable dosage form, including tablets or capsules, liquid forms to be taken orally or by injection, suppositories, creams, gels, foams, transdermal patches, plasters, inhalants (e.g. to be applied intranasally). For the avoidance of doubt, in such embodiments, compounds of the invention may be present as a solid (e.g. a solid dispersion), liquid (e.g. in solution) or in other forms, such as in the form of micelles.

In more particular embodiments, the pharmaceutical formulation(s) is/are provided in a pharmaceutically acceptable oral dosage form, including tablets or capsules, which forms may be prepared using techniques known to those skilled in the art.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that the compounds described herein, and formulations and kits-of parts comprising the same, may be administered (for example, as formulations as described hereinabove) at varying doses, with suitable doses being readily determined by one of skill in the art.

For example, suitable doses of a β₂-adrenergic receptor agonist, or a pharmaceutically acceptable salt thereof, may include those discussed in the above-mentioned publications, as incorporated herein by reference.

Similarly, suitable doses of insulin, therapeutic insulin derivatives, and therapeutic agents stimulating the release of insulin may include those described herein and those known to those skilled in the art (including those indicated in relevant drug formularies, such as the British National Formulary 85^{th} Edition, the contents of which are incorporated herein by reference).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; however, there can, of course, be individual instances where higher or lower dosage ranges are merited, and such are included within the scope of this invention.

As described herein above, the skilled person will understand that the treatments (and methods of prophylaxis) as described here may further comprise (i.e. be combined with) additional (i.e. other) treatment(s) for the same condition. In particular, treatments (and methods of prophylaxis) described herein may be combined with other means for the treatment of hyperglycaemia or a disorder characterized by hyperglycaemia.

Such agents will be readily identified by those skilled in the art and include, in particular, such therapeutic agents that are commercially available (e.g. agents that the subject of a marketing authorization in one or more territory, such as a European or US marketing authorization).

### Preparation of Formulations and Kits-of-Parts

Pharmaceutical formulations and kits-of-parts as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a β₂-adrenergic receptor agonist, or a pharmaceutically acceptable salt thereof, and insulin or a therapeutic insulin derivative, or a therapeutic agent stimulating the release of insulin, with one or more pharmaceutically-acceptable excipients (e.g. an adjuvant, diluent and/or carrier).

There is further provided a method of preparing a kit-of-parts as defined hereinbefore, which method comprises bringing component (A) into association with component (B), thus rendering the two components suitable for administration in conjunction with each other. As such, references to bringing into association will mean that the two components are rendered suitable for administration in conjunction with each other.

Thus, in relation to the process for the preparation of a kit-of-parts as hereinbefore defined, by bringing the two components “into association with” each other, it is contemplated that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

The skilled person will understand that pharmaceutical formulations, kits-of-parts, methods and uses described herein may have the advantage that, in the treatment of the conditions mentioned hereinbefore, they may be more convenient for the physician and/or patient than, be more efficacious than, be less toxic than, have a broader range of activity than, be more potent than, produce fewer side effects than, or may have other useful pharmacological properties over, similar methods (treatments) known in the prior art whether for use in the above-stated indications or otherwise. In particular, such pharmaceutical formulations, kits of parts, methods and uses may have the advantage that they are more efficacious and/or exhibit advantageous properties in vivo.

Without wishing to be bound by theory, it is believed that β₂-adrenergic receptor agonists may act as insulin sensitizing agents, thus increasing the effect of insulin therapy, whether through administration of insulin or stimulation of insulin production, in the treatment of hyperglycaemia or a disorder characterized by hyperglycaemia, such as type 1 and type 2 diabetes.

Moreover, certain of the compounds acting as β₂-adrenergic receptor agonists, such as those described herein, are able to activate the β₂-adrenergic receptor without (or with only a minimal effect in) inducing cAMP production. It is thought that this allows for therapeutic effects as described herein to be obtained with lower levels of side effects than would result from other treatments.

The combination of these therapeutic agents is therefore useful in the treatment hyperglycaemia or a disorder characterized by hyperglycaemia, and may have advantages over compounds used in such methods as described in the prior art, such as the ability to administer one or more of the administered compounds at lower doses, to achieve greater effects and/or to achieve lower levels of adverse events.

### BRIEF DESCRIPTION OF THE FIGURES

FIG. 1 shows that the glucose uptake promoted by Compound A is inhibited in a dose-dependent manner by the selective β₂-adrenergic receptor antagonist ICI-118551.

FIG. 2 shows the fasting blood glucose level for a control, liraglutide, Compound A and a combination of liraglutide and Compound A as described in Biological Example 2.

FIG. 3 shows the fasting insulin level for a control, liraglutide, Compound A and a combination of liraglutide and Compound A as described in Biological Example 2.

FIG. 4 shows the HOMA-IR level for a control, liraglutide, Compound A and a combination of liraglutide and Compound A as described in Biological Example 2.

FIG. 5 shows the glucose tolerance for a control, liraglutide, Compound A and a combination of liraglutide and Compound A as described in Biological Example 2.

### EXAMPLES

The present invention is illustrated by way of the following examples, which are not intended to be limiting on the overall scope of the invention.

For the avoidance of doubt, in the case of a discrepancy between the name of the compound and the structure drawn in this specification, the structure should prevail.

Mice were purchased from Scanbur (Charles River).

### Example Compounds

The following compound, which be referred to herein as Compound A, was used in the biological examples provided herein in the salt form as specified (and, in the context of which, references to Compound A will refer to that salt form).

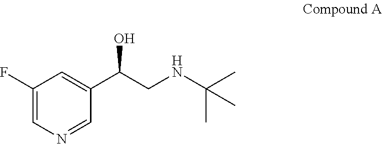

The synthesis of Compound A is described in WO 2019/053427 (see Example 17 therein), the contents of which are incorporated herein by reference.

### Compound Example 1

### Compound A: (R)-2-(tert-Butylamino)-1-(5-fluoropyridin-3-yl)ethan-1-ol

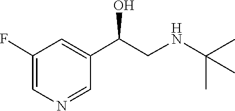

### (a) 2-Chloro-1-(5-fluoropyridin-3-yl)ethan-1-one

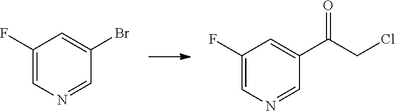 [SMILES: Fc1cncc(Br)c1 ; O=C(CCl)c1cncc(F)c1]

Isopropylmagnesium chloride (2 M in THF, 10.47 mL, 20.94 mmol) was added to a solution of LiCl (887.69 mg, 20.94 mmol) in THF (8 mL) at rt. After 15 min at rt, 3-bromo-5-fluoropyridine (3.35 g, 19.04 mmol) in THF (30 mL) was added dropwise at 0° C. The mixture was stirred at rt for 2 h and cooled in an ice-bath. A solution of 2-chloro-N-methoxy-N-methylacetamide (2.62 g, 19.04 mmol) in THF (30 mL) was added dropwise, and the mixture was stirred at rt for 2 h. NH₄Cl (aq, 10%) was added and the mixture was extracted with Et₂O. The combined extracts were washed with brine, dried (Na₂SO₄) and concentrated. The residue was purified by chromatography to give the sub-title compound (1.52 g, 20.94 mmol, 46%).

### (b) (R)-2-Chloro-1-(5-fluoropyridin-3-yl)ethan-1-one

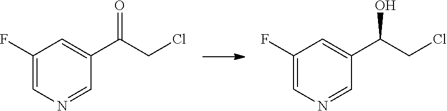 [SMILES: O=C(CCl)c1cncc(F)c1 ; O[C@@H](CCl)c1cncc(F)c1]

RhClCp*[(1S,2S)-p-TsNCH(C₆H₅)CH(C₆H₅)NH₂]/HCl·Et₃N (68.0 mg, 87.6 μmol), prepared from dichloro(pentamethylcyclopentadienyl)rhodium(III) dimer, (1S,2S)-(+)-N-(4-toluenesulphonyl)-1,2-diphenylethylene diamine and Et₃N as described in WO 2008/054155, was added to a mixture of 2-chloro-1-(5-fluoropyridin-3-yl)ethan-1-one (1.52 g, 8.76 mmol) in DMF (75 mL). Formic acid/Et₃N (5:2, 25 mL) was added and the mixture was stirred at rt for 15 min. H₂O (60 mL) and EtOAc (60 ml) where added and the layers were separated. The aq layer was washed with EtOAc (2×60 mL) and the combined organic phases were washed with H₂O, dried (Na₂SO₄), filtered and concentrated. The residue was purified by chromatography to give the sub-title compound (1.35 g, 7.69 mmol, 88%, ee=92.5%).

### (c) (R)-2-(tert-Butylamino)-1-(5-fluoropyridin-3-yl)ethan-1-ol

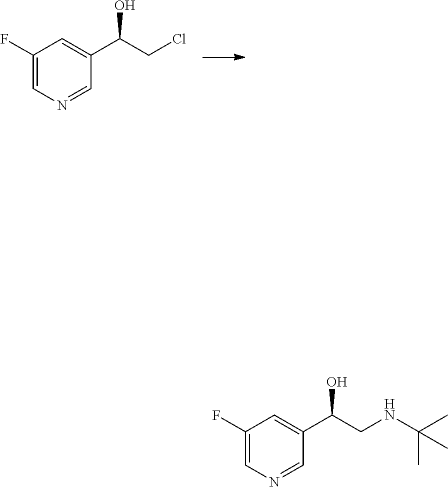

tert-Butylamine (11.37 mL, 108.21 mmol) followed by NaOH (476.08 mg, 11.90 mmol) were added to a mixture of 2-chloro-1-(5-fluoropyridin-3-yl)ethan-1-one (1.90 g, 10.82 mmol) and iPrOH (1.66 mL, 21.64 mmol) at rt. The mixture was heated at 75° C. for 4 h, allowed to cool, diluted with EtOAc, washed with H₂O and brine, dried (Na₂SO₄) and concentrated. The residue was dissolved in hot EtOAc and allowed to cool. Pentane was added and the mixture kept at −20° C. overnight. The solids were collected and purified by chromatography to give the title compound (1.43 g, 6.74 mmol, 62%, ee=98%).

¹H NMR (400 MHz, CDCl₃): δ 8.43-8.27 (m, 2H), 7.57-7.42 (m, 1H), 4.62 (dd, J=8.8, 3.7 Hz, 1H), 2.94 (dd, J=12.1, 3.8 Hz, 1H), 2.53 (dd, J=12.1, 8.8 Hz, 1H), 1.10 (s, 9H).

### Compound Example 2

### Compound A hemi-tartrate: (R)-2-(tert-Butylamino)-1-(5-fluoropyridin-3-yl)ethan-1-ol hemi-tartrate

A solution of L-(+)-tartaric acid (6.21 g, 0.5 eq) in EtOH (175 mL) was added to a solution of (R)-2-(tert-butylamino)-1-(5-fluoropyridin-3-yl)ethan-1-ol (17.57 g) in EtOH (525 mL, 30 vol) and H₂O (7 mL) at rt. The mixture was refluxed until all precipitate was dissolved, then cooled. The resultant slurry was stirred at rt overnight and then at 0 to 5° C. for 2 h. The solids were collected to give the title salt of (R)-2-(tert-butylamino)-1-(5-fluoropyridin-3-yl)ethan-1-ol (19.9 g, 83%, 100.0% purity by HPLC, 99.8% e.e. by HPLC).

Formation of the hemi-tartrate was confirmed by ¹H-NMR spectrum, which indicated an amine:acid ratio of 2:1.

### Compound Example 3

### Compound A dihydrochloride: (R)-2-(tert-Butylamino)-1-(5-fluoropyridin-3-yl)ethan-1-ol dihydrochloride

HCl (48 mmol, 2 M in Et₂O, 24 mL) was added dropwise to a solution of (R)-2-(tert-butylamino)-1-(5-fluoropyridin-3-yl)ethan-1-ol (5.10 g, 24 mmol) in Et₂O (90 mL) at rt. After 15 min the precipitate was collected, dried in vacuo and recrystallized from MeCN (250 mL). After having reached rt, the mixture was kept in the refrigerator for 5 h, and the solid was collected and dried in vacuo over P₂O₅ over 5 d to give the title compound, (6.41 g, 94%).

Elemental analysis found: C, 46.45%; H, 6.75%; N, 9.80%.

C₁₁H₁₉Cl₂FN₂ requires: C, 46.32%; H, 6.71%; N, 9.82%.

### Biological Examples

WO 2019/053427, the contents of which are incorporated herein by reference, describes biological assays showing that Compound A provides significant glucose uptake without inducing significant levels of cAMP (see Biological Examples 1 and 2 therein).

In the present examples: in Biological Example 1, Compound A was used in the form of the dihydrochloride (2HCl) salt; in Biological Example 2, Compound A was used in the form of the hemi-tartrate salt.

### Biological Example 1: Glucose Uptake in the Presence of a Selective β₂-Adrenergic Receptor Inhibitor

L6-myoblasts were grown in Dulbecco's Modified Eagle's Medium (DMEM) containing 1 g/L glucose supplemented with 10% fetal bovine serum (FBS), 2 mM L-glutamine, 50 U/mL penicillin, 50 g/mL streptomycin and 10 mM HEPES. Cells were plated at 1x 105 cells per mL in 24-well plates. After reaching 90% confluence the cells were grown in medium containing 2% FBS for 7 days where upon cells differentiated into myotubes. The differentiated L6-myotubes were serum-starved overnight in medium containing 0.5% fatty-acid free BSA and stimulated with Compound A at a final concentration of 1×10⁻⁵ M in the presence of the selective β₂-adrenergic receptor antagonist ICI-118551. After 1 h 40 min the cells were washed with warm, glucose free medium twice and another portion of agonist was added to the glucose free medium. After another 20 min of incubation the cells were exposed to 50 nM ³H-2-deoxyglucose for 10 min before washed in ice cold glucose free medium three times and lysed in 400 pl/well 0.2 M NaOH for 1 h at 60° C. The cell lysate was mixed with 4 ml scintillation buffer (Emulsifier Safe, Perkin Elmer) and the radioactivity was detected in a P-counter (Tri-Carb 4810TR, Perkin Elmer).

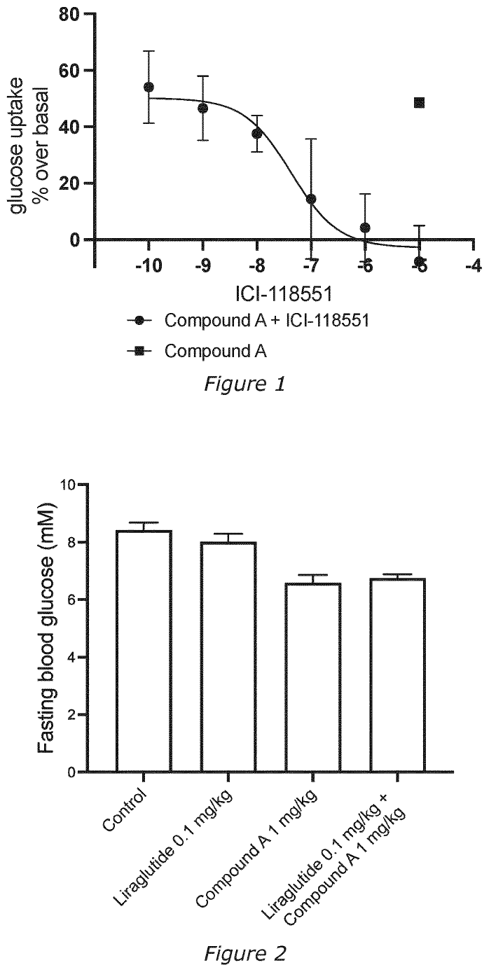

FIG. 1 shows that the glucose uptake promoted by Compound A is inhibited in a dose-dependent manner by the selective β₂-adrenergic receptor antagonist ICI-118551, which proves that the glucose uptake promoted by Compound A is mediated through the β₂-adrenergic receptor. In this document, the antagonist ICI-118551 is understood to have CAS number 72795-01-8.

### Biological Example 2: Combination of Compound A and Liraglutide

2.5 Months old male C57Bl/6N mice were grouped caged (4-5 mice per cage), kept at 30° C. and fed with high-fat, high-sucrose diet (45% fat) for 5.5 months. The mice were divided into four groups (2-3 mice per cage) according to their fasting blood glucose levels, glucose tolerance, body weight, body fat and lean mass, and treated daily for 2 weeks, subcutaneously with either vehicle, liraglutide (0.1 mg/kg), Compound A (1 mg/kg), or a mixture of liraglutide (0.1 mg/kg) and Compound A (1 mg/kg). The mice were fasted for 5 h and fasting blood glucose and fasting serum insulin levels were recorded. Glucose (2.5 g/kg lean mass) was injected ip and glucose levels were recorded after 15, 30, 60, 90 and 120 min. The results were used to calculate HOMA-IR (fasting blood glucose (mM)×fasting insulin (μIU/mL)/22.5) and glucose tolerance (area under the curves 0-120 min over the 5.5 mM blood glucose level).

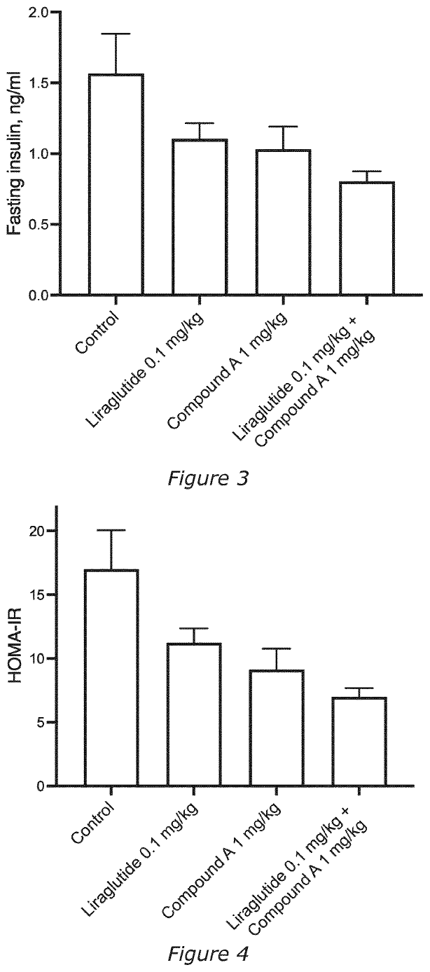

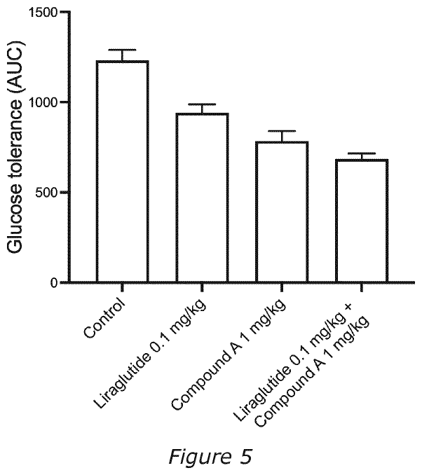

The results obtained over the period of the experiment are presented as follows: FIG. 2 shows fasting blood glucose levels; FIG. 3 shows fasting insulin levels; FIG. 4 shows HOMA-IR; FIG. 5 shows the result from the glucose tolerance test (measured as area under the curve over the 5.5 mM glucose level).

Compound A had additional positive effects on glucose homeostasis (glycemia, glucose tolerance and insulin sensitivity) when added in combination with liraglutide, which illustrates that combination treatment of Compound A with GLP1-RA or insulin can be beneficial for diabetic patients.

1. A β₂-adrenergic receptor agonist, or a pharmaceutically acceptable salt thereof, for use in the treatment and/or prophylaxis of hyperglycaemia or a disorder characterized by hyperglycaemia, wherein the treatment and/or prophylaxis further comprises administration of:
(a) insulin or a therapeutic insulin derivative; or
(b) a therapeutic agent stimulating the release of insulin.

2. A compound that is:
(a) insulin or a therapeutic insulin derivative; or
(b) a therapeutic agent stimulating the release of insulin
for use in the treatment and/or prophylaxis of hyperglycaemia or a disorder characterized by hyperglycaemia, wherein the use further comprises administration of a β₂-adrenergic receptor agonist, or a pharmaceutically acceptable salt thereof.

3. A combination of a β₂-adrenergic receptor agonist, or a pharmaceutically acceptable salt thereof, and a compound that is:
(a) insulin or a therapeutic insulin derivative; or
(b) a therapeutic agent stimulating the release of insulin
for use in the treatment and/or prophylaxis of hyperglycaemia or a disorder characterized by hyperglycaemia.

4. A method for the treatment and/or prophylaxis of hyperglycaemia or a disorder characterized by hyperglycaemia comprising:
(i) administration of a therapeutically effective amount of
(a) insulin or a therapeutic insulin derivative; or
(b) a therapeutic agent stimulating the release of insulin, and
(ii) administration of a therapeutically effective amount of a β₂-adrenergic receptor agonist, or a pharmaceutically acceptable salt thereof,
to a patient in need thereof.

5. A pharmaceutical formulation comprising:
(I) a compound that is
(a) insulin or a therapeutic insulin derivative; or
(b) a therapeutic agent stimulating the release of insulin, and
(II) a β₂-adrenergic receptor agonist, or a pharmaceutically acceptable salt thereof,
and optionally one or more pharmaceutically acceptable excipient.

6. A combination or kit-of-parts comprising components:
(A) a pharmaceutical formulation comprising a β₂-adrenergic receptor agonist, or a pharmaceutically acceptable salt thereof, optionally in admixture with one or more pharmaceutically acceptable excipient, and
(B) a pharmaceutical formulation comprising insulin or a therapeutic insulin derivative, or a therapeutic agent stimulating the release of insulin, optionally in admixture with one or more pharmaceutically acceptable excipient,
wherein components (A) and (B) are each provided in a form that is suitable for administration in conjunction with the other.

7. The formulation, combination or kit-of-parts of any one of claims 5 and 6 for use in the treatment and/or prophylaxis of hyperglycaemia or a disorder characterized by hyperglycaemia.

8. The β₂-adrenergic receptor agonist for use, compound for use, method, formulation for use, combination for use or kit-of-parts for use according to any one of claims 1 to 4 and 7, wherein the disorder characterised by hyperglycaemia is selected from the group consisting of Type 1 diabetes, Type 2 diabetes, Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

9. The β₂-adrenergic receptor agonist for use, compound for use, method, formulation, combination for use or kit-of-parts for use according to any one of claims 1 to 4 and 7 and 8, wherein the disorder characterised by hyperglycaemia is, or is characterised by, the patient displaying severe insulin resistance.

10. The β₂-adrenergic receptor agonist for use, compound for use, method, formulation, combination for use or kit-of-parts for use according to any one of claims 8 and 9, wherein the disorder characterised by hyperglycaemia is Type 1 diabetes or Type 2 diabetes.

11. The β₂-adrenergic receptor agonist for use, compound for use, method, formulation, combination, kit-of-parts, formulation for use, combination for use or kit of parts for use according to any one of claims 1 to 10, wherein the β₂-adrenergic receptor agonist is selected from the compounds of Table 1.

12. The β₂-adrenergic receptor agonist for use, compound for use, method, formulation, combination, kit-of-parts, formulation for use, combination for use or kit of parts for use according to any one of claims 1 to 11, wherein the β₂-adrenergic receptor agonist is: 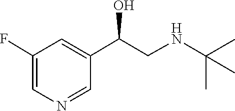
or a pharmaceutically acceptable salt thereof.

13. The β₂-adrenergic receptor agonist for use, compound for use, method, formulation, combination, kit-of-parts according, formulation for use, combination for use or kit of parts for use according to any one of claims 1 to 12, wherein the therapeutic agent stimulating the release of insulin is a GLP-1 receptor agonist.

14. The β₂-adrenergic receptor agonist for use, the compound for use, method, formulation, combination, kit-of-parts, formulation for use, combination for use or kit of parts for use according to claim 13, wherein the GLP-1 receptor agonist is liraglutide.
